(19) United States
(12) Patent Application Publication
(10) Pub. No.: US 2019/0247650 A1
(43) Pub. Date: Aug. 15, 2019

(54) SYSTEMS AND METHODS FOR AUGMENTING HUMAN MUSCLE CONTROLS

(71) Applicant: Bao Tran, (US)

(72) Inventor: Ha Tran, Saratoga, CA (US)

(21) Appl. No.: 15/896,063

(22) Filed: Feb. 14, 2018

(51) Int. Cl. A61N 1/36; A61N 1/02; A61N 1/362; A61N 1/37; G16H 50/20

(52) U.S. Cl. CPC A61N 1/3603; A61N 1/025; A61N 1/36003; A61B 5/021; A61N 1/3625; A61N 1/3704; G16H 50/20; A61N 1/36007

(57) ABSTRACT

Systems and methods are disclosed for physical assistance by: during a training phase, capturing muscle signals associated with a predetermined task and training a learning machine to associate the muscle signals with the task; during use, identifying a desired task to the learning machine to retrieve the muscle signals associated with the task; and applying functional electrical stimulation (FES) to actuate the muscle signals for the desired task.

### BACKGROUND

Millions of people are living with muscle control problems such as incontinence and spinal cord injury (SCI). To illustrate, SCI can be caused by diseases that destroy the neurological tissues of the spinal cord or by trauma that compresses, stretches, or severs this tissue. SCI is often irreversible, and can result in partial or total loss of sensory or motor function, or both, to the parts of the body below the level of the injury. For example, an injury to the spinal cord at the lower back usually affects the legs, but not the arms.

The most commonly used technology for restoring or replacing motor function in individuals with SCI is functional electrical stimulation (FES), which uses short electrical pulses to generate contractions in paralyzed muscles. These contractions can be coordinated to move or stabilize joints by stimulating one or more muscles that exert torques about the joint. The resulting joint angle or joint torque can be controlled by modulating the intensity of stimulation delivered to the flexor and extensor muscles, which actuate the joint in opposite directions.

### SUMMARY

Systems and methods are disclosed to capture muscle activities and model the activities as communications from the nervous system to the muscles. The decoded communications are then used to assist the user in a variety of applications, including medical, VR/AR, exoskeleton, walking, among others.

Advantages of the system may include one or more of the following. Walking in man is achieved by coordinated movements of all body segments using FES and artificial actuators. The mechanical actions, to which the metabolic energy consumption is associated, are effectively used through a skillful exploitation of the external and inertial forces. The natural and artificial motor strategies optimize are controlled by a hierarchical hybrid controller using FES and exoskeleton to restore walking. Walking is done at higher speed and with decreased metabolic energy cost and rate, and use of upper extremities only for balance and safety. The features of the system may be used in combination with an implantable FES system that integrates a sufficient number of stimulation channels and appropriate sensors.

### BRIEF DESCRIPTION OF DRAWINGS

FIG. 1A shows an exemplary smart clothing with both functional electrical system (FES) and exoskeleton movement.

FIG. 1B shows an exemplary motor positions and linkages for actuating leg movements.

FIG. 1C shows an exemplary motor for actuating body movements.

FIG. 1D shows an exemplary light projection system while FIG. 1E shows exemplary smart glasses to assist the user.

FIG. 2A shows an exemplary controller or processor for actuating body movements.

FIG. 2B shows an exemplary Internet of Things (IOT) for communications over the Internet.

FIG. 3A shows an exemplary foot control with FES.

FIG. 3B shows an exemplary foot control with FES.

FIG. 3C shows an exemplary stimulation waveform.

FIG. 4 shows an exemplary body control with FES and/or exoskeleton controls.

FIGS. 5, 6A and 6B collectively show an exemplary body movement control system.

FIGS. 7A-7C show exemplary processes for detecting musculature commands from the brain.

FIGS. 8A-8B shows systems for bowel and bladder control with FES.

### DESCRIPTION

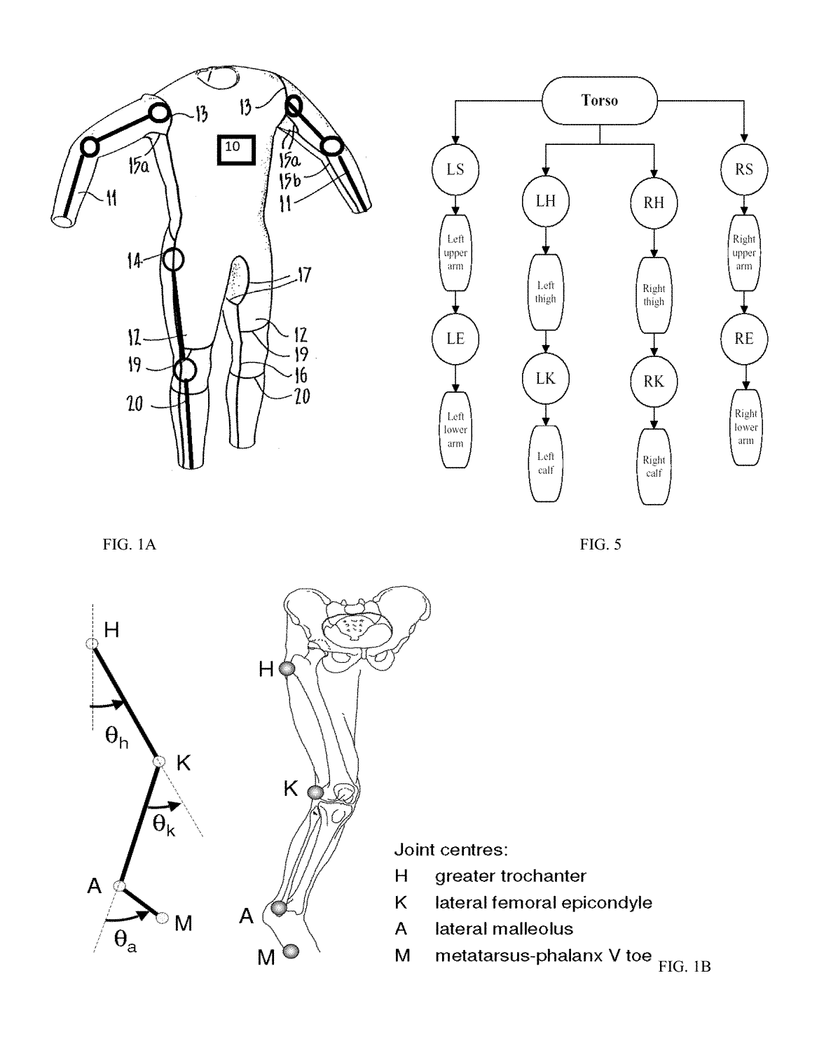

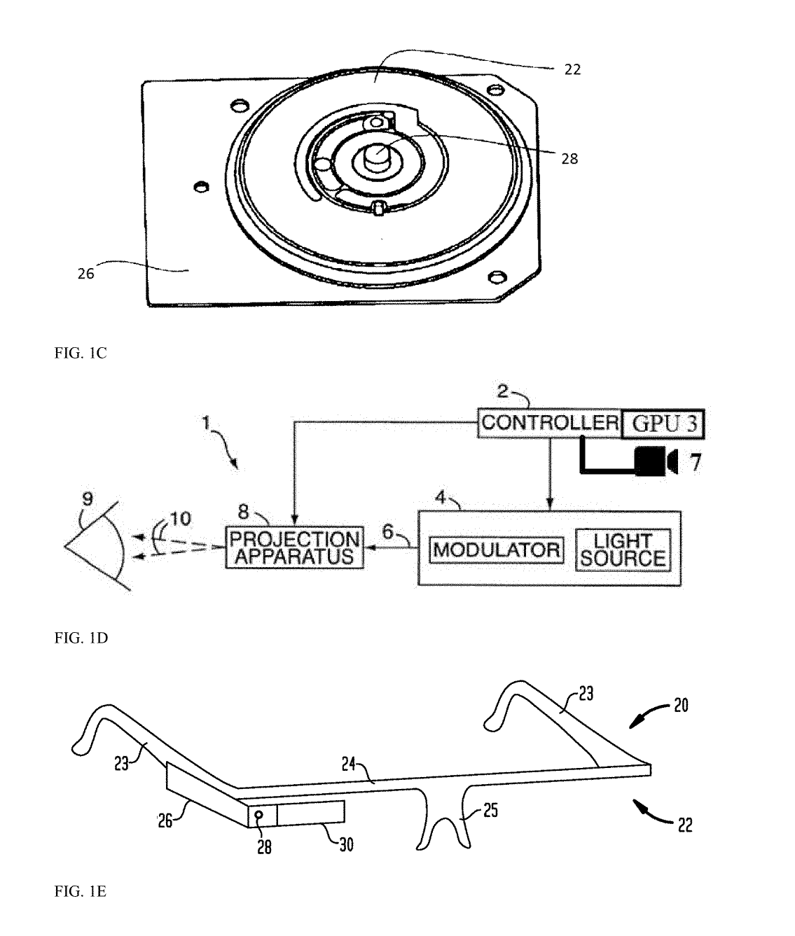

Referring to the drawings, the system, although applicable to all styles of body support structures, is particularly applicable to the type known as a smart wearable clothing shown in FIG. 1A, that is, having a main torso portion with controller 10, and full length arms and legs. Conductive threads 13 attach the arms 11 to the main torso with a controller 10 at the shoulder area, and actuators 14 extend down the lower portion of the length of the suit along the outer sides of the legs, with additional electrically conductive seams 15a connecting with the upper sections of the electrically conductive seam 14 to form underarm gussets which merge with electrically conductive seams 15b extending along the length of the arm. Electrically conductive seams 16 are also formed down the inside leg and merge with additional seams 17 to form a gusset at the crutch of the suit. Other electrically conductive seams include a back actuator 18 and series of linkages 11, 12 and 19 above and below the knee. Each conductive seam and actuator is connected to sensors using conductive fibers embedded in the clothing as detailed more below.

FIG. 1B shows an exemplary motor positions and linkages for actuating leg movements, while FIG. 1C shows an exemplary motor for moving the leg, among others. The flexible electronics in the clothing of FIG. 1A include a flat motor which is composed of a rotor and a stator, shown in FIG. 1C. A shaft 28 rotating in an axis of revolution is installed in the stator. The rotor is further composed of a rotor yoke 22 made by a soft magnetic material and a driving magnet in a ring shape that is mounted on the rotor yoke 22 by magnetic suction power of the driving magnet. A lower surface of the driving magnet towards the stator is magnetized with multipolar driving magnetic poles. In one embodiment, the shaft 28 is held by way of a bearing unit that is composed of the sintered oilless bearing and a thrust ball bearing to rotate freely. The stator yoke 26 can be mounted with the driving coil 24 and the sintered oilless bearing. The driving coils 24 is adhered on the stator yoke 26 in a concentric circle with centering the sintered oilless bearing. The stator yoke 26 can be formed as a printed circuit board as a substrate. The stator yoke 26 is formed with a bearing mounting section for staking the sintered oilless bearing as a circular plane with centering an axis of revolution. A driving coil mounting section for mounting the plurality of driving coils 24 is formed with surrounding the bearing mounting section, wherein a shape of the driving coil mounting section is approximately a part of surface of a circular cone shape having a vertex on the axis of revolution and is formed with inclining towards a direction away from the rotor in accordance with being far away from the axis. A conjugating section between the bearing mounting section 22 and the driving coil mounting section is formed as a pleat by bending the stator yoke 6 for plastic deformation. The conjugating section is formed by bending as far as a bent line and the conjugating section 24 is provided in a circle with respect to the axis of revolution of the stator yoke 26. On the other hand, a length “L” between the axis of revolution of the stator yoke 26. When the flat motor is assembled by using the stator yoke 26 mentioned above, the driving magnet draws the stator yoke 26 and results in deforming the stator yoke 26 to be approached to the driving magnet. While a rotary motor is shown, a linear motor can be used to move the linkages 11, 12 and 19, among others.

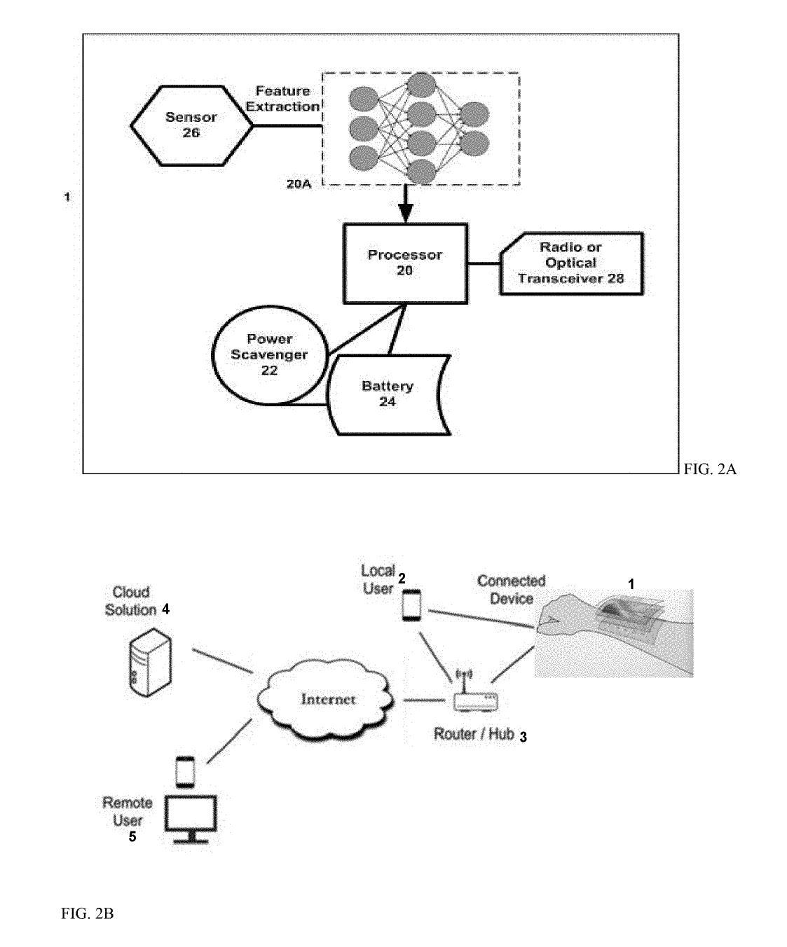

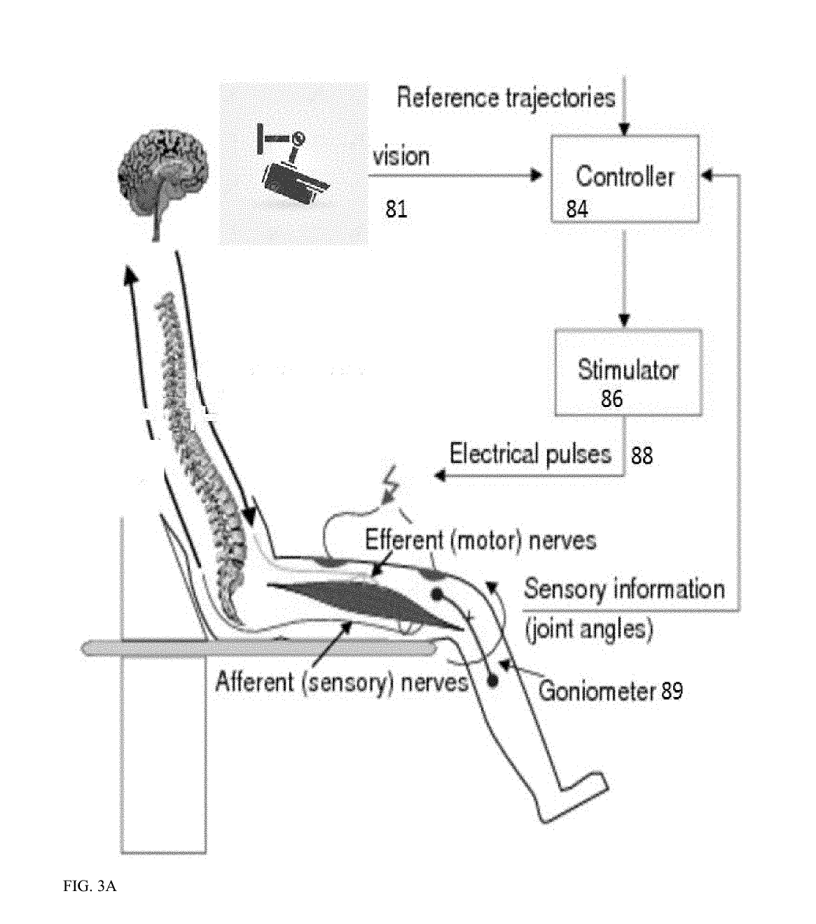

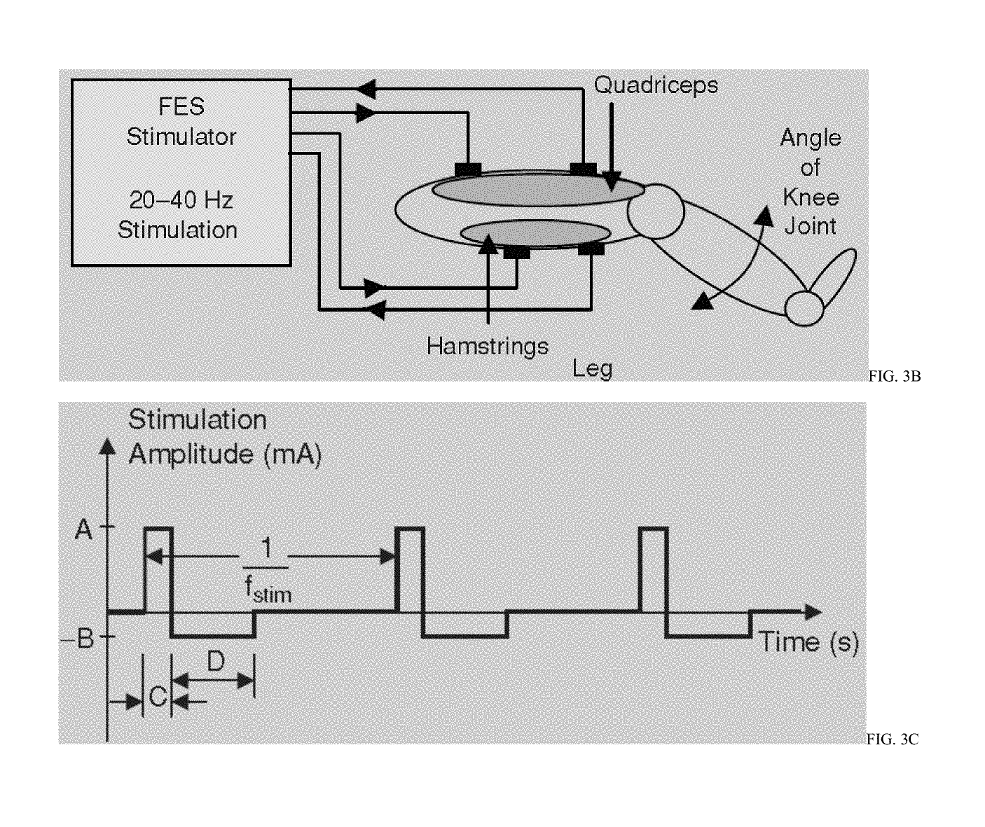

In one embodiment, the actuators form an exoskeleton that can help the user move or support weight/load for the user. Such suit can be used for improving the quality of life of persons who have, for example, lost the use of their legs, by providing assistive technology to enable system. Another area of application could be medical care, nursing in particular. The exoskeletons can help nurses lift and carry patients. The suit can be used in the military: decreased fatigue and increased productivity whilst unloading supplies or enabling a soldier to carry heavy objects while running or climbing stairs. Not only could a soldier potentially carry more weight, they could presumably wield heavier armor and weapons while lowering their metabolic rate or maintaining the same rate with more carry capacity. The actuators can be rotary motors, linear motors, or can be electroactive polymers (EAPs) which are polymers that exhibit a change in size or shape when stimulated by an electric field. The EAP can undergo a large amount of deformation while sustaining large forces as an artificial muscle. In one embodiment, the motors (indicated as circles on the clothing of FIG. 1) are connected to bars 11/12/20 and when the motors rotate, the joints are moved. The movement of the motors are controlled in an open feedback or closed feedback by the controller 10, an example of which is shown in FIG. 2A. The controller 10 can have a camera such as vision camera 81 (FIG. 3) to provide computer guidance in addition to the feedback from sensors and actuators.

Sensors and current drivers are positioned on the skin facing side of the body support of FIG. 1A. The sensors can include gyroscope, accelerometers, heart sensors, EKG (electrocardiogram) sensors, and EMG (electromyography) sensors. In natural muscle contraction, the force exerted by a muscle is dependent on the number of muscle fibers recruited and on the rate of action potentials imposed by the motor neurons on the active fibers. The muscle fibers are organized into motor units, which are groups of spatially dispersed fibers innervated by branches of the same motor axon. Variation in the strength of contraction is brought about by concurrent change in both recruitment and rate coding of motor units. The intensity of the electromyogram detected with large surface area electrodes is also influenced by both recruitment and rate coding such that a fixed (and practically linear) relationship exists between muscle force and EMG. Accordingly, the magnitude of the EMG provides an index of the active state of muscle, which in turn is related to its mechanical (force) output. The conversion of a predicted level of EMG into muscle activation in the present study involved delivery of current pulses through intramuscular electrodes. The sensors concurrently vary recruitment and rate coding, for example, by altering both the amplitude and frequency of the delivered current pulses, improve the reproduction of the active state of the muscle and thereby enhance the match between desired and evoked movements. One sensor type can be a neuro-sensor which deciphers the desired movement trajectory directly from ensembles of neurons in the cerebral cortex as neurons in the primary motor, premotor, and parietal cortices can be used to predict the intended direction of muscle motion.

An alternative mechanical signal is the pulsation of muscle resulting from the firing of a muscle's motor units. Such pulsations manifest on the skin surface as transverse vibrations, which can be easily detected using either pressure transducers or, more adequately, with accelerometers. Muscular vibration is called vibromyography (VMG) and can be used to measure muscular activity.

FIG. 1D shows an exemplary light projection system while FIG. 1E shows exemplary smart glasses to assist the user. FIG. 1D illustrates a system 1 for projecting an image onto a human retina and for scanning the eye. Using waveguides, 3D objects can be viewed from the projections. Data from the scan can be used for medical application such as glucose sensing or for emotion sensing when blood vessels dilate. The system 1 can be used in virtual reality applications, augmented reality applications, or a combination thereof.

The system 1 includes a controller 2 with graphical processing units (GPUs) 3 which generates signals in accordance with processes detailed hereinafter for presentation to a modulated optical source 4, which provides a modulated optical beam 6 to a projection apparatus 8. One or more cameras 7 can capture video images that can be projected by the projection apparatus 8 to stimulate the neurons on the eye of a blind person to enable the blind person to see at least a part of the video. The one or more cameras 7 can aim at the retina or can aim in the viewing direction of a user, depending on the application. The projection apparatus scans an image onto the retina of the eye 9 of a viewer, as indicated by reference numeral 10. The modulated light source includes a laser or other light source, which can be used for generation of an optical image. Preferably, the light source is a laser. The modulated light source can also include a discrete optical modulator, which is addressable and receives control signals from the controller 2. The optical modulator 4 can be of a known type, and is capable of modulating an optical beam with sufficient bandwidth to allow for presentation of the image to the viewer. Those skilled in the art will note that in certain embodiments, the light source may be modulated directly, without the inclusion of the discrete optical modulator. The on-chip laser light source emits a laser light beam that travels through the lens and a partially-silvered mirror. The laser light beam is reflected off a MEMS scanning mirror that is oscillating to provide a scan. The MEMS scanning mirror can be a resonate transducer, as known in the art. This device can be made to resonate at a desired frequency, either in one direction or in two directions. The resonate transducer may use a mechanically free beam of polysilicon and may also be positioned on thin membranes or diaphragms, cantilevers, and other flexure type mechanisms. The resonant frequency may be induced electronically by the scanner electronics, as known in the art. The MEMS scanning mirror has a mirrored surface and resonates at a controlled frequency in the horizontal and vertical direction, can produce a rastering scan pattern when a laser light source is reflected from its surface. The MEMS scanning mirror may be fabricated using integrated circuit techniques such as surface micromachining Alternative fabrication techniques also exist such as bulk micromachining, LIGA (a German acronym referring to lithography, electroforming, and injection molding), or LIGA-like machining, as known in the art. Additional fabrication techniques such as chemical-mechanical polishing may be performed to improve the optical quality of the mirrored surface by reducing the surface roughness.

The light emitting spots are produced by microns-sized diodes/lasers pumping phosphors located above the diodes/lasers. The individual spots of light used to make an image can all be of the same color (monochromatic) or of different colors. In the multiple color operation, the present invention uses a single or monochromatic pump source rather than discrete diode/laser sources of different colors. The lasers are fabricated in a two dimensional (“2D”) array format with established semiconductor processing techniques and practices. The 2D laser arrays are then integrated with nonlinear optical processes such as up-conversion (anti-Stokes process) in order to obtain multiple color outputs. Using photons with nonlinear up-conversion materials to obtain visible light output from a display device provides an advantage toward miniaturization of the present display. Using miniature (microns-sized) light emitting structures, such as surface emitting laser diodes, further allows for the miniaturization of the entire system concept illustrated in FIG. 1D to a much smaller system or package. The system uses two-dimensional arrays (m×n), of light emitting elements (photons), to miniaturize the display system to the generic manifestation. Miniaturization is also complemented through components and materials integrations in such areas as the use and integration of micro-lens array technology and the integration of the up-conversion (phosphor) materials directly onto the surfaces of the optics themselves.

The optical system is not limited to use of visible light, but may also employ light in other portions of the electromagnetic spectrum (e.g., infrared, ultraviolet) and/or may employ electromagnetic radiation that is outside the band of “light” (i.e., visible, UV, or IR), for example employing electromagnetic radiation or energy in the microwave or X-ray portions of the electromagnetic spectrum.

In some implementations, a scanning light display is used to couple light into a plurality of primary planar waveguides. The scanning light display can comprise a single light source that forms a single beam that is scanned over time to form an image. This scanned beam of light may be intensity-modulated to form pixels of different brightness levels. Alternatively, multiple light sources may be used to generate multiple beams of light, which are scanned either with a shared scanning element or with separate scanning elements to form imagery. These light sources may comprise different wavelengths, visible and/or non-visible, they may comprise different geometric points of origin (X, Y, or Z), they may enter the scanner(s) at different angles of incidence, and may create light that corresponds to different portions of one or more images (flat or volumetric, moving or static). The light may, for example, be scanned to form an image with a vibrating optical fiber.

A computer database of graphical imagery is addressed by graphics processing units (GPUs) 3 in the controller 2, such that each point (or pixel) along a sinusoidal path laser light represents an x-y pixel coordinate in the database so as to reconstruct a coherent image to a viewer and modulated to fit physiology of the eye. For example, since at a small distance from the fovea (FIG. 1C) the human eye has very poor ability to resolve sharpness or color, the system can supple the human visual cortex and brain's processing to integrate the entire visual field into a cohesive image. For example, the middle of the retina, where the minimum photon flux is presented due to the maximum velocity of the raster scan, is dimmer that the ends of the retina, which receive a maximum flux of photons. The prior art systems consequently must, at a minimum, compensate for the foregoing natural occurring phenomena to even the brightness of each of the pixels. Further, with a high concentration of image-sensing cones at the eye's fovea, the controller handles rapidly declining cone concentration as a function of distance from the fovea to the periphery of the retina. The controller drives the retinal illumination as center-weighted, which can be the inverse of the illumination. In one embodiment, illumination of just the central portion can be sufficient to create the desired image.

In the light source, a mirror oscillates sinusoidally, such as in an ellipsoidal pattern, which causes the formation of high-resolution imagery in a concentrated zone, while substantially lowering resolution in a circular field around that zone. By coupling the location of this zone of high resolution to the eye's foveal area via an eye tracking mechanism, as discussed below, a very high apparent resolution image is provided. System bandwidth requirements are reduced. Rather than a standard pixel grid in the horizontal and vertical axes, the computer can be tasked to generate pixels in an ellipsoidal sweep with a rotating central axis. This concentrates a large number of pixels into a central zone. The laser beam can be swept in sinusoidal patterns in such a manner that each sweep of the laser beam crosses at a single point in the x-y field, while the sweep precesses, so that a “frame” of image is represented by a circular field. The crossing point can be moved to any position within the field, via proper modulation of a mirror. As the laser beam is swept through a spiral pattern, it can be modulated in brightness and focus so that as the beam sweeps through the single point it is highly focused, yet much less bright. As the beam sweeps away from the point, it can grow grows brighter and less focused, so that the resultant circular field is of even apparent brightness. In this manner the beam crossing point 802 can be of extremely high resolution (since virtually every sweep passes through it) and of extremely high temporal information (since each sweep represents a small fraction of a “frame” representing one complete spiral sweep filling the entire circular field. For example, one complete spiral sweep of the circular field could occur in one-sixtieth ( 1/60th) of a second, and consist of 525 precessing sinusoidal sweeps; thus, the field could contain the same information as a field of NTSC video. In contrast to this focus point of all sweeps, the periphery of the field drops off in clarity and information responsive, such as in direct proportion, to the distance from the focus point. At the periphery of the field, resolution is low. Thus, the visual information of a frame (or field) of an image is more concentrated at the crossing point, and more diffuse at the periphery.

One embodiment uses a plurality of cameras 7 to provide a gesture control feature. A pair of light sources can be disposed to either side of cameras and controlled abyan image-analysis system. In some embodiments where the object of interest is a person's hand or body, use of infrared light can allow the motion-capture system to operate under a broad range of lighting conditions and can avoid various inconveniences or distractions that may be associated with directing visible light into the region where the person is moving. However, a particular wavelength or region of the electromagnetic spectrum is required.

It should be stressed that the foregoing arrangement is representative and not limiting. For example, lasers or other light sources can be used instead of LEDs. For laser setups, additional optics (e.g., a lens or diffuser) may be employed to widen the laser beam (and make its field of view similar to that of the cameras). Useful arrangements can also include short- and wide-angle illuminators for different ranges. Light sources are typically diffuse rather than specular point sources; for example, packaged LEDs with light-spreading encapsulation are suitable.

In operation, cameras 7 are oriented toward a region of interest in which an object of interest (in this example, a hand) and one or more background objects can be present. Light sources illuminate the region. In some embodiments, one or more of the light sources and cameras are disposed below the motion to be detected, e.g., where hand motion is to be detected, beneath the spatial region where that motion takes place. This is an optimal location because the amount of information recorded about the hand is proportional to the number of pixels it occupies in the camera images, the hand will occupy more pixels when the camera's angle with respect to the hand's “pointing direction” is as close to perpendicular as possible. Because it is uncomfortable for a user to orient his palm toward a screen, the optimal positions are either from the bottom looking up, from the top looking down (which requires a bridge) or from the screen bezel looking diagonally up or diagonally down. In scenarios looking up there is less likelihood of confusion with background objects (clutter on the user's desk, for example) and if it is directly looking up then there is little likelihood of confusion with other people out of the field of view (and also privacy is enhanced by not imaging faces). In this arrangement, image-analysis system can quickly and accurately distinguish object pixels from background pixels by applying a brightness threshold to each pixel. For example, pixel brightness in a CMOS sensor or similar device can be measured on a scale from 0.0 (dark) to 1.0 (fully saturated), with some number of gradations in between depending on the sensor design. The brightness encoded by the camera pixels scales standardly (linearly) with the luminance of the object, typically due to the deposited charge or diode voltages. In some embodiments, light sources 808, 810 are bright enough that reflected light from an object at distance rO produces a brightness level of 1.0 while an object at distance rB=2rO produces a brightness level of 0.25. Object pixels can thus be readily distinguished from background pixels based on brightness. Further, edges of the object can also be readily detected based on differences in brightness between adjacent pixels, allowing the position of the object within each image to be determined. Correlating object positions between images from cameras 7 allows image-analysis system to determine the location in 3D space of object 814, and analyzing sequences of images allows image-analysis system to reconstruct 3D motion of object using conventional motion algorithms.

In identifying the location of an object in an image according to an embodiment of the present invention, light sources are turned on. One or more images are captured using cameras. In some embodiments, one image from each camera is captured. In other embodiments, a sequence of images is captured from each camera. The images from the two cameras can be closely correlated in time (e.g., simultaneous to within a few milliseconds) so that correlated images from the two cameras can be used to determine the 3D location of the object. A threshold pixel brightness is applied to distinguish object pixels from background pixels. This can also include identifying locations of edges of the object based on transition points between background and object pixels. In some embodiments, each pixel is first classified as either object or background based on whether it exceeds the threshold brightness cutoff. Once the pixels are classified, edges can be detected by finding locations where background pixels are adjacent to object pixels. In some embodiments, to avoid noise artifacts, the regions of background and object pixels on either side of the edge may be required to have a certain minimum size (e.g., 2, 4 or 8 pixels).

In other embodiments, edges can be detected without first classifying pixels as object or background. For example, Δβ can be defined as the difference in brightness between adjacent pixels, and |Δβ| above a threshold can indicate a transition from background to object or from object to background between adjacent pixels. (The sign of Δβ can indicate the direction of the transition.) In some instances where the object's edge is actually in the middle of a pixel, there may be a pixel with an intermediate value at the boundary. This can be detected, e.g., by computing two brightness values for a pixel i: βL=(βi+βi−1)/2 and βR=(βi+βi+1)/2, where pixel (i−1) is to the left of pixel i and pixel (i+1) is to the right of pixel i. If pixel i is not near an edge, |βL-βR| will generally be close to zero; if pixel is near an edge, then |βL-βR| will be closer to 1, and a threshold on |βL-βR| can be used to detect edges.

In some instances, one part of an object may partially occlude another in an image; for example, in the case of a hand, a finger may partly occlude the palm or another finger Occlusion edges that occur where one part of the object partially occludes another can also be detected based on smaller but distinct changes in brightness once background pixels have been eliminated.

Detected edges can be used for numerous purposes. For example, as previously noted, the edges of the object as viewed by the two cameras can be used to determine an approximate location of the object in 3D space. The position of the object in a 2D plane transverse to the optical axis of the camera can be determined from a single image, and the offset (parallax) between the position of the object in time-correlated images from two different cameras can be used to determine the distance to the object if the spacing between the cameras is known.

Further, the position and shape of the object can be determined based on the locations of its edges in time-correlated images from two different cameras, and motion (including articulation) of the object can be determined from analysis of successive pairs of images. An object's motion and/or position is reconstructed using small amounts of information. For example, an outline of an object's shape, or silhouette, as seen from a particular vantage point can be used to define tangent lines to the object from that vantage point in various planes, referred to herein as “slices.” Using as few as two different vantage points, four (or more) tangent lines from the vantage points to the object can be obtained in a given slice. From these four (or more) tangent lines, it is possible to determine the position of the object in the slice and to approximate its cross-section in the slice, e.g., using one or more ellipses or other simple closed curves. As another example, locations of points on an object's surface in a particular slice can be determined directly (e.g., using a time-of-flight camera), and the position and shape of a cross-section of the object in the slice can be approximated by fitting an ellipse or other simple closed curve to the points. Positions and cross-sections determined for different slices can be correlated to construct a 3D model of the object, including its position and shape. A succession of images can be analyzed using the same technique to model motion of the object. Motion of a complex object that has multiple separately articulating members (e.g., a human hand) can be modeled using these techniques.

More particularly, an ellipse in the xy plane can be characterized by five parameters: the x and y coordinates of the center (xC, yC), the semimajor axis, the semiminor axis, and a rotation angle (e.g., angle of the semimajor axis relative to the x axis). With only four tangents, the ellipse is underdetermined. However, an efficient process for estimating the ellipse in spite of this fact involves making an initial working assumption (or “guess”) as to one of the parameters and revisiting the assumption as additional information is gathered during the analysis. This additional information can include, for example, physical constraints based on properties of the cameras and/or the object. In some circumstances, more than four tangents to an object may be available for some or all of the slices, e.g., because more than two vantage points are available. An elliptical cross-section can still be determined, and the process in some instances is somewhat simplified as there is no need to assume a parameter value. In some instances, the additional tangents may create additional complexity. In some circumstances, fewer than four tangents to an object may be available for some or all of the slices, e.g., because an edge of the object is out of range of the field of view of one camera or because an edge was not detected. A slice with three tangents can be analyzed. For example, using two parameters from an ellipse fit to an adjacent slice (e.g., a slice that had at least four tangents), the system of equations for the ellipse and three tangents is sufficiently determined that it can be solved. As another option, a circle can be fit to the three tangents; defining a circle in a plane requires only three parameters (the center coordinates and the radius), so three tangents suffice to fit a circle. Slices with fewer than three tangents can be discarded or combined with adjacent slices.

To determine geometrically whether an object corresponds to an object of interest comprises, one approach is to look for continuous volumes of ellipses that define an object and discard object segments geometrically inconsistent with the ellipse-based definition of the object—e.g., segments that are too cylindrical or too straight or too thin or too small or too far away—and discarding these. If a sufficient number of ellipses remain to characterize the object and it conforms to the object of interest, it is so identified, and may be tracked from frame to frame.

In some embodiments, each of a number of slices is analyzed separately to determine the size and location of an elliptical cross-section of the object in that slice. This provides an initial 3D model (specifically, a stack of elliptical cross-sections), which can be refined by correlating the cross-sections across different slices. For example, it is expected that an object's surface will have continuity, and discontinuous ellipses can accordingly be discounted. Further refinement can be obtained by correlating the 3D model with itself across time. An object of interest can be brightly illuminated during the times when images are being captured. In some embodiments, the silhouettes of an object are extracted from one or more images of the object that reveal information about the object as seen from different vantage points. While silhouettes can be obtained using a number of different techniques, in some embodiments, the silhouettes are obtained by using cameras to capture images of the object and analyzing the images to detect object edges.

The system can include EKG, EEG and EMG sensors on the eye, along with temperature sensors. The system also includes impedance sensors for bioelectrical impedance analysis (BIA) in estimating body composition, and in particular body fat. BIA determines the electrical impedance, or opposition to the flow of an electric current through body tissues which can then be used to calculate an estimate of total body water (TBW). TBW can be used to estimate fat-free body mass and, by difference with body weight, body fat.

In one embodiment, micro-acoustic elements (piezoelectric elements) may be placed insider or on a surface of the lens to transmit audible signals through bone resonance through the skull and to the cochlea. In other embodiments, the audible signals transmitted to the user using the micro-acoustic elements may be transmitted. High quality audio can be sent for secure voice communication or for listening to music/video. The audio can be an alarm or warning as well, for example, when the cardiac rhythm is determined to be outside a predetermined threshold based on monitored changes of the retinal vascularization. For example, the audible signal may be a recommended action and/or warning based on cardiac rhythm or an abnormal condition.

In one embodiment, a contact lens can be used to project images into the retina. In this embodiment, a clear solar cell layer in the 3D chip can be used to generate power. In another embodiment, when not displaying, a bank of parallel LEDs can be used to generate electric power from light. The LED PN junctions are photovoltaic. While solar cells are made with a large area PN junction, a LED has only a small surface area and is not as efficient. Deposited on the same layer as the TFT and above the pixel electrode is a transparent power-generating element. In one embodiment, the power generating element is a semiconductor layer made using an amorphous silicon (a-Si) photodiode that generates photoelectric current. In this embodiment, light is absorbed only from the surface and in areas where the LCD is ‘off’ using an antireflective coating. The coating transforms the device into a 2-way mirror. The anti-reflective coating can consist of the amorphous silicon layer itself. By carefully choosing the layer thicknesses, light coming from under the silicon layer will be transmitted, while sunlight will be reflected and diffused back into the amorphous silicon layer. Another embodiment uses a photoactive, doped liquid crystal such as crystals with titanyl phthalocyanines (TiOPc). TiOPc is quite suitable as a photoreceptive material for liquid crystal diodes because TiOPc has sufficient light sensitivity at long wavelength region between 600 nm to 850 nm. The crystal can be formed by distributing a charge generation material (CGM) in a resin, among others. As for such CGM, for example, inorganic photoconductive materials such as selenium or alloys thereof, CdS, CdSe, CdSSe, ZnO, ZnS, metal or non-metal phthalocyanine compounds; azo compounds such as bisazo compounds, trisazo compounds, such as squarium compounds, azurenium compounds, perylene compounds, indigo compounds, quinacridone compounds, polyquinone-type compounds, cyanine dyes, xanthene dyes and transportation complexes composed of poly-N-carbazoles and trinitrofluorenone can be used. These compounds may be used either individually or two or more kinds in combination. The crystal becomes opaque when the electrodes force a voltage across it, meaning that photons are captured inside the crystal. The photons generate electron-hole pairs similar to those in a solar cell. The generated electron hole pairs flow to the biasing terminals, effectively supplying power. In this embodiment, no special coatings are needed, since light does not need to be preferentially reflected, and is only absorbed in pixels that are intentionally opaque. The pixel addressing circuitry turns pixel into an opaque state by applying a voltage across the pixel. This pixel can then capture all light impinging upon it from both the backlight and top without affecting the contrast of the display. In this embodiment, the more photons absorbed in the dark pixels, the higher the resulting contrast. Thus, by using appropriate photoactive liquid crystal, a portion of the absorbed photons can be turned into usable electrical power.

One embodiment harvests electricity from WiFi signals or cellular signals that are omnipresent. An ultra-wideband rectifying antenna used to convert microwave energy to DC power. This energy-scavenging antenna can be formed as part of a 3D chip with sensors, antennas and super-capacitors onto paper or flexible polymers. The antenna can generate energy from WiFi, cellular signal, radio signal, and TV signals as well as electric power lines.

Biosensor(s) on the active contact lens allows, for example, continuous sampling of the interstitial fluid on a user's cornea. This fluid is in indirect contact with blood serum via the capillaries in the structure of the eye and contains many of the markers that are used in blood analysis to determine a person's health condition. The sampling and analysis of this fluid allows for continuous assessment of a user's fatigue level and early detection of infectious components without taking a blood sample. The same interstitial fluid can be used to assess the user's blood glucose level allowing for continuous glucose monitoring without blood sampling for diabetic patients.

The system can use a bio-battery or an energy storing device that is powered by organic compounds, usually being glucose, such as the glucose in human blood. When enzymes in human bodies break down glucose, several electrons and protons are released. Therefore, by using enzymes to break down glucose, bio-batteries directly receive energy from glucose. These batteries then store this energy for present or later use. Like any cell battery, bio-batteries contain an anode, cathode, separator and electrolyte with each component layered on top of another in a 3D structure that is deposited on the plastic 3D chip. Between the anode and the cathode lies the electrolyte which contains a separator. The main function of the separator is to keep the cathode and anode separated, to avoid electrical short circuits. This system as a whole, allows for a flow of protons (H+) and electrons (e−) which ultimately generates electricity. Bio batteries are based on the amount of glucose available. The body decomposes materials to glucose (if they are not already in the proper stage) is the main step in getting the cycle started. Materials can be converted into glucose through the process of enzymatic hydrolysis. Enzymatic hydrolysis is the process in which cellulose (an insoluble substance) is converted to glucose by the addition of enzymes. Once glucose is present, oxygen and other enzymes can act on it to further produce protons and electrons. The bio-batteries use enzymes to convert glucose into energy. When glucose first enters the battery, it enters through the anode. In the anode the sugar is broken down, producing both electrons and protons : Glucose→Gluconolactone+2H++2e−. These electrons and protons produced now play an important role in creating energy. They travel through the electrolyte, where the separator redirects electrons to go through the mediator to get to the cathode.[1] On the other hand, protons are redirected to go through the separator to get to the cathode side of the battery. The cathode then consists of an oxidation reduction reaction. This reaction uses the protons and electrons, with the addition of oxygen gas, to produce water: O2+4H++4e−→2H2O. There is a flow created from the anode to the cathode which is what generates the electricity in the bio-battery. The flow of electrons and protons in the system are what create this generation of electricity.

Components of the system may also be included in a 3D plastic chip with a plurality of layers of components fabricated thereon. In some embodiments, the retinal vascularization monitoring components may be attached as a portion of a layer. In some of the embodiments discussed herein, the battery elements may be fabricated as part of the 3D IC device, or may be included as elements in a stacked layer. It may be noted as well that other embodiments may be possible where the battery elements are located externally to the stacked integrated component layers. Still further diversity in embodiments may derive from the fact that a separate battery or other energization component may also exist within the media insert, or alternatively these separate energization components may also be located externally to the media insert. In some embodiments, these media inserts with 3D IC layers may assume the entire annular shape of the media insert. Alternatively in some cases, the media insert may be an annulus whereas the stacked integrated components may occupy just a portion of the volume within the entire shape.

The active contact lens system allows for full situational awareness and mobility by enabling real-time information display to permit quick decision-making. The potential applications in gaming, virtual reality, and training are innumerable, and will be readily apparent to persons of skill in these arts.

FIG. 1E illustrates an example system 20 for receiving, transmitting, and displaying data. The system 20 is shown in the form of a wearable computing device eyeglass 22. The wearable computing device 22 may include side-arms 23, a center frame support 24, and a bridge portion with nosepiece 25. In the example shown in FIG. 1D, the center frame support 24 connects the side-arms 23. The wearable computing device 22 does not include lens-frames containing lens elements. The wearable computing device 22 may additionally include an onboard computing system 26 and a video camera 28.

The wearable computing device 22 may include a single lens element 30 that may be coupled to one of the side-arms 23 or the center frame support 24. The lens element 30 may include a display such as the laser projector described above, and may be configured to overlay computer-generated graphics upon the user's view of the physical world. In one example, the single lens element 30 may be coupled to the inner side (i.e., the side exposed to a portion of a user's head when worn by the user) of the extending side-arm 23. The single lens element 30 may be positioned in front of or proximate to a user's eye when the wearable computing device 22 is worn by a user. For example, the single lens element 30 may be positioned below the center frame support 24.

FIG. 2A shows an exemplary printed Internet of Things (IoT) flexible sensor device 1. The flexible sensor device 1 can have a flexible substrate 23 with a surface that is configured for receiving a flexible sensor 26. The flexible sensor 26 can be any flexible sensor or sensor circuit that can detect the presence of a target substance (a chemical compound) or electrical pattern (such as EKG or DNA, for example) or any other suitable tests. The substrate 23 can be made of a polymeric body and/or an inorganic-organic complex. Also, ceramics with suitable flexibility can be included in the substrate, as detailed below. The device is printed using low cost roll-to-roll manufacturing, inkjet printing or plasma jet fabrication, or a combination thereof, among others. In a complex sensor circuit, the device 1 can have a flexible substrate that is configured for receiving a first flexible sensor circuit electronically coupled to a second flexible sensor circuit. Such electronic coupling can be obtained, for example, an electronic path operatively linking a first flexible sensor circuit and a second flexible sensor circuit. The electronic coupling of flexible sensor circuits can be used to prepare more complex sensor systems. Also, any number of sensor circuits can be electronically coupled. The sensor circuits can be configured as described herein. In other embodiments, hybrid flexible electronics with part flexible circuit and part conventional circuits can be implemented.

One or more structures printed on the device can be a sensor 26 which captures information from the environment, such as temperature, EKG, DNA information, or glucose level, for example. The sensor can be a combination of sensors, nanowires, conductive polymers, and the like, and can include target recognition moieties for detecting target substances. While the raw data can be sent directly over the Internet via a wired or wireless connection, in one embodiment, the data is provided to an optional input pre-processor and then to a feature extractor/processor 20 which transforms raw data into a set of features to increase detection and minimize data transmission size/power consumption. The processor can be a conventional IC mounted on a printed motherboard, or the processor can be directly printed on the substrate. In one embodiment, the processor contains a general purpose processor communicating with a neural network 20A that can be trained to recognize patterns. The neural network 20A can have analog or digital implementations. In one embodiment, a pattern-matching recognition neural network is composed of 128 arithmetic units or neurons to perform two types of pattern recognition; the k-nearest neighbour (KNN) recognition and the radial basis function. Various desired patterns can be programmed and engine returns a positive match, uncertain, or negative match within a fixed time. The network is used as part of a wake-up system so that a sensor subsystem can pass a series of feature vectors to the neural network, which matches it against a stored dataset. If a wake up event is detect, the processor 20 is woken to decide whether to process information locally or to send information on to a sensor hub.

The sensor and processor 20 is powered by a power scavenger 22, an energy storage device 24, or a combination thereof. The scavenger 22 can be a printed antenna harvesting energy from FM stations, WiFi routers, cellular stations in one embodiment. The scavenger 22 can capture heat, sound, wind, or solar energy in other embodiments. The energy storage device 24 can be a printed supercapacitor or printed battery, among others.

The flexible substrate 23 can have any suitable shape or dimension along any vector. The flexible substrate 23 can also be a porous substrate. The pores (not shown) can extend, for example, from the surface into the substrate 23 or all the way through the substrate 23. Non-limiting examples of the shape of the substrate 23 can include a rectangle, block, triangle, amorphous shape, sphere, cube, polygon, and the like formed in three dimensions or as a substantially two dimensional sheet. The substrate can be any substrate known in the art.

FIG. 2B shows an exemplary cloud-based structure supporting sensors of FIG. 2A. A connected flexible printed device 1 such as the sensor of FIG. 2A is connected (wired or wireless) to a router/hub 3. The router/hub 3 transmits to the Internet to a cloud solution 4 which can provide storage of data flowing from the connected sensor of FIG. 2A, or can include complex analytic functions that are performed on the data coming from the device and reported to a local user 2 or remote user 5. The local user 5 can interact directly with the sensor device 1 to either control it, or receive information regarding its operation. The router connects the device 1 to the Internet with a suitable modem using fiber optic, ADSL, cable, cellular, among others. The remote user 5 is not in the proximity of the device and can control or receive information regarding the device from afar. One embodiment sends data to the Cloud using NFC or Bluetooth and then use the local user's smartphone as their hub to the Internet, or a special hub can be provided that routes the Bluetooth data through Ethernet/Wi-Fi/cellular to the Internet. Wi-Fi, a more power-hungry solution, but still relatively low power, can be used for devices that are connected to external power, or can be charged periodically. Wi-Fi, in contrast to Bluetooth, can connect to the Internet and the Cloud directly via an existing Wi-Fi router without a special hub required.If Ethernet (LAN) is available where the device is located and the device is stationary, a wired connection may be a good choice—it is usually the lowest cost and simplest connectivity method for the device.

Electrically functional inks are deposited on the substrate, creating active or passive devices, such as thin film circuits, sensors, transistors or resistors. The term printed electronics specifies the process and can utilize any solution-based material. The use of flexible electronic printing enables low-cost volume fabrication which has opened the door for the medical industry to include electrically functional parts as disposables. Printed electronics offer reliability as well as patient comfort, less invasiveness and can be disposable, with the ability to offer remote diagnostics in a cost effective, disposable form is driving use of printed electronics. Biosensors such as EKG/ECG electrodes, glucose test strips and pads for drug delivery manufactured by using combinations of silver, silver-silver chloride, carbon, and di-electric inks printed on thin film polyester can be used. Also, drivers and output pads for FES devices can be formed on the substrate for hand control using FES. IN one embodiment, electrical pulses, 14 channels, and three levels of electrical stimulations control the user's hand. The impulses generated are transmitted to the muscles that are to be stimulated through electrode pads fastened to the skin. An electrical stimulus is applied to the user's forearm because most of the muscles that control the fingers and the wrist are located the forearm. For this purpose, a forearm contact region on the suit of FIG. 1 is used. The electrical stimuli are generated by an electronic pulse generator and transmitted via the electrode pads. The pads on the suit contact the upper and lower parts of the user's forearm. At least, five channels are needed to stimulate the muscles that are used to bend finger-joints, and two additional channels are needed to stimulate the muscles that are used to bend finger extensions and cause wrist flexions. The system stimulates seven muscles (the superficial flexor muscle, deep flexor muscle, long flexor muscle of the thumb, common digital extensor muscle, flexor carpi radialis muscle, long palmar muscle, and flexor carpi ulnaris muscle).

FIG. 3A shows an exemplary computer controlled actuation system using EMG and Functional Electrical Stimulation (FES) for knee joint angle control. The system includes a computer vision system 81 communicating with a controller 84 which drives an FES stimulator 86 generating electrical pulses 88 to cause the motor nerves to move by controlling efferent and afferent nerves. The computer vision system automatically generates 3D models of objects and can control the foot movements navigate around an obstacle automatically.

A sensor 89 such as goniometer 89 can be used to detect joint angles. The electrical nerve stimulation generates contractions of weakened or paralyzed muscles. In combination with sensors and feedback control, the system of FIG. 3 can elicit functional movements, such as walking and cycling, and to restore certain motor functions. The system can provide temporary assistance, e.g., during relearning of gait, or permanent replacement of lost motor functions (neuro-prosthesis). The system improves muscle size and strength, increases the range of joint motion and improves cardiopulmonary fitness by providing significant training effects. FIG. 3 also provides control of the knee joint angle by quadriceps stimulation. The knee joint angle is measured and fed back to the controller 84, which generates a suitable stimulation pattern to achieve tracking of a reference trajectory. Stimulation can either be applied directly to the peripheral motor nerves (as shown in FIG. 2) or to the sensory nerves (neuro-modulation). The latter causes an indirect stimulation of motor nerves while ensuring the natural inhibition of antagonistic muscles. A general problem with FES is rapid muscle fatigue. External stimuli, which replace the missing commands from the central nervous system, tend to invert the recruitment order of muscle fibers: motor neurons with larger diameter are activated first as they have a lower threshold; they recruit the faster and more powerful (type 2 or white) fibers, which fatigue more quickly than the slower and less powerful (type 1 or red) muscle fibers. Electrical stimulation is realized by attaching surface electrodes to the skin, allowing for Class 1 certification with the FDA.

The angle of a joint, or the torque about that joint, can be regulated by varying the tension produced in the flexor and extensor muscles that actuate the joint. For the knee joint, the flexor muscles are the hamstrings group, while the extensor muscles are the quadriceps group. The hamstrings flex the knee to a bent position, while the quadriceps extend the knee and straighten the leg. A biological neurological system produces tetanic contractions, which are characterized by sustained, constant tension, by stimulating each motor unit at a frequency of 6-8 Hz. Adjacent motor units are stimulated sequentially so that the overall muscle produces a tetanic contraction. If the muscle tension produced by the tetanic contraction is sufficiently high, the knee angle changes, as shown. In FIG. 3B, the FES system can produce tetanic contractions in a spinal cord injured subject. However, the system must stimulate at 20-40 Hz to achieve this result because the individual motor units cannot be stimulated sequentially with FES. FIG. 3C shows an exemplary stimulation pulse train. A typical stimulation waveform used for transcutaneous FES is a biphasic square-wave pulse train with a frequency of 20-40 Hz, an amplitude of 0-120 mA, and a pulse duration of 0-300 μs. A biphasic waveform is used because it induces charge transfer into the tissue and then immediately induces charge transfer out of the tissue. This pattern of charge transfer prevents galvanic processes that can cause tissue damage [18]. Notice that the amount of charge transferred into the tissue (given by the product AC) is the same as the charge transferred out of the tissue (given by the product BD).

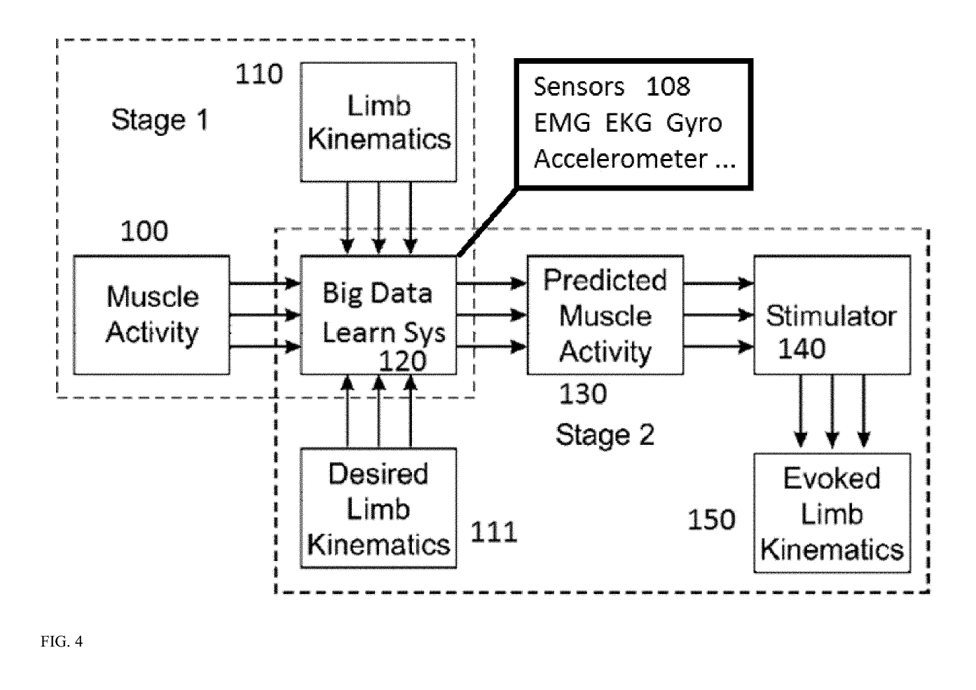

FIG. 4 shows an exemplary limb control system. The system is a big data learning system and operates in two phases or stages. In a first stage, muscle activity 100 and limb kinematics 110 data are captured by sensors 108 and correlated to The stimulation parameters such as maximum pulse amplitude (0-150 V), pulse width (50-800 μs) and pulse frequency (5-60 Hz) are selected independently for each channel Stimulation pulses are monophasic and of rectangular shape. Because certain locomotor activities require modulated stimulation output, the system generates amplitude modulated pulses. The modulation signal is part of the stimulation pattern description. With variable stimulation frequency, one can achieve an appropriate compromise between fatigue and force with the frequency of each stimulation channel is set-up independently. The safety measures are of utmost importance. The stimulation channels are voltage sources, thus reducing the possibility of skin burn in case of poor electrode-skin contact. All stimulation outputs are mutually doubly electrically isolated to prevent leakage currents between the electrodes. Each stimulation output is also protected against DC current. A battery monitor buzzes in case the batteries are low. electrical activities captured by sensors using a learning system 120. The learning system 120 learns about the electrical activities and maps the muscle activity and limb kinematics to desired limb kinematics 111. During a second phase or stage, the learning system 120 predicts desired muscle activities required for a task in 130 and converts such muscle activities to electrical signals sent to a stimulator 140 which drives evoked limb kinematics 150.

In the second stage, the probabilistic relationship between muscle activity and kinematics identified in the first stage is used to predict muscle activity associated with a new set of intended or desired movements of the muscle such as the finger or leg. The predicted patterns of muscle activity are transformed into frequency-modulated trains of pulses that are used to control a set of muscle stimulators to evoke finger movements in other subjects.

In one embodiment for analyzing finger movements, the sensors can be flexible strain gauge transducers to record joint angles from the metacarpalphalangal (MCP) joint, the proximal interphalangeal (PIP) joint, and the distal interphlangeal (DIP) joint of the third digit. Surface electrodes (Ag-AgCl, 4 mm diameter) are attached to the skin over the distal radius served as reference electrodes. In one embodiment, EMG signals are amplified with a gain of 1000, bandpass filtered (30-1000 Hz), and digitally sampled at 2000 Hz. Training movements are captured and used for subsequent training of the learning machine to yield the probabilistic relationships between muscle activity and joint kinematics. The movements were designed to cover much of the joint space associated with relatively natural movements. The duration of the training set was 60 sec. Segments were extracted that were used to represent different types of desired movements, for example, in analyzing the hand, the movements include: tapping, pushing, pulling, transition from pushing to tapping movements, and transition from tapping into pulling movements. EMG data were collected during the desired movements and used for comparison with the predicted patterns of EMG. The training EMG signals are full-wave rectified and low-pass filtered at 2 Hz. Joint angular velocities were calculated for each joint by digital differentiation of the joint angle data. Positive values for joint angular velocity indicated flexion movements, whereas negative values indicated extension movements. Joint angle, joint angular velocity, and EMG signals were all resampled at 200 Hz/signal. EMG magnitude was normalized to a percentage of the peak EMG within the training set and rounded to the nearest 1% increment. Joint angles and joint angular velocities were rounded into intervals of 1° and 1°/sec, respectively.

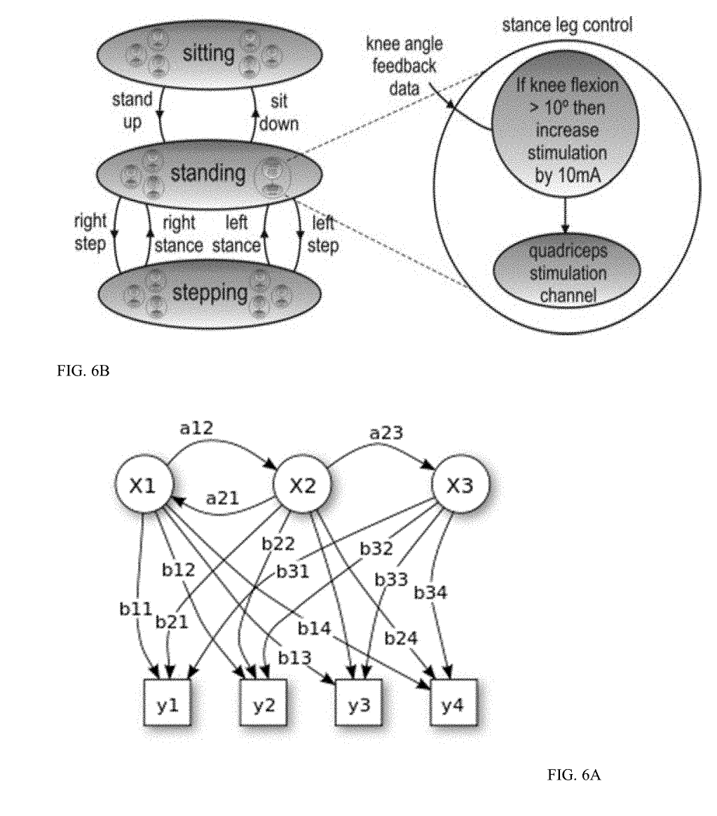

FIGS. 5, 6A and 6B collectively show an exemplary body movement control system. Human movement involves a periodic motion of the legs. Regular walking involves the coordination of motion at the hip, knee and ankle, which consist of complex joints. The muscular groups attached at various locations along the skeletal structure often have multiple functions. The majority of energy expended during walking is for vertical motion of the body. When a body is in contact with the ground, the downward force due to gravity is reflected back to the body as a reaction to the force. When a person stands still, this ground reaction force is equal to the person's weight multiplied by gravitational acceleration. Forces can act in other directions. For example, when we walk, we also produce friction forces on the ground. When the foot hits the ground at a heel strike, the friction between the heel and the ground causes a friction force in the horizontal plane to act backwards against the foot. This force therefore causes a breaking action on the body and slows it down. Not only do people accelerate and brake while walking, they also climb and dive. Since reaction force is mass times acceleration, any such acceleration of the body will be reflected in a reaction when at least one foot is on the ground. An upwards acceleration will be reflected in an increase in the vertical load recorded, while a downwards acceleration will be reduce the effective body weight.

In the suit of FIG. 1A, sensors with tri-axial accelerometers are formed on the suit at different body locations for recording, for example the tree structure of FIG. 5. As shown therein, sensors can be placed on the four branches of the links connect to the root node (torso) with the connected joint, left shoulder (LS), right shoulder (RS), left hip (LH), and right hip (RH). Furthermore, the left elbow (LE), right elbow (RE), left knee (LK), and right knee (RK) connect the upper and the lower extremities. The wireless monitoring devices can also be placed on upper back body near the neck, mid back near the waist, and at the front of the right leg near the ankle, among others.

The sequence of human motions can be classified into several groups of similar postures and represented by mathematical models called model-states. A model-state contains the extracted features of body signatures and other associated characteristics of body signatures. Moreover, a posture graph is used to depict the inter-relationships among all the model-states, defined as PG(ND,LK), where ND is a finite set of nodes and LK is a set of directional connections between every two nodes. The directional connection links are called posture links. Each node represents one model-state, and each link indicates a transition between two model-states. In the posture graph, each node may have posture links pointing to itself or the other nodes.

In one implementation shown in FIG. 6A, a hidden markov model (HMM) is used to track patient motor skills or patient movement patterns. FIG. 6B shows an exemplary HMM with states for sitting, standing and stepping with a one stance leg control rule within the standing state. In this example, based on knee angle feedback, stimulation amplitude over quadriceps is increased by 10 mA when knee become flexed more than 10°. In the pre-processing phase, the system obtains the human body profile and the body signatures to produce feature vectors. In the model construction phase, the system generate a posture graph, examine features from body signatures to construct the model parameters of HMM, and analyze human body contours to generate the model parameters. In the motion analysis phase, the system uses features extracted from the body signature sequence and then applies the pre-trained HMM to find the posture transition path, which can be used to recognize the motion type. Then, a motion characteristic curve generation procedure computes the motion parameters and produces the motion characteristic curves.

In one embodiment, big data analyzers may be used to track the patient's daily movement and living pattern. These data driven analyzers may incorporate a number of models such as parametric statistical models, non-parametric statistical models, clustering models, nearest neighbor models, regression methods, and engineered (artificial) neural networks. Prior to operation, data driven analyzers or models of the patient stoke patterns are built using one or more training sessions. The data used to build the analyzer or model in these sessions are typically referred to as training data. As data driven analyzers are developed by examining only training examples, the selection of the training data can significantly affect the accuracy and the learning speed of the data driven analyzer. One approach used heretofore generates a separate data set referred to as a test set for training purposes. The test set is used to avoid overfitting the model or analyzer to the training data. Overfitting refers to the situation where the analyzer has memorized the training data so well that it fails to fit or categorize unseen data. Typically, during the construction of the analyzer or model, the analyzer's performance is tested against the test set. The selection of the analyzer or model parameters is performed iteratively until the performance of the analyzer in classifying the test set reaches an optimal point. At this point, the training process is completed. An alternative to using an independent training and test set is to use a methodology called cross-validation. Cross-validation can be used to determine parameter values for a parametric analyzer or model for a non-parametric analyzer. In cross-validation, a single training data set is selected. Next, a number of different analyzers or models are built by presenting different parts of the training data as test sets to the analyzers in an iterative process. The parameter or model structure is then determined on the basis of the combined performance of all models or analyzers. Under the cross-validation approach, the analyzer or model is typically retrained with data using the determined optimal model structure.

In general, multiple dimensions of a user's EEG, EKG, BI, ultrasound, optical, acoustic, electromagnetic, or electrical parameters are encoded as distinct dimensions in a database. A predictive model, including time series models such as those employing autoregression analysis and other standard time series methods, dynamic Bayesian networks and Continuous Time Bayesian Networks, or temporal Bayesian-network representation and reasoning methodology, is built, and then the model, in conjunction with a specific query makes target inferences. Bayesian networks provide not only a graphical, easily interpretable alternative language for expressing background knowledge, but they also provide an inference mechanism; that is, the probability of arbitrary events can be calculated from the model. Intuitively, given a Bayesian network, the task of mining interesting unexpected patterns can be rephrased as discovering item sets in the data which are much more—or much less—frequent than the background knowledge suggests. These cases are provided to a learning and inference subsystem, which constructs a Bayesian network that is tailored for a target prediction. The Bayesian network is used to build a cumulative distribution over events of interest.

In another embodiment, a genetic algorithm (GA) search technique can be used to find approximate solutions to identifying the user daily pattern. Genetic algorithms are a particular class of evolutionary algorithms that use techniques inspired by evolutionary biology such as inheritance, mutation, natural selection, and recombination (or crossover). Genetic algorithms are typically implemented as a computer simulation in which a population of abstract representations (called chromosomes) of candidate solutions (called individuals) to an optimization problem evolves toward better solutions. Traditionally, solutions are represented in binary as strings of 0s and 1s, but different encodings are also possible. The evolution starts from a population of completely random individuals and happens in generations. In each generation, the fitness of the whole population is evaluated, multiple individuals are stochastically selected from the current population (based on their fitness), modified (mutated or recombined) to form a new population, which becomes current in the next iteration of the algorithm. Substantially any type of learning system or process may be employed to determine the daily living patterns so that unusual events can be flagged.

In one embodiment, clustering operations are performed to detect patterns in the data. In another embodiment, a neural network is used to recognize each pattern as the neural network is quite robust at recognizing user habits or patterns. Once the treatment features have been characterized, the neural network then compares the input user information with stored templates of treatment vocabulary known by the neural network recognizer, among others. The recognition models can include a Hidden Markov Model (HMM), a dynamic programming model, a neural network, a fuzzy logic, or a template matcher, among others. These models may be used singly or in combination.

Dynamic programming considers all possible points within the permitted domain for each value of i. Because the best path from the current point to the next point is independent of what happens beyond that point. Thus, the total cost of [i(k), j(k)] is the cost of the point itself plus the cost of the minimum path to it. Preferably, the values of the predecessors can be kept in an MxN array, and the accumulated cost kept in a 2×N array to contain the accumulated costs of the immediately preceding column and the current column. However, this method requires significant computing resources. For the recognizer to find the optimal time alignment between a sequence of frames and a sequence of node models, it must compare most frames against a plurality of node models. One method of reducing the amount of computation required for dynamic programming is to use pruning. Pruning terminates the dynamic programming of a given portion of user habit information against a given treatment model if the partial probability score for that comparison drops below a given threshold. This greatly reduces computation.

Considered to be a generalization of dynamic programming, a hidden Markov model is used in the preferred embodiment to evaluate the probability of occurrence of a sequence of observations O(1), O(2), . . . O(t), . . . , O(T), where each observation O(t) may be either a discrete symbol under the VQ approach or a continuous vector. The sequence of observations may be modeled as a probabilistic function of an underlying Markov chain having state transitions that are not directly observable. In one embodiment, the Markov network is used to model a number of user habits and activities. The transitions between states are represented by a transition matrix A=[a(i,j)]. Each a(i,j) term of the transition matrix is the probability of making a transition to state j given that the model is in state i. The output symbol probability of the model is represented by a set of functions B=[b(j) (O(t)], where the b(j) (O(t) term of the output symbol matrix is the probability of outputting observation O(t), given that the model is in state j. The first state is always constrained to be the initial state for the first time frame of the utterance, as only a prescribed set of left to right state transitions are possible. A predetermined final state is defined from which transitions to other states cannot occur. Transitions are restricted to reentry of a state or entry to one of the next two states. Such transitions are defined in the model as transition probabilities. Although the preferred embodiment restricts the flow graphs to the present state or to the next two states, one skilled in the art can build an HMM model without any transition restrictions, although the sum of all the probabilities of transitioning from any state must still add up to one. In each state of the model, the current feature frame may be identified with one of a set of predefined output symbols or may be labeled probabilistically. In this case, the output symbol probability b(j) O(t) corresponds to the probability assigned by the model that the feature frame symbol is O(t). The model arrangement is a matrix A=[a(i,j)] of transition probabilities and a technique of computing B=b(j) O(t), the feature frame symbol probability in state j. The Markov model is formed for a reference pattern from a plurality of sequences of training patterns and the output symbol probabilities are multivariate Gaussian function probability densities. The patient habit information is processed by a feature extractor. During learning, the resulting feature vector series is processed by a parameter estimator, whose output is provided to the hidden Markov model. The hidden Markov model is used to derive a set of reference pattern templates, each template representative of an identified pattern in a vocabulary set of reference treatment patterns. The Markov model reference templates are next utilized to classify a sequence of observations into one of the reference patterns based on the probability of generating the observations from each Markov model reference pattern template. During recognition, the unknown pattern can then be identified as the reference pattern with the highest probability in the likelihood calculator. The HMM template has a number of states, each having a discrete value. However, because treatment pattern features may have a dynamic pattern in contrast to a single value. The addition of a neural network at the front end of the HMM in an embodiment provides the capability of representing states with dynamic values. The input layer of the neural network comprises input neurons. The outputs of the input layer are distributed to all neurons in the middle layer. Similarly, the outputs of the middle layer are distributed to all output states, which normally would be the output layer of the neuron. However, each output has transition probabilities to itself or to the next outputs, thus forming a modified HMM. Each state of the thus formed HMM is capable of responding to a particular dynamic signal, resulting in a more robust HMM. Alternatively, the neural network can be used alone without resorting to the transition probabilities of the HMM architecture.

A deep feedforward neural network (DNN) is an artificial neural network with multiple hidden layers of units between the input and output layers. Similar to shallow neural networks, DNNs can model complex non-linear relationships. DNN architectures generate compositional models, where extra layers enable composition of features from lower layers, giving a huge learning capacity and thus the potential of modeling complex patterns of muscle movement data. Deep learning does away with hand-crafted feature engineering and to use raw features. The true “raw” features of muscle activities, waveforms, produce excellent larger-scale speech recognition results. A deep learning method called Long short-term memory (LSTM), a recurrent neural network, is normally augmented by recurrent gates called forget gates. Applications use stacks of LSTM RNNs and train them by Connectionist Temporal Classification (CTC) to find an RNN weight matrix that maximizes the probability of the label sequences in a training set, given the corresponding input sequences. CTC achieves both alignment and recognition. CTC-trained LSTM can also be used.

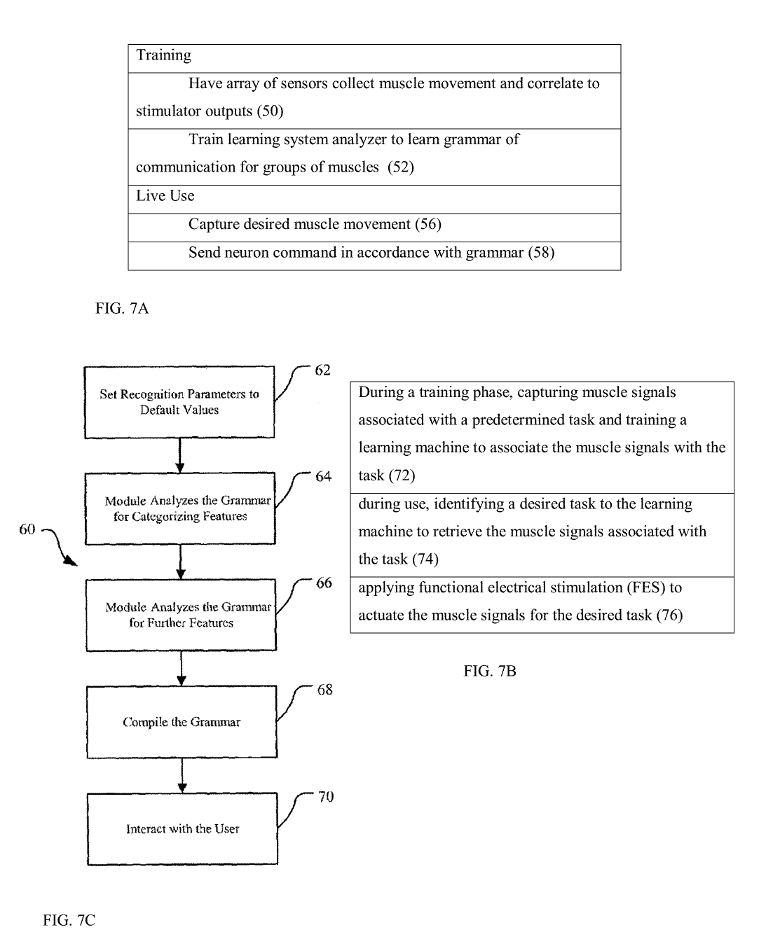

FIG. 7A shows an exemplary process for detecting musculature commands from the brain. This process is similar to speech recognizers, but now is trained on neuron signals rather than speech. The spinal nerves connect to particular muscles or muscle groups which then flex, extend, or rotate, depending on the goal. The nerve signal simultaneously commands a particular muscle action (e.g., flex) while inhibiting the opposite action (e.g., extend). Applying a neuro-recognizer, the system can determine single instructions to the muscles from the brain. In one embodiment, electrodes recorded the electrical activity in the user's brains, body parts such as arms, and hands. A decoder is created that translates the brain activity into muscle movements. The brain can provide pulses of electrical signals forming a grammar for high level instructions of movement, and the system can learn such grammars as detailed below. The process of FIG. 7A has training and deployment phases as follows:

Training

Have array of sensors collect muscle movement and correlate to stimulator outputs (50)

Train learning system analyzer to learn grammar of communication for groups of muscles (52)

Live Use

Capture desired muscle movement (56)

Send neuron command in accordance with grammar (58)

The grammar of sub-muscle movement control is detailed in FIG. 7C.

During the training phase, brain responses are decoded using a plurality of encoding models in order to optimally reconstruct a stimulus. By way of example, a plurality of encoding models can be used. First, a decoding database is created. The decoding database simply consists of a large random sample of stimuli of the same type that will be reconstructed. These stimuli can be obtained from open access sources (e.g., from the internet), they could be tailored to an individual (e.g., from a continuously recording camera or sensors worn by the individual), or they could arise from a generative computational model. For example, if the stimulus to be decoded is a video, the decoding database could be comprised of a large number of different videos. In this embodiment, the decoding database is comprised of different images. Note that for each type of encoding model (i.e. each linearizing feature space), there will be one model for each measurement channel (in this embodiment, for each voxel). Next, each item in the decoding database is processed to determine whether that item will help provide a good reconstruction of the stimulus. For each encoding model, a plurality of processing steps are performed. Each item in the decoding database is first passed through the encoding model(s) estimated for each measurement channel The brain activity predicted by the encoding model is compared to the brain activity actually measured on that measurement channel The predictions for each measurement channel (measure) for each encoding model are then aggregated together. The most likely item in the decoding database is then selected and the top ten, one-hundred, etc. items in the decoding database can be averaged, or a weighted average may be taken. As multiple encoding models are used, their likelihoods can be aggregated together in order to increase the accuracy of reconstruction. The system provides a method for reconstructing completely novel stimuli or mental states, even if they have never been encountered or experienced before by the individual whose brain is being measured. The input can be any number of things, including but not limited to visual stimuli such as images, text and movies; auditory stimuli such as speech; visual or auditory mental imagery such as internal speech; or other cognitive states. In all cases, the stimuli or mental states can be reconstructed by performing decoding using a feature space that reflects how the various stimuli or mental states are encoded. For example, to decode and reconstruct external or internal speech, the feature space could be based on phonemes, syntactic elements or semantic concepts as described below.

FIG. 7B shows an exemplary FES control process. During a training phase, capturing muscle signals associated with a predetermined task and training a learning machine to associate the muscle signals with the task (72). Next, during use, identifying a desired task to the learning machine to retrieve the muscle signals associated with the task (74), and then the system applies functional electrical stimulation (FES) to actuate the muscle signals for the desired task (76).

The system can learn subtleties in controlling sub-muscular movements by learning grammars for the muscle signals, as detailed in FIG. 7C. The muscle signals comprise a plurality of sub-muscle signals to granularly form a movement. The learning machine learns ambulatory muscle control. The learning machine can also learn arm, finger or hand control. When a finger is moved, the system through the learning machine generates an electric impulse that connects to the muscles present in the palm and forearm. This electric signal commands specific muscles to relax and others to flex/tighten. Upon contraction of a muscle, a tendon is pulled. Since tendons are connected to muscles on one end and bones on the other, a pulled tendon causes a bone to move and perform specific actions. Every kind of possible action that fingers perform is controlled/regulated by muscles present in the palm and forehand.

The learning machine learns control of one or more of the following muscle: Trapezius, Levator Scapulae, Major Rhomboids, Minor Rhomboids, Supraspinatus, Infraspinatus, Teres Minor, pronator teres, Gluteus Maximus, Sternocleidomastoid, rectus abdominus, and deltoid. Examples of muscle controls include:

Painting a ceiling—Trapezius

Carrying a heavy bag—Levator Scapulae

Pulling something towards you (like a drawer)—Rhomboids (Major & Minor)

Holding shopping bag away from side of body—Supraspinatus

Brushing back of hair—Infraspinatus & Teres Minor

Turning a doorknob—pronator teres

Rising from sitting/walking upstairs—Gluteus Maximus

Raising head from pillow—Sternocleidomastoid

Initiating getting out of a low chair—rectus abdominus

Raising the arm to wave—deltoid

Similarly, the learning machine learns muscle signals for walking, sitting, standing, or controlling a vehicle. The learning machine can apply FES to sacral nerve stimulation to reduce appetite and thus reduce weight. In addition, the FES can be used to stimulate muscle contraction to build strength and/or reduce weight. As the muscle contracts, the FES achieves exercise of the muscle while the user may be sleeping or working or in door.

The learning machine learns heart nerve stimulation to control blood pressure or to reduce risk of heart failure or heart attack. Heart failure, sometimes known as congestive heart failure, occurs when the heart muscle doesn't pump blood as well as it should. Certain conditions, such as narrowed arteries in the heart (coronary artery disease) or high blood pressure, gradually leave the heart too weak or stiff to fill and pump efficiently. Thus, in case sensors detect Chest pain, Fainting or severe weakness, Rapid or irregular heartbeat associated with shortness of breath, chest pain or fainting, the FES can stimulate heart nerve to increase or decrease pumping action to provide temporary blood support until the patient is seen by the hospital. In another embodiment, if heart rhythm is abnormal, the computer will direct the generator to send electrical pulses to your heart. The pulses travel through the wires to reach the heart. The system can monitor blood temperature, breathing, and other factors. They also can adjust heart rate to changes in your user. The computer also records your heart's electrical activity and heart rhythm.

The learning machine learns sacral nerve stimulation to control bowel movement, bladder movement, or incontinence. In one embodiment to control overactive bladder, the FES sends a sacral nerve stimulation with electrical signals to a sacral nerve through a small device implanted under the user skin. The sacral nerve controls your bladder, sphincter, and pelvic floor muscles. Electrodes may be placed outside the body or in the rectum. For women, the electrodes may be placed in the vagina. The learning machine is trained with the sacral nerve stimulation, and when the user feels the urge to got to the bathroom, the learning machine and FES sends the sacral nerve stimulation to address incontinence issues.

In FIG. 7C, the system can have a module for analyzing a nervous system grammar to determine recognition parameters for a muscle activity recognizer to help improve performance of the recognizer compared to using non-customized recognition parameters. The grammar is first trained by observing neuron firings captured through electronics including embedded brain sensors or EEG sensors on the scalp to detect brain activities responsive to the environment. Such data is used to train the recognizer such as the HMM or neural network or deep learning machine. Once trained, the system can be used to drive the FES controller to generate a similar firing, and the training can be redone to improve the responsiveness of the learned neuron grammar to the environment. The analysis categorizes the neuron grammar according to features of the grammar such as perplexity level and average word length. Depending on the category of the grammar, different recognition parameters are suggested for use in recognizing utterances using the grammar. The confusability level and percentage of numbers in the grammar may be used to set other recognition parameters (e.g., timeout values, skip frames, etc.) although they may not affect the category of the grammar. The suggested recognition parameters are derived through experimentation by testing utterances against grammars using different recognition parameters and evaluating the quality of recognition and the amount of processing power used in the recognition process. This testing also yields thresholds for grammar features (e.g., perplexity, word length, etc.) for use in categorizing grammars. The thresholds may be determined, e.g., by performing experiments with various recognizers attempting to recognize a variety of utterances using different recognition parameters. The results of the recognition attempts can be analyzed to determine relevant characteristics such as the accuracy, speed, and processor load for each attempt. From these analyses, thresholds for categorizing grammars may be determined.

During development, the invention could be used, e.g., to analyze an internal grammar to determine initial recognition parameter settings or to determine settings to supply with a neuro file. For run-time use, the system can analyze a grammar, such as an external neuro muscle grammar, to provide suggestions for recognition settings, although the invention preferably does not alter any settings supplied with the grammar.

Where a supplied parameter applies to a group of multiple recognizer parameters, the system can be used to analyze a grammar and intelligently set individual recognizer parameters within the group based upon the grammar analysis while conforming to the supplied parameter value.

The system is configured to interact with a user to attempt to recognize a muscle actuation from the user and to take appropriate actions. The recognizer is configured to attempt to find the best match of the user's utterances to the neuron models of strings in accordance with applicable grammars. The recognizer is also configured to compile grammars in accordance with recognition parameters to determine the possible muscle movement hypotheses represented as strings to be used in attempting to match with the user's commands. The grammar module is configured to analyze various features of a grammar. These features preferably, but not necessarily, include grammar perplexity, average command length, grammar confusability, and percentage of numbers in the grammar. Grammar perplexity relates to the number of possible strings of acceptable utterances. The perplexity of the grammar is the weighted average number of choices at any point in the sentence.

The module is further configured to use values indicative of the analyzed features to categorize the analyzed grammar. For example, the module can compare the various feature values against corresponding thresholds to determine which of various categories applies to the analyzed grammar. Based on the grammar type, the module is configured to provide recognition parameter suggestions to the recognizer. The same experiments that yielded the thresholds for the grammar types may also yield values for recognition parameters for each of the grammar types. The recognition parameter values, as with the threshold values, will likely vary between different recognizers.

For grammar type 1, with high perplexity and long average word length, the following suggestions are provided: set a pruning value relatively high, limit evaluation of acoustic features on word boundaries (e.g. do not use crossword compilation), use relatively-low word-internal neuron evaluation (e.g. use skip frames), increase timeouts, and use a medium confidence level. For grammar type 2, the suggested recognition parameters are: prune more hypotheses, increase evaluation of acoustic features on word boundaries (e.g. use crossword compilation), use relatively-high word-internal acoustic evaluation (e.g., do not skip frames), increase timeouts, and use a high confidence level. The module is configured to analyze grammars for further features for providing suggestions for other recognition parameters, and possibly overriding the suggestions based upon the grammar type. For example, percentage of grammar words that do not appear in a dictionary may be analyzed. Additionally, the module can determine the percentage of numbers in a grammar, e.g., by looking for digit strings in the grammar. If the percentage of numbers in the grammar is sufficiently high, e.g., exceeds a threshold, or if the grammar contains a high percentage of names (words not found in the dictionary), the fluency of utterances associated with this grammar will likely be low and the module can suggest using a relatively high timeout value for the end of muscle activity. Timeout values include timeouts for the start of muscle activity, end of muscle activity, and maximum allowable muscle activity length.

The module may also suggest turning off skip frames if the module determines that the grammar has a high confusability, thus preserving as many features of the utterances as possible. Grammar confusability relates to how likely it is that different grammar items will be confused by the recognizer for each other. This may be determined by analysis of similarities in phoneme strings, although other techniques are possible.

The grammar analyzed by the module may be internal (part of the system) or external (provided to the system). In the case of an internal grammar, the module may analyze the grammar and provide suggestions for initial recognition parameter settings for future recognition. If the grammar is to be supplied as an external grammar to another system, then the suggested settings may be supplied with the grammar as suggestions for a recognizer to use with the grammar.

In operation, a process 60 for analyzing a grammar and providing recognition parameter settings using the system 10 includes the stages shown. The process 60, however, is exemplary only and not limiting. The process 60 can be altered, e.g., by having stages added, removed, or rearranged (e.g., by having stage 64 discussed below preceded by stage 66 discussed below). At stage 62, recognition parameters are set to default values designed to accommodate a variety of grammars. A table shows initial default recognition parameters. As shown, the recognizer is initially set with a medium confidence level, noise reduction on, pruning off, crossword compilation off, a basic model or models selected, skip frames off, and timeouts normal.

At stage 64, a grammar is analyzed by the module to determine initial recognition parameter suggestions. The grammar may be internal to the system or supplied to the system from outside. The module applies its various algorithms or other techniques for quantizing features of the grammar and compares the determined quantities with thresholds. Based on the comparisons for perplexity and average word length, the module 16 categorizes the grammar into one of four types according to the table. Based on the type of the grammar and the particular recognizer to be used, the module provides initial suggestions for recognition parameters in accordance with the table. Recognition parameters may be calculated based upon parameters associated with the grammar, e.g., a speed vs accuracy or other parameter provided with an external grammar. At stage 66, further grammar features are analyzed and superceding recognition parameter values provided as appropriate. Other features such as confusability and predominance of numbers in the grammar are evaluated by the module and quantities associated with the evaluations are compared to thresholds. Based on these comparisons, overriding recognition parameter value suggestions may be provided. At stage 68, the grammar is compiled. This may occur well in advance of the grammar being used by the recognizer (e.g., during development of a muscle activity application) or relatively close in time to being used by the recognizer (e.g., at run time if the grammar is externally supplied to the system). At stage 70, the system interacts with the user. The suggested recognition parameters from the module are applied by the recognizer. If, however, a recognition parameter was provided with an externally-supplied grammar, the recognizer preferably will not use the corresponding recognition parameter value suggested by the module.

The system includes determining one or more muscle movements responsive to the environment and applying functional electrical stimulation (FES) based on the modeled muscle activities to move muscles responsive to the environment. The grammar is responsive to the needs of the user for responding to the environment. For example, if the environment has a hot region that the person's body contacts, an infrared camera or temperature sensor can be used to detect the danger, then a series of micro muscle movements as modeled using the HMM.

In aspect, a method for rendering virtual or augmented reality content includes capturing images of an instant surrounding within a field of view of an image capturing device; generating virtual images and displaying content to a viewer; and applying functional electrical stimulation (FES) to move muscles responsive to the content.

In another aspect, a method for rendering virtual or augmented reality content uses an array of sensors, the method includes collecting muscle movement and modeling with a learning machine muscle activities as electrical signals from a functional electrical stimulation (FES) stimulator to one or more muscles; capturing images of an environment using an image capturing device; generating virtual images and displaying content to a viewer; determining one or more muscle movements to respond to the environment; and applying functional electrical stimulation (FES) based on the modeled muscle activities to move muscles responsive to the environment.

In yet another aspect, a method for controlling muscle activit first collects muscle electrical signals with an array of sensors, collecting muscle movement and modeling muscle activities as electrical signals from a functional electrical stimulation (FES) stimulator to one or more muscles with a learning machine; determining a desired user motion to respond to an environment and determining one or more muscle movements needed to respond to the environment from the learning machine; and applying functional electrical stimulation (FES) based on the modeled muscle activities to move muscles responsive to the environment.

Implementations of the above embodiments can include one or more of the following. The virtual images of objects are generated by simulating content from the multimedia server based on selection made by the user. For example, video stored in the server can be altered and shown to the user. Virtual content is rendered based on direction of projection of light into the eye of the user, wherein the movement of the eye and the change in eye position at various instants are constantly monitored. As the camera on the eyewear faces the eye, the camera can detect the eye gaze based on the eye image. The virtual content is displayed based on determination of the direction of projection of light into the user's eye. The virtual content is rendered by determining intensity of light to be projected into the user's eyes, such that, the content is clearly visible to the user. The virtual content is displayed based on the intensity of light to be projected into the user's eyes. One or more details in the displayed virtual content are possible to be altered or modified by rendering one or more gesture input received from the user. The outcomes of the gesture input are rendered to the virtual content displayed to the user, to alter or modify one or more details in the virtual content. The virtual content is displayed in one or more formats such as two or three-dimensional formats based on field of view and depth of field of the user's eye and the image capturing device, wherein the field of view and depth of field of the user's eye is detected from the user's eye position. A display surface and a format of display is selected relative to the user's position and orientation.

In another aspect, a method for enabling a user to participate in an activity with one or more other users in virtual or augmented reality includes retrieving information corresponding to the user and the one or more other users; rendering virtual content while applying FES to move muscles responsive to the content; and altering content based on additional information received from the one or more users.

Implementations of the above embodiments can include one or more of the following. The retrieving information from the one or more remote servers includes retrieving information from servers associated with at least one or more social networking platforms. The rendering virtual content includes rendering at least a portion of the virtual content including background scenery depicting a type of activity the user is interested in performing and one or more participants with whom the user is willing to participate in the activity. The type of activity that a user is interested in performing and the participants with whom the user is willing to participate in the activity are determined from one or more among previous activities performed by the user and a set of predefined criteria, which includes preference and interest. The virtual content is rendered based on the user's selection of participants and activity. The displayed virtual content is possible to be altered by the user by providing input corresponding to the type of activity and the participants. The displayed virtual content is altered if the displayed virtual content does not match the theme of the activity or the participants as per the user's criteria. The user is provided an option to select participants to perform the activity with, by broadcasting requests to one or more other users whom the user wishes to participate. The rendering virtual content, wherein the virtual content includes a service provider's web page, wherein the user is allowed to browse through the web page and alter content of the webpage by providing gesture input. Virtual assistance is provided to the user in the form of human irtual assistant, voice assistant or assistance in form of texts, while the user is browsing through the service provider's web page. The system can have a transceiver for remotely receiving signals to provide to the FES and allowing a remote unit to control muscles to perform the desired task. This would allow a person to be remotely “controlled” to perform a task. The remote control can be a human or another machine.

In addition to the FES device, other devices such as pumps can be used with the FES device to affect a condition. For example, the FES in combination with the pneumatic compression apparatus can drive blood through blood vessels in a leg to aid in the prevention of Deep Vein Thrombosis or to improve blood flow in non-leg regions. For the legs, deep and superficial vessels of the foot, calf (and optionally the thigh) are emptied rapidly through a sequential combination of pneumatic compression about the foot and electrical stimulation of the leg muscles. Blood within the veins of the foot is driven out by the application of pneumatic pressure in a bladder substantially coveting the foot. Blood in the veins of the leg is driven out though a squeezing of the gastrocnemius muscle group by direct posterior electrical stimulation thereof. The FES and pump generate a series of rapid pulsatile contractions in succession each of which cause blood to spurt up the deep vein system and out of the calf, while at the same time, the superficial system is emptied into the deep vein system.

The pneumatic foot pump employed enables blood to be substantially driven from the veins of the foot, thus priming the vessels of the leg, particularly the calf Rapid pneumatic compression of the veins of the foot produces an increased peak blood flow velocity in the deep veins and stimulates the production of EDRF. The dynamic timing between the foot compression and calf muscle stimulation will be continuously sensed and generated by the computer such that the blood flow velocity is maximized. In addition, while one foot compression period may last for about 30-50 seconds, the electrical stimulation of the calf muscles utilizes a series of relatively quick stimulating pulses causing a number of muscle contractions to create a number of peaks of high velocity blood flow which again cause increased stimulation of EDRF.

The sensor devices as described herein can be prepared by various methods of depositing, printing, or otherwise including a flexible sensor or flexible sensor circuit on a flexible substrate such as the suit of FIG. 1A. The substrate can include a flexible polymer or inorganic-organic complex, which substrate can be porous in some instance. In other instances, the substrate can be substantially devoid of pores.

Circuits, antennas, and other electrical elements can be constructed on various types of substrates using, for example, laser direct structuring (LDS) and pad printing. LDS uses a laser beam to etch a pattern such as a circuit or antenna pattern into a thermoplastic material that is doped with an organic metal additive. A microscopically rough track is formed where the laser beam hits the doped thermoplastic material. The etched thermoplastic material is then subjected to a copper bath followed by metal plating. In pad printing, a pattern is etched into a plate that is subsequently filled with electrically conductive material. A pad is then placed onto the plate with enough pressure to transfer electrically conductive material to the pad. Finally, the pad is pressed onto a substrate transferring the electrically conductive material to the substrate in the shape of the etched pattern. This process is repeated several times to transfer a sufficient amount of electrically conductive material onto the substrate.

Thermal transferring techniques can be used to make electrically conductive materials. One method includes transferring an electrically conductive material to a substrate by contacting at least a portion of a substrate with electrically conductive material that is disposed on a carrier film. The carrier film may be made of any material that can withstand heat and pressure such that its function with the present methods is retained. For example, the carrier film used with the present methods may withstand heat applied during a hot stamping process such that the carrier film can transfer electrically conductive material to a substrate during a hot stamping process. The carrier film also may be flexible, allowing it to be contacted with substrates of varying dimensions and shapes. Non-limiting examples of suitable carrier films are films produced from polyethylene, polyethylene terephthalate (PET), polypropylene, polyesters, polyimides, polycarbonates, paper, impregnated paper, silicones, fluoropolymers, and copolymers and mixtures thereof. An example of a polyimide film that may be used as the carrier film is sold under the trade-name KAPTON®, which is commercially available from DuPont.

The electrically conductive material may be disposed over at least a portion of the carrier film in a pattern or design that, when adhered to a substrate, can be electrically connected to an electronic device by way of a conductive adhesive, electrically conductive pads, pogo-pins, vias or other methods, thus allowing an electrical current or signal to be transmitted to the electronic device. For instance, the electrically conductive material may be disposed over at least a portion of the carrier film in a pattern that forms a circuit or antenna. The electrically conductive material may be also disposed over at least a portion of the carrier film for the formation of piezo coils, electroluminescent, ground plane, and/or EMI/RFI shielding. When coupled to a pogo-pin, for example, an electrical connection can be made so that an electrical current or signal to be transmitted can be received or transmitted by the device. The electrically conductive material may be disposed, such as in a pattern, using various printing methods. Non-limiting examples of printing methods that can be used to apply the electrically conductive materials to the carrier film include digital printing, flexographic printing, gravure printing, screen printing, and the like.

After exposing the materials to an external source to promote drying, the dried material or materials can be exposed to ambient conditions before additional materials are applied. During this period of time, residual solvent still present after the drying step may continue to dissipate from the material or materials. The electrically conductive material, release coat, dielectric material, adhesive, and/or other decorative and functional materials can be applied to the carrier film to form a layered structure. Accordingly, one embodiment is further directed to a method of making a layered structure comprising: 1) applying a release coat to at least a portion of a carrier film; 2) applying electrically conductive material in a pattern to the carrier film after application of the release coat, wherein the electrically conductive material is applied on top of at least a portion of the release coat; 3) drying the electrically conductive material; 4) applying an adhesive over at least a portion of one or more of the electrically conductive material, release coat, or both; and 5) drying the adhesive. The electrically conductive material and adhesive may be dried after being applied such from 1 to 180 seconds, from 1 to 150 second, 1 to 120 seconds, 1 to 90 seconds, or any of the other drying times previously described. In addition, the layered structure can also include dielectric, decorative and/or functional materials applied over at least a portion of one or more of the release coat, electrically conductive material, adhesive, and carrier film. For example, a dielectric material and/or a decorative material can be applied on top of at least a portion of the release coat and/or the electrically conductive material. The dielectric, decorative, and functional materials may be applied in any desired pattern. The dielectric, decorative and functional materials may be dried independently or together (optionally with the other materials) after being applied, such as from 1 to 180 seconds, from 1 to 150 second, 1 to 120 seconds, 1 to 90 seconds, or any of the other drying times previously described.

The layered structure can be rolled for storage and/or shipping. For example, a layered structure can be formed by separately applying and optionally drying one or more of a release coat, electrically conductive material, adhesive, dielectric material, and decorative material onto a carrier film, and then the layered structure is coiled or recoiled into a roll. Accordingly, it may be desired that at least the outermost surface of the materials applied to the carrier film are tack free. The rolled tack free layered structure can later be unrolled and used in a heat stamping process to transfer electrically conductive materials to a substrate. By “tack free”, it is meant that the layered structure is dried to the touch and adheres to the substrate.

After applying the electrically conductive material (and optionally, other additional materials) onto the carrier film, the carrier film is contacted with a substrate. The substrate can be secured in place to prevent the substrate from moving and then the carrier film is contacted with the substrate. Heat and pressure are then applied to the substrate and carrier film, which includes the electrically conductive material and optionally any of the other materials described herein. For example, a layered structure may be contacted with a substrate that is secured in place or fixtured. Heat and pressure may then be applied to the layered structure and substrate. Heat and pressure can be applied with a hot stamping press, such as a rubber wheel hot stamping press. Heat and pressure are applied such that the electrically conductive material adheres to the substrate. One or more of an adhesive, dielectric material, release coat, and decorative material used with the carrier film can also be adhered to the substrate after applying heat and pressure. For example, an adhesive, dielectric material, and electrically conductive material can be adhered to the substrate after applying heat and pressure.

On embodiment deposits functionalized nanomaterials on flexible substrates. The sensor can be a functionalized nano-material as amperometric biosensor for detecting hydrogen and the change in resistance of the sensor upon contact with hydrogen at room temperature. The resistance change of a semiconducting SWCNT with electrodeposited Pd particles upon exposure to hydrogen. Molecular hydrogen is split on the surface of a Pd particle into atomic hydrogen, which diffuses to the Pd/SWCNT interface. At this interface, a dipole layer is formed, which acts like a microscopic gate electrode that locally changes the charge-carrier concentration The recovery of the room-temperature-operated hydrogen sensor requires a supply of oxygen to remove the hydrogen atoms in the form of water.

Direct electron transfer can be done with various types of CNT electrodes for cytochrome c, horseradish peroxidase, myoglobin, as well as glucose oxidase where the redox-active center is deeply embedded within the protein. In some cases, aligned CNT arrays have been fabricated using self-assembly, followed by the covalent attachment of microperoxidase to the tube ends. A glucose sensor can be obtained by immobilizing glucose oxidase onto SWCNTs, for example. One embodiment includes single-walled carbon nanotubes (SWNT) applied as a coating to the working electrode. The SWNT can be, for example, a mixture of metallic and semiconducting SWNT. The SWNT provide an extremely large surface-to-volume ratio and have useful electrical properties. A sensor according to one embodiment operates by an electrochemical mechanism, whereby the presence of a particular analyte causes electron transfer in the electrochemical system, which can be identified and quantified by measuring a current through the sensor, which can be converted via amperometry to an output voltage. This feature of the present sensor renders it more accurate and reliable than other types of sensors that produce a change in electrical resistance of SWNT in the presence of an analyte. The electrode material can contain or consist of, for example and without limitation, gold, platinum, iridium, silver, silver/silver chloride, copper, aluminum, chromium, or other conductive metals or other conductive materials, or any combination thereof. In one embodiment, the SWNT are functionalized by a coating that includes an enzyme that catalyzes an electron transfer reaction and is specific for the selected analyte, such as glucose. Preferably the reaction is an oxidation reaction. For example, for the detection of glucose as the analyte, the enzyme glucose oxidase (GOx, EC 1.1.3.4) can be used, which specifically catalyzes the oxidation of β-D-glucose to hydrogen peroxide and D-glucono-δ-lactone, which then hydrolyzes to gluconic acid. The enzyme can be a naturally occurring glucose oxidase enzyme which is isolated from a natural source (e.g. cells of Aspergillus niger), or it can be produced recombinantly in transformed or transfected host cells, such as bacterial cells, yeast or fungal cells, or mammalian cells. It can be glycosylated or non-glycosylated. The glucose oxidase enzyme used in the sensor can have a naturally occurring amino acid sequence, or it can have a mutant or engineered amino acid sequence. Different enzyme-functionalized SWNT can be combined in a multiplex sensor that takes advantage of the different sensitivities of each enzyme and their different resistance to inhibition induced by potentially interfering substances that might be encountered in a saliva sample. The sensor detects levels of glucose in saliva or another fluid by keeping track of the electrons passed through the glucose oxidase enzyme coated on the working electrode and measuring the resulting current, which is detected by an amperometry detection circuit and expressed as a change in output voltage. The sensing performance can be further improved by modifying the enzyme-coated electrode with various materials, including biomolecular or porous films or membranes. Such materials include, but are not limited to, carbon nanotubes, graphite, nanowires, gold nanoparticles (GNp), Pt nanoparticles, chitosan, bovine serum albumin (BSA), and Prussian Blue or other materials with similar properties. In one embodiment, the sensor of one embodiment detects glucose via an electrical signal resulting from the glucose oxidase reaction performed on functionalized SWNT connected to a detection circuit. It does not require any additional chemical reactions (e.g. peroxidase reaction) or optical detection means to detect the reaction products.

The system can be implemented on a flexible substrate with microneedles formed by impressing a bed of nails template onto the flexible substrate, onto which sweat can be captured and glucose and other important analytes can be captured. The system can be designed for single use (i.e., disposable) or for repeated use, with rinsing off, washing, or simple displacement of the sweat sample between readings. It can be used for real-time, noninvasive glucose monitoring for individuals at home and around clock. Through continuous or periodic glucose and/or analyte monitoring, additional temporal information can be obtained, such as trends, magnitude, duration, and frequency of certain glucose/analyte levels; this would allow tracking of data for better and more accurate assessment of a disease as well as the overall health condition of an individual. For example, the sensor system can activate an alarm for unusual or extreme glucose/analyte levels, decreasing the nursing workload when trying to maintain tight glycemic control. Such a system can also facilitate automatic feedback-controlled insulin delivery in an insulin delivery system, such as an artificial pancreas or insulin pump.

The flexible electronics can incorporate microneedles to extract deep subdermal fluids and/or to inject chemicals such as drugs into the blood stream upon detection of a trigger. For example, for diabetes, some microneedles extract sweats and/or glands secretion of glucose, and the glucose level is determined, and in a closed loop, drugs can be injected via another set of microneedles and suitable valves or seals that are opened on command. One such seal is opened by heaters on the microneedles to release the drugs. In one embodiment, a flexible skin patch can be made with functionalized macromolecules such as CNTs as sensors that detect humidity, glucose, pH, and temperature. The glucose sensor takes into account pH and temperature to improve the accuracy of the glucose measurements taken from sweat. If the skin patch senses high glucose levels, heaters trigger microneedles to dissolve a coating and release the drug metformin just below the skin surface. FIG. 5A shows an exemplary flexible printed electronic with microneedles theron. The microneedles form an interface with the skin for detecting analyte or sugar levels in the person. In certain embodiments, a portion of the needles can inject medication in response to the detected levels in the person to form a close loop control system.

One embodiment provides a large skin patch with a sweat collection region to collect low quantity body fluid such as sweat. A flexbile electronic pad can be printed for sweat collection with a channel layer, a container layer, and a vent layer. In some variations, the layers may be combined into a single layer and/or other layers may be added. The channel layer of the fixed volume device may contact the skin surface and direct sweat from the skin surface to an opening. On the skin surface, the sweat may be within or excreted from one or more sweat pores in contact with, or adjacent to, the channel layer. Typically, the container layer may be in fluid communication with an opening in the channel layer and may be in contact with the vent layer. The vent layer may be in contact with the container layer and may allow air to escape during sweat collection. The channel layer may have any number of channels to contact the skin for sweat collection. Upon contacting the skin surface, the channel layer may deform to contact as much skin as possible so that the channels may efficiently route sweat to the opening. The channel layer may have any suitable geometry or have any suitable dimensions. For example, the channel layer may have a thickness of about two hundred micrometers and the opening may have a diameter of less than about seven hundred micrometers. In some embodiments, the opening may have a diameter of greater than three hundred micrometers. The top side of the channel layer may define a bottom side of the container for holding the collected sweat. In these instances, the channel layer may or may not include one or more electrodes in contact with the container that is positioned to contact sweat within the container.

The container layer may be positioned on top of or extend from the channel layer, and may have the same size and shape as the channel layer or be of a different size and/or shape. The channel layer may include at least one opening opposite the container layer to draw the sweat from the skin surface. The container layer may include a feature that defines at least one side of the container. The feature may be a hole, a well, an indentation, an absorbent portion, or the like. The thickness of the container layer may be selected based on one or more factors such as the shape of the container, the volume of the container, or rigidity required for the container to maintain its shape when the channel layer is deformed. For example, the container layer may have a thickness of approximately 100, 200, 500, 700, or 1,000 micrometers. Like the channel layer, the container layer may also comprise one or more electrodes positioned to contact sweat within the container. The electrodes may be used in conjunction with a measurement device to, for example, determine when the container contains the fixed volume of sweat and/or to measure the sweat glucose level. The vent layer may be positioned on top of or extend from the container layer. In some variations, the functions performed by the vent layer may be performed by the container layer. The vent layer may reduce evaporation of sweat and/or provide an escape route for air within the container. In general, larger vents provide more fluid flow because the air can escape quickly but may allow more sweat to evaporate from the container.

To measure a glucose level from sweat, a system includes collecting a predetermined volume of sweat from skin using a skin patch and measuring the amount of glucose within the volume of sweat. The skin patch may be attached to any location on the body covered by skin. Typically, however, the skin patch is placed on a fingertip, hand, or forearm as these areas have a higher density of sweat glands, are easily accessible, and are currently used by diabetic patients for blood glucose testing. The skin patch may be a skin patch as described above or may be another skin patch that is configured to collect a predetermined volume of sweat. The predetermined volume of sweat may be less than about one-quarter microliter of sweat, about one-half microliter of sweat, about one microliter of sweat, about two microliters of sweat, about five microliters of sweat, about ten microliters of sweat, or any other suitable volume. Measuring the amount of glucose may comprise contacting the skin patch with a measurement device.

In some embodiments, the method also includes stimulating sweat production. Sweat production may be simulated chemically, e.g., by delivering pilocarpine to the skin surface. The pilocarpine may be wiped onto the skin surface prior to attachment of the skin patch. Sweat may also be stimulated by delivering heat or one or more other forms of energy to the surface of the skin. The patch itself may comprise a physical, chemical, or mechanical mechanism of inducing a local sweat response. For example, the patch may comprise pilocarpine, alone or with a permeation enhancer, or may be configured for iontophoretic delivery. Similarly, the patch may comprise one or more chemicals capable of inducing a local temperature increase, thereby initiating a local sweat response. In a like manner, the patch may also comprise one or more heaters for sufficient localized heating of the skin surface to induce an enhanced local sweat response.

The microneedles are formed above a substrate with a plurality of microneedle base parts projected from the substrate integrally. Then a microneedle tip part is formed on the top of each of the plurality of microneedle base parts, with in vivo solubility and biodegradability. A microneedle tip part intrusion recess is formed in the microneedle base part; and the microneedle tip part partially intrudes into the microneedle tip part intrusion recess. The plurality of microneedles is punctured into the skin so that the microneedle tip parts remain under the skin. The tip parts can administer an objective substance such as medication. The administration volume of the objective substance by the microneedle array as part of the flexible substrate 1 is controlled by the processor and varies depending on the EKG, heart rate, glucose level, K/Na level as detected by the electronics, and further based on the seriousness of symptom, the age, gender and weight of the administration subject, the administration period and intervals, and the type of active ingredients, and it is possible to select from the range that the administration volume as the medical active ingredients reaches the effective dose. Moreover, it is also possible to administrate the objective substance by the microneedle array on the flexible substrate 1, once a day, or divisionally twice or three times a day.

The applicable objective substances on the tip parts can include, as for hormones, luteinizing hormone-releasing hormone analog, insulin, faster-acting insulin analog, long-acting insulin analog, ultra-long-acting insulin analog, growth hormone, PEGylation human growth hormone analog, somatomedin C, natriuretic peptide, glucagon, follicle-stimulating hormone, GLP-1 analog, parathyroid hormone analog, and as for enzymes, t-PA, glucocerebrosidase, alpha-galactosidase A, alpha-L-iduronidase, acid alpha-glucosidase, iduronate-2-sulfatase, human N-acetylgalactosamine-4-sulfatase, urate oxidase, deoxyribonuclease, and as for blood coagulation/fibrinolysis-associated factors, blood coagulation factor VIII, blood coagulation factor VII, blood coagulation factor IX, thrombomodulin, and as for serum proteins, albumin, and as for interferons, interferon-alpha, interferon-beta, interferon-gamma, PEGylation interferon-alpha, and as for erythropoietins, erythropoietin, erythropoietin analog, PEGylation erythropoietin, and as for cytokines, G-CSF, G-CSF derivative, interleukin-2, bFGF, and as for antibodies, mouse anti-CD3 antibody, humanized anti-EGF receptor antibody, chimeric anti-CD20 antibody, humanized anti-RS virus antibody, chimeric anti-TNF-alpha antibody, chimeric anti-CD25 antibody, humanized anti-IL6 receptor antibody, calicheamicin binding humanized anti-CD33 antibody, humanized anti-VEGF antibody, MX-DTPA binding mouse anti-CD20 antibody, human anti-TNF-alpha antibody, chimeric anti-EGFR antibody, humanized anti-VEGF antibody fragment, humanized IgE antibody, human anti-complement-C5 antibody, human anti-EGFR antibody, human anti-IL12/IL23-p40 antibody, human anti-IL-1-beta antibody, human anti-RANKL antibody, humanized anti-CCR4 antibody, PEGylation humanized anti-TNF-alpha antibody Fab, and as for fusion proteins, soluble TNF receptor Fc fusion protein, CTLA4-modified Fc fusion protein, Fc-TPOR agonist peptide fusion protein, VEGFR-Fc fusion protein, and as for vaccines, tetanus toxoid, diphtheria toxoid, pertussis vaccine, inactivated polio vaccine, live polio vaccine, diphtheria-tetanus combined toxoid, pertussis diphtheria tetanus mixed vaccine, haemophilus influenzae b (Hib) vaccine, hepatitis B vaccine, hepatitis A vaccine, influenza hemagglutinin vaccine, rabies vaccine, Japanese encephalitis vaccine, Weil's disease autumnalis combined vaccine, pneumococcus vaccine, human papilloma virus vaccine, mumps vaccine, varicella vaccine, rubella vaccine, measles vaccine, rotavirus vaccine, norovirus vaccine, RSV vaccine, BCG vaccine. Further, any substances having an effect of assisting activation of the medical agents or an effect of immune system adjustment, are also included in the medical agents of one embodiment, and for example, any adjuvants commonly used for manufacturing of vaccine formulations can be used. As for adjuvants, hardly water-soluble adjuvant, hydrophilic gel adjuvant or water-soluble adjuvant can be used. As for hardly water-soluble adjuvants, for example, retinoid such as retinoic acid, imiquimod, and imidazoquinolines such as Resquimod (R-848), 4-amino-α,α, 2-dimethyl-1H-imidazo[4,5-c]quinoline-1-ethanol (R-842 (made by 3M Pharmaceuticals, etc.); Journal of Leukocyte Biology (1995) 58: see 365-372), 4-amino-α,α, 2-trimethyl-1H-imidazo[4,5-c]quinoline-1-ethanol (S-27609 (made by 3M Pharmaceuticals, etc.); Journal of Leukocyte Biology (1995) 58: see 365-372), 4-amino-2-ethoxymethyl-α, α-dimethyl-1H-imidazo[4,5-c]quinoline-1-ethanol (S-28463 (made by 3M Pharmaceuticals, etc.); Antivirul Research (1995) 28: see 253-264), and Loxoribine, Bropirimine, oleic acid, liquid paraffin, and Freund's adjuvant are included. As for hydrophilic gel adjuvants, for example, aluminum hydroxide and aluminum phosphate are included. As for water-soluble adjuvants, for example, alpha-defensin, beta-defensin, cathelicidin, sodium alginate, poly[di(carboxylatophenoxy)phosphazene], Quil A, polyethylene imine are included. The preferable adjuvants are hydrophilic gel adjuvants and water-soluble adjuvants. As for hydrophilic gel adjuvants, aluminum hydroxide and aluminum phosphate are included.

In one embodiment, a system for manufacturing a flexible sensor device can include any combination of the thermal transfer printing system, plasma jet sprayer, inkjet printer, compositions, and/or other features described herein for inkjet printing onto a flexible substrate in order to prepare a flexible sensor device.

Methods such as physical vapor deposition, magnetron sputtering, plasma-enhanced chemical vapor deposition, hotolithography, and chemical vapor deposition may not be suitable for materials that cannot be processed under high vacuum due to outgassing issues. Screen printing is an inexpensive process for planar substrates.

Spin coating, blade coating and spray coating can be used. Spin coating is a method of coating which is widely used within lab scale OPV manufacturing and in general within the semiconductor industry, to dispense liquids in very uniform layers on planar substrates. In one embodiment, a Laurell lab scale spin coater can be used, where the substrate is mounted on a chuck that rotates the sample while dispensing the liquid onto the sample, first distributing the liquid and secondly applying a high rotational velocity to dispersing the liquid into a uniform film thickness. Slot-die coating is a non-contact large-area processing method for the deposition of homogeneous wet films with high cross-directional uniformity. The slot-die coating head is made from stainless steel and contains an ink distribution chamber, feed slot, and an up- and downstream lip. An internal mask (shim) defines the feed slot width and allows stripe coating.

Inkjet and aerosol printing can be used but may need post-deposition thermal treatment for the formation of a uniform film and removing organic contaminants. Spray coating is widely known as an (industrial) method for car body painting and from graffiti artists using spray cans. The functional fluid or ink is atomized at the nozzle of the spray head, which generates a continuous flow of droplets. Pneumatic-based systems use a stream of pressurized air or gas (e.g. helium, nitrogen or argon) that breaks up the liquid into droplets at the nozzle. Parameters for the atomization process are surface tension, viscosity, fluid density, gas flow properties, and nozzle design. The quality of the coated layer is defined by the wetting behavior, surface properties, working distance, coating speed, droplet sizes, and the amount of sprayed layers. Besides the fluid-surface interaction the kinetic impact of the droplets influence the spreading of the droplets. An airbrush gun can be used, but other spray generation methods can be used such as ultrasonication with directed carrier gases, or electro-spraying.

In one embodiment, a method of manufacturing a flexible sensor device can include plasma spraying (plasma jetting or simply jetting) a nanosensor-containing composition onto a flexible substrate so as to deposit and retain one or more of nanosensors in a first predetermined pattern of a first macrosensor on the flexible substrate. The flexible substrate that has jet-printed nanosensors can be configured to have a desired degree of elongation, contraction and distortion while retaining sensing functions of the nanosensors. Such configuration can be achieved by the flexible substrate having such flexibility. Also, the jetted composition can include components, such as binders, elastomers, polymers, or the like, that provide post printing flexibility. In another embodiment, the method of manufacture can include jetting a second nanosensor-containing composition onto the flexible substrate. The second nanosensor-containing composition can include nanosensors that are different from the other nanosensors. The nanosensors can be configured to detect different target substances. Alternatively, the nanosensors can be a different type that detects the same target substance. In yet another embodiment, manufacturing can include jetting a conducting polymer-containing composition onto the flexible substrate so as to form a sensor circuit that is operably coupled with at least one jet printed nanosensor. The sensor circuit can include circuit components formed from the conducting polymer. The jetting of the conducting polymer-containing composition can also include the jetting of components that form a conducting polymer, such as, monomers, polymerizers, dopants, reactants, binders, polymers, conductive components, metallic components, and the like that can form a conducting polymer in a circuit configuration. Thus, the printing of a conducting polymer can be performed by printing components that combine to form a conducting polymer on the substrate. In one embodiment, manufacturing can include jetting a nanowire complex-containing composition onto the flexible substrate so as to form a sensor circuit that is operably coupled with at least one jet printed nanosensor. The sensor circuit can include circuit components formed from the nanowire. The jetting of the nanowire-containing composition can also include the jetting of components that form a nanowire, such as, semiconductor materials, monomers, polymerizers, dopants, reactants, binders, polymers, and the like that can form a nanowire in a circuit configuration. Thus, the printing of a nanowire polymer can be performed by printing components that combine to form a conducting polymer on the substrate.

An atmospheric-pressure plasma jet deposition can be done using a dielectric barrier discharge and can provide high-throughput processing and can coat three-dimensional objects. The presence of a dielectric material between the electrodes at the nozzle reduces the current filament, resulting in lowtemperature deposition suitable for low glass transition temperature materials.

The plasma jet printer consists of a quartz nozzle containing two copper electrodes and connected to a high-voltage (1 to 15 kV AC) power supply. A fixed aerosol flow is provided with plasma turned-off. A dielectric barrier discharge of helium is generated upon applying a potential between the electrodes. A container with a colloid of the functionalized nanomaterial to be deposited is placed on a nebulizer that generates an aerosol of the colloid, and the aerosol is carried by a helium carrier gas into the quartz tube containing the plasma. The deposition takes place at room temperature on the substrate placed closely to the nozzle. The sprayer jet does not need a vacuum pump and vacuum chamber as the process takes place at atmospheric pressure to reduce damage to the functionalized multiwalled carbon nanotubes.

In one embodiment, manufacturing can include plasmajetting a conducting polymer-containing composition and a nanowire complex-containing composition onto the flexible substrate so as to form a sensor circuit that is operably coupled with at least one jetted nanosensor. The conducting polymer and nanowire complex can cooperate to form the sensor circuit. The conducting polymer-containing composition can be retained in a separate reservoir from the nanowire complex-containing composition. As before, the formation of the sensor circuit can be performed by printing pre-conducting polymer components and/or pre-nanowire components that form conducting polymers and/or nanowires on the substrate so as to form the sensor circuit.

In one embodiment, the flexible substrate can be incorporated into a wearable garment. Wearable garments that include sensors can be used for sensing biometric data as well as sensing target substances as described herein. In some instances, the biometric data can be obtained from detecting target substances. As such, the method of manufacture can include configuring the flexible substrate having the jet-printed nanosensors with sufficient flexibility for being a component of a wearable garment such that the macrosensor is capable of sensing biometric data of a subject wearing the wearable garment. The sensors can detect a chemical that is provided from a subject wearing the garment, and the detection of the chemical or determination of the amount or concentration of the chemical in or on the subject can provide biometric data. Biometric data can then be used for health purposes and/or determine the health state of the subject.

In one embodiment, a nanosensor-containing composition can be jetted onto the flexible substrate so as to deposit and retain one or more of nanosensors in at least a second predetermined pattern of at least a second macrosensor on the flexible substrate. The first and second macrosensors can be separated by cutting the flexible substrate. Alternatively, the first macrosensor can be placed onto the second macrosensor and the flexible substrate can be adhered together to form a pouch having both macrosensors. Also, this can include operably coupling a second macrosensor with the first macrosensor.

The method of manufacture can include placing a second flexible substrate onto the flexible substrate having the jet-printed nanosensors, and bonding the second flexible substrate to the flexible substrate having the jet-printed nanosensors. This can be used to prepare the sensor devices as described herein. Also, the flexible substrate can be folded onto itself and bonded to form a container as described herein.

Accordingly, a method of preparing a flexible sensor device by jet printing can include jetting a sensor-containing composition onto a flexible substrate so as to deposit and retain one or more sensors in a first predetermined pattern of a first sensor (e.g., macrosensor) on the flexible substrate. The jet printed sensor can have the flexibility, elongation, contraction, and/or distortion properties as described herein. The flexible substrate having the jet-printed sensors is configured to have a desired degree of elongation, contraction, and distortion while retaining sensing functions of the sensors. Also, the jetted composition can include components, such as binders, elastomers, polymers, or the like, that provide post printing flexibility.

In one embodiment, the method of manufacturing a flexible sensor device can also include any one or combination of the following: jetting a second sensor-containing composition onto the flexible substrate; jetting a conducting polymer-containing composition onto the flexible substrate so as to form a sensor circuit that is operably coupled with at least one jet printed sensor; jetting a nanowire complex-containing composition onto the flexible substrate so as to form a sensor circuit that is operably coupled with at least one jet printed sensor; jetting a nanowire complex-containing composition onto the flexible substrate so as to form a sensor circuit that is operably coupled with at least one jetted sensor and the jetted nanowire complex containing sensor circuit, wherein the conducting polymer-containing composition is retained from a separate reservoir from the nanowire complex-containing composition; or jetting a sensor-containing composition onto the flexible substrate so as to deposit and retain a plurality of sensors in at least a second predetermined pattern of at least a second macrosensor on the flexible substrate; or operably coupling a second macrosensor with the first sensor (e.g., first macrosensor). Such manufacturing steps can be performed as described herein or known in the art. The printed sensors can be individual sensor or any number of sensors together so as to form a macrosensor. Macrosensors are considered to be a sensor formed of sensors and/or nanosensors.

In one embodiment, a method of manufacturing a flexible sensor device having one or more sensor circuits by jet printing. The jet printing method can include jetting at least one composition having components for forming a sensor circuit onto a flexible substrate so as to form and retain at least one sensor circuit on the flexible substrate in a predetermined pattern. The sensor circuit can be configured for sensing an interaction with a target substance. The flexible substrate having the jet-printed sensor circuit can be configured to have a desired degree of elongation, contraction, and distortion while retaining sensing functions of the sensor circuit.

In one embodiment, the method of manufacture can also include any of the following: preparing the at least one composition having components for forming the sensor circuit to have a conducting polymer-containing composition configured for being jetted onto the flexible substrate; preparing the at least one composition having components for forming the sensor circuit to include a nanowire complex-containing composition configured for being jetted onto the flexible substrate; jetting a conducting polymer-containing composition onto the flexible substrate so as to form the sensor circuit; jetting a nanowire complex-containing composition onto the flexible substrate so as to form the sensor circuit; jetting a conducting polymer-containing composition and a nanowire complex-containing composition onto the flexible substrate so as to form the sensor circuit; jetting a nanosensor-containing composition onto the flexible substrate so as to deposit and retain a plurality of nanosensors in a first predetermined pattern of a first macrosensor on the flexible substrate, said flexible substrate having the jet-printed nanosensors being configured to have a desired degree of elongation, contraction and distortion while retaining sensing functions of the nanosensors, the first macrosensor being operably coupled with the at least one sensing circuit and being configured to interact with a target substance; or configuring the flexible substrate having the jet-printed nanosensors with sufficient flexibility for being a component of a wearable garment such that the macrosensor is capable of sensing biometric data of a subject wearing the wearable garment. Also, the method can include placing a second flexible substrate onto the flexible substrate having the jet-printed sensor circuit, and bonding the second flexible substrate to the flexible substrate having the jet-printed sensor circuit. Such manufacturing steps can be performed as described herein or known in the art.

A chain of wells and channels on substrates can be formed as microfluidic cassettes or devices that can be used to effect a number of manipulations on a sample to ultimately result in target analyte detection or quantification. These manipulations can include cell handling (cell concentration, cell lysis, cell removal, cell separation, etc.), separation of the desired target analyte from other sample components, chemical or enzymatic reactions on the target analyte, detection of the target analyte, etc. The devices can include one or more wells for sample manipulation, waste or reagents; channels to and between these wells, including channels containing electrophoretic separation matrices; valves to control fluid movement; on-chip pumps such as electroosmotic, electrohydrodynamic, or electrokinetic pumps; and detection systems comprising electrodes, as is more fully described below. The devices of can be configured to manipulate one or multiple samples or analytes.

The microfluidic devices are used to detect target analytes in samples. By “target analyte” or “analyte” or grammatical equivalents herein is meant any molecule, compound or particle to be detected. As outlined below, target analytes preferably bind to binding ligands, as is more fully described above. As will be appreciated by those in the art, a large number of analytes may be detected using the present methods; basically, any target analyte for which a binding ligand, described herein, may be made may be detected using the methods of the invention.

Suitable analytes include organic and inorganic molecules, including biomolecules. In one embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (TM) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This is particularly advantageous in the systems of the system, as a reduced salt hybridization solution has a lower Faradaic current than a physiological salt solution (in the range of 150 mM).

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as nucleosides.

In one embodiment, the system provides methods of detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 basepairs, with fragments of roughly 500 basepairs being preferred in some embodiments. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

The probes (including primers) are made to hybridize to target sequences to determine the presence or absence of the target sequence in a sample. Generally speaking, this term will be understood by those skilled in the art.

The target sequence may also be comprised of different target domains; for example, in “sandwich” type assays as outlined below, a first target domain of the sample target sequence may hybridize to a capture probe or a portion of capture extender probe, a second target domain may hybridize to a portion of an amplifier probe, a label probe, or a different capture or capture extender probe, etc. In addition, the target domains may be adjacent (i.e. contiguous) or separated. For example, when ligation chain reaction (LCR) techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below.

In one embodiment, the target analyte is a protein. As will be appreciated by those in the art, there are a large number of possible proteinaceous target analytes that may be detected using the system. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In one embodiment, the amino acids are in the (S) or L-configuration. As discussed below, when the protein is used as a binding ligand, it may be desirable to utilize protein analogs to retard degradation by sample contaminants.

Suitable protein target analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g. respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus); hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (PA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-a and TGF-(3), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (4) other proteins (including a-fetoprotein, carcinoembryonic antigen CEA, cancer markers, etc.).

In addition, any of the biomolecules for which antibodies may be detected may be detected directly as well; that is, detection of virus or bacterial cells, therapeutic and abused drugs, etc., may be done directly.

Suitable target analytes include carbohydrates, including but not limited to, markers for breast cancer (CA 15-3, CA 549, CA 27.29), mucin-like carcinoma associated antigen (MCA), ovarian cancer (CA 125), pancreatic cancer (DE-PAN-2), prostate cancer (PSA), CEA, and colorectal and pancreatic cancer (CA 19, CA 50, CA 242).

Suitable target analytes include metal ions, particularly heavy and/or toxic metals, including but not limited to, aluminum, arsenic, cadmium, selenium, cobalt, copper, chromium, lead, silver and nickel.

These target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc.

At least one channel or flow channel allows the flow of sample from the sample inlet port to the other components or modules of the system. The collection of channels and wells is sometimes referred to in the art as a “mesoscale flow system”. The flow channels may be configured in a wide variety of ways, depending on the use of the channel For example, a single flow channel starting at the sample inlet port may be separated into a variety of smaller channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used.

In addition to the flow channel system, the microfluidic devices are configured to include one or more of a variety of components, herein referred to as “modules”, that will be present on any given device depending on its use. These modules include, but are not limited to: sample inlet ports; sample introduction or collection modules; cell handling modules (for example, for cell lysis, cell removal, cell concentration, cell separation or capture, cell growth, etc.); separation modules, for example, for electrophoresis, dielectrophoresis, gel filtration, ion exchange/affinity chromatography (capture and release) etc.; reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), and nucleic acid sequence based amplification (NASBA)), chemical, physical or enzymatic cleavage or alteration of the target analyte, or chemical modification of the target; fluid pumps; fluid valves; thermal modules for heating and cooling; storage modules for assay reagents; mixing chambers; and detection modules.

In one embodiment, the devices include a cell handling module. This is of particular use when the sample comprises cells that either contain the target analyte or that must be removed in order to detect the target analyte. Thus, for example, the detection of particular antibodies in blood can require the removal of the blood cells for efficient analysis, or the cells (and/or nucleus) must be lysed prior to detection. In this context, “cells” include eukaryotic and prokaryotic cells, and viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, cell concentration modules, and cell separation or capture modules. In addition, as for all the modules of the invention, the cell handling module is in fluid communication via a flow channel with at least one other module of the invention.

In one embodiment, the cell handling module includes a cell lysis module. The cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In one embodiment, the cell lysis module comprises a cell lysing agent, such as guanidium chloride, chaotropic salts, enzymes such as lysozymes, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample. The cell lysis module may also include, either internally or externally, a filtering module for the removal of cellular debris as needed. This filter may be microfabricated between the cell lysis module and the subsequent module to enable the removal of the lysed cell membrane and other cellular debris components.

In one embodiment, the cell handling module includes a cell separation or capture module. This embodiment utilizes a cell capture region comprising binding sites capable of reversibly binding a cell surface molecule to enable the selective isolation (or removal) of a particular type of cell from the sample population, for example, white blood cells for the analysis of chromosomal nucleic acid, or subsets of white blood cells. These binding moieties may be immobilized either on the surface of the module or on a particle trapped within the module (i.e. a bead) by physical absorption or by covalent attachment. Suitable binding moieties will depend on the cell type to be isolated or removed, and generally includes antibodies and other binding ligands, such as ligands for cell surface receptors, etc.

Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

Alternatively, a cellular “sieve” can be used to separate cells on the basis of size. This can be done in a variety of ways, including protrusions from the surface that allow size exclusion, a series of narrowing channels, a weir, or a diafiltration type setup.

In one embodiment, the cell handling module includes a cell removal module. This may be used when the sample contains cells that are not required in the assay or are undesirable. Generally, cell removal will be done on the basis of size exclusion as for “sieving”, above, with channels exiting the cell handling module that are too small for the cells.

In one embodiment, the cell handling module includes a cell concentration module. As will be appreciated by those in the art, this is done using “sieving” methods, for example to concentrate the cells from a large volume of sample fluid prior to lysis.

In one embodiment, the devices include a separation module. Separation in this context means that at least one component of the sample is separated from other components of the sample. This can comprise the separation or isolation of the target analyte, or the removal of contaminants that interfere with the analysis of the target analyte, depending on the assay.

In one embodiment, the separation module includes an electrophoresis module where molecules are primarily separated by different electrophoretic mobilities caused by their different molecular size, shape and/or charge. Microcapillary tubes are used in microcapillary gel electrophoresis (high performance capillary electrophoresis (HPCE)). One advantage of HPCE is that the heat resulting from the applied electric field is efficiently disappated due to the high surface area, thus allowing fast separation. The electrophoresis module serves to separate sample components by the application of an electric field, with the movement of the sample components being due either to their charge or, depending on the surface chemistry of the channel, bulk fluid flow as a result of electroosmotic flow (EOF).

As will be appreciated by those in the art, the electrophoresis module can take on a variety of forms, and generally comprises an electrophoretic channel and associated electrodes to apply an electric field to the electrophoretic channel. Waste fluid outlets and reservoirs are present as required. Electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose.

In one embodiment, the devices include a reaction module. This can include physical, chemical or biological alteration of one or more sample components. Alternatively, it may include a reaction module wherein the target analyte alters a second moiety that can then be detected; for example, if the target analyte is an enzyme, the reaction chamber may comprise an enzyme substrate that upon modification by the target analyte, can then be detected. In this embodiment, the reaction module may contain the necessary reagents, or they may be stored in a storage module and pumped as outlined herein to the reaction module as needed. In one embodiment, the reaction module includes a chamber for the chemical modification of all or part of the sample. For example, chemical cleavage of sample components (CNBr cleavage of proteins, etc.) or chemical cross-linking can be done. In one embodiment, the reaction module includes a chamber for the biological alteration of all or part of the sample. For example, enzymatic processes including nucleic acid amplification, hydrolysis of sample components or the hydrolysis of substrates by a target enzyme, the addition or removal of detectable labels, the addition or removal of phosphate groups, etc.

In one embodiment, the target analyte is a nucleic acid and the biological reaction chamber allows amplification of the target nucleic acid. Suitable amplification techniques include, both target amplification and probe amplification, including, but not limited to, polymerase chain reaction (PCR), ligase chain reaction (LCR), strand displacement amplification (SDA), self-sustained sequence replication (3SR), QB replicase amplification (QBR), repair chain reaction (RCR), cycling probe technology or reaction (CPT or CPR), and nucleic acid sequence based amplification (NASBA). In most cases, double stranded target nucleic acids are denatured to render them single stranded so as to permit hybridization of the primers and other probes of the invention. One embodiment utilizes a thermal step, generally by raising the temperature of the reaction to about 95° C., although pH changes and other techniques such as the use of extra probes or nucleic acid binding proteins may also be used. A probe nucleic acid (also referred to herein as a primer nucleic acid) is then contacted to the target sequence to form a hybridization complex. By “primer nucleic acid” herein is meant a probe nucleic acid that will hybridize to some portion, i.e. a domain, of the target sequence. Probes of the system are designed to be complementary to a target sequence (either the target sequence of the sample or to other probe sequences, as is described below), such that hybridization of the target sequence and the probes of the system occurs. As outlined below, this complementarity need not be perfect; there may be any number of base pair mismatches which will interfere with hybridization between the target sequence and the single stranded nucleic acids of the system. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

Once the hybridization complex between the primer and the target sequence has been formed, an enzyme, sometimes termed an “amplification enzyme”, is used to modify the primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides are added to the primer to extend its length. Once the enzyme has modified the primer to form a modified primer, the hybridization complex is disassociated. After a suitable time or amplification, the modified primer is moved to a detection module and incorporated into an assay complex, as is more fully outlined below. The assay complex is covalently attached to an electrode, and comprises at least one electron transfer moiety (ETM), described below. Electron transfer between the ETM and the electrode is then detected to indicate the presence or absence of the original target sequence, as described below.

In one embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA).

In one embodiment, the target amplification technique is PCR. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostabile polymerase such as Taq I polymerase and thermal cycling.

In one embodiment, the target amplification technique is Strand displacement amplification (SDA) where a single stranded target nucleic acid, usually a DNA target sequence, is contacted with an SDA primer. An “SDA primer” generally has a length of 25-100 nucleotides, with SDA primers of approximately 35 nucleotides being preferred. An SDA primer is substantially complementary to a region at the 3′ end of the target sequence, and the primer has a sequence at its 5′ end (outside of the region that is complementary to the target) that is a recognition sequence for a restriction endonuclease, sometimes referred to herein as a “nicking enzyme” or a “nicking endonuclease”, as outlined below. The SDA primer then hybridizes to the target sequence. The SDA reaction mixture also contains a polymerase (an “SDA polymerase”, as outlined below) and a mixture of all four deoxynucleoside-triphosphates (also called deoxynucleotides or dNTPs, i.e. dATP, dTTP, dCTP and dGTP), at least one species of which is a substituted or modified dNTP; thus, the SDA primer is modified, i.e. extended, to form a modified primer, sometimes referred to herein as a “newly synthesized strand”. The substituted dNTP is modified such that it will inhibit cleavage in the strand containing the substituted dNTP but will not inhibit cleavage on the other strand. Examples of suitable substituted dNTPs include, but are not limited, 2′deoxyadenosine 5′-O-(1-thiotriphosphate), 5-methyldeoxycytidine 5′-triphosphate, 2′-deoxyuridine 5′-triphosphate, adn 7-deaza-2′-deoxyguanosine 5′-triphosphate. In addition, the substitution of the dNTP may occur after incorporation into a newly synthesized strand; for example, a methylase may be used to add methyl groups to the synthesized strand. In addition, if all the nucleotides are substituted, the polymerase may have 5′→3′ exonuclease activity. However, if less than all the nucleotides are substituted, the polymerase preferably lacks 5′→3′ exonuclease activity. Once nicked, a polymerase (an “SDA polymerase”) is used to extend the newly nicked strand, 5′→3′, thereby creating another newly synthesized strand. The polymerase chosen should be able to intiate 5′→3′ polymerization at a nick site, should also displace the polymerized strand downstream from the nick, and should lack 5′→3′ exonuclease activity (this may be additionally accomplished by the addition of a blocking agent). Thus, suitable polymerases in SDA include, but are not limited to, the Klenow fragment of DNA polymerase I, SEQUENASE 1.0 and SEQUENASE 2.0 (U.S. Biochemical), T5 DNA polymerase and Phi29 DNA polymerase. Accordingly, the SDA reaction requires, in no particular order, an SDA primer, an SDA polymerase, a nicking endonuclease, and dNTPs, at least one species of which is modified.

In one embodiment, the target amplification technique is nucleic acid sequence based amplification (NASBA). A single stranded target nucleic acid, usually an RNA target sequence (sometimes referred to herein as “the first target sequence” or “the first template”), is contacted with a first NASBA primer. A “NASBA primer” generally has a length of 25100 nucleotides, with NASBA primers of approximately 50-75 nucleotides being preferred. The first NASBA primer is preferably a DNA primer that has at its 3′ end a sequence that is substantially complementary to the Tend of the first template. The first NASBA primer has an RNA polymerase promoter at its Fend. The first NASBA primer is then hybridized to the first template to form a first hybridization complex. The NASBA reaction mixture also includes a reverse transcriptase enzyme (an “NASBA reverse transcriptase”) and a mixture of the four dNTPs, such that the first NASBA primer is modified, i.e. extended, to form a modified first primer, comprising a hybridization complex of RNA (the first template) and DNA (the newly synthesized strand).

In one embodiment, the amplification technique is signal amplification. Signal amplification involves the use of limited number of target molecules as templates to either generate multiple signalling probes or allow the use of multiple signalling probes. Signal amplification strategies include LCR, CPT, and the use of amplification probes in sandwich assays.

In one embodiment, the devices include at least one fluid pump. Pumps generally fall into two categories: “on chip” and “off chip”; that is, the pumps (generally electrode based pumps) can be contained within the device itself, or they can be contained on an apparatus into which the device fits, such that alignment occurs of the required flow channels to allow pumping of fluids. In one embodiment, the pumps are contained on the device itself. These pumps are generally electrode based pumps; that is, the application of electric fields can be used to move both charged particles and bulk solvent, depending on the composition of the sample and of the device. Suitable on chip pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel to result in the pumping of the fluids comprising the sample components. As is described in the art, the configurations for each of these electrode based pumps are slighly different; for example, the effectiveness of an EHD pump depends on the spacing between the two electrodes, with the closer together they are, the smaller the voltage required to be applied to effect fluid flow. Alternatively, for EO pumps, the sampcing between the electrodes should be larger, with up to one-half the length of the channel in which fluids are being moved, since the electrode are only involved in applying force, and not, as in EHD, in creating charges on which the force will act. In one embodiment, an electroosmotic pump is used. Electroosmosis (EO) is based on the fact that the surface of many solids, including quartz, glass and others, become variously charged, negatively or positively, in the presence of ionic materials. The charged surfaces will attract oppositely charged counterions in aqueous solutions. Applying a voltage results in a migration of the counterions to the oppositely chaged electrode, and moves the bulk of the fluid as well. The volume flow rate is proportional to the current, and the volume flow generated in the fluid is also proportional to the applied voltage. Electroosmostic flow is useful for liquids having some conductivity is and generally not applicable for non-polar solvents. In one embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents. In one embodiment, a micromechanical pump is used, either on- or off chip, as is known in the art.

In one embodiment, an “off-chip” pump is used. For example, the devices may fit into an apparatus or appliance that has a nesting site for holding the device, that can register the ports (i.e. sample inlet ports, fluid inlet ports, and waste outlet ports) and electrode leads. The apparatus can including pumps that can apply the sample to the device; for example, can force cellcontaining samples into cell lysis modules containing protrusions, to cause cell lysis upon application of sufficient flow pressure. Such pumps are well known in the art.

In one embodiment, the devices include at least one fluid valve that can control the flow of fluid into or out of a module of the device, or divert the flow into one or more channels. In one embodiment, the devices include sealing ports, to allow the introduction of fluids, including samples, into any of the modules of the invention, with subsequent closure of the port to avoid the loss of the sample. In one embodiment, the devices include at least one storage modules for assay reagents. These are connected to other modules of the system using flow channels and may comprise wells or chambers, or extended flow channels. They may contain any number of reagents, buffers, enzymes, electronic mediators, salts, etc., including freeze dried reagents. In one embodiment, the devices include a mixing module; again, as for storage modules, these may be extended flow channels (particularly useful for timed mixing), wells or chambers. Particularly in the case of extended flow channels, there may be protrusions on the side of the channel to cause mixing.

One embodiment uses detection electrode comprises a self-assembled monolayer (SAM) comprising conductive oligomers. By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer. The SAM may comprise conductive oligomers alone, or a mixture of conductive oligomers and insulators. As outlined herein, the efficiency of target analyte binding (for example, oligonucleotide hybridization) may increase when the analyte is at a distance from the electrode. Similarly, nonspecific binding of biomolecules, including the target analytes, to an electrode is generally reduced when a monolayer is present. Thus, a monolayer facilitates the maintenance of the analyte away from the electrode surface. In addition, a monolayer serves to keep charged species away from the surface of the electrode. Thus, this layer helps to prevent electrical contact between the electrodes and the ETMs, or between the electrode and charged species within the solvent. Such contact can result in a direct “short circuit” or an indirect short circuit via charged species which may be present in the sample. Accordingly, the monolayer is preferably tightly packed in a uniform layer on the electrode surface, such that a minimum of “holes” exist. The monolayer thus serves as a physical barrier to block solvent accesibility to the electrode.

In one embodiment, electronic detection is used, including amperommetry, voltammetry, capacitance, and impedence. Suitable techniques include, but are not limited to, electrogravimetry; coulometry (including controlled potential coulometry and constant current coulometry); voltametry (cyclic voltametry, pulse voltametry, (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

In one embodiment, monitoring electron transfer is via amperometric detection. This method of detection involves applying a potential (as compared to a separate reference electrode) between the nucleic acid-conjugated electrode and a reference (counter) electrode in the sample containing target genes of interest. Electron transfer of differing efficiencies is induced in samples in the presence or absence of target nucleic acid; that is, the presence or absence of the target nucleic acid, and thus the label probe, can result in different currents.

The device for measuring electron transfer amperometrically involves sensitive current detection and includes a means of controlling the voltage potential, usually a potentiostat. This voltage is optimized with reference to the potential of the electron donating complex on the label probe. Possible electron donating complexes include those previously mentioned with complexes of iron, osmium, platinum, cobalt, rhenium and ruthenium being preferred and complexes of iron being most preferred.

Alternatively, the compositions are useful to detect successful gene amplification in PCR, thus allowing successful PCR reactions to be an indication of the presence or absence of a target sequence. PCR may be used in this manner in several ways. For example, in one embodiment, the PCR reaction is done as is known in the art, and then added to a composition comprising the target nucleic acid with a ETM, covalently attached to an electrode via a conductive oligomer with subsequent detection of the target sequence. Alternatively, PCR is done using nucleotides labelled with a ETM, either in the presence of, or with subsequent addition to, an electrode with a conductive oligomer and a target nucleic acid. Binding of the PCR product containing ETMs to the electrode composition will allow detection via electron transfer. Finally, the nucleic acid attached to the electrode via a conductive polymer may be one PCR primer, with addition of a second primer labelled with an ETM. Elongation results in double stranded nucleic acid with a ETM and electrode covalently attached. In this way, the system is used for PCR detection of target sequences.

In one embodiment, the arrays are used for mRNA detection. One embodiment utilizes either capture probes or capture extender probes that hybridize close to the 3′ polyadenylation tail of the mRNAs. This allows the use of one species of target binding probe for detection, i.e. the probe contains a poly-T portion that will bind to the poly-A tail of the mRNA target. Generally, the probe will contain a second portion, preferably non-poly-T, that will bind to the detection probe (or other probe). This allows one target-binding probe to be made, and thus decreases the amount of different probe synthesis that is done.

In one embodiment, the use of restriction enzymes and ligation methods allows the creation of “universal” arrays. In this embodiment, monolayers comprising capture probes that comprise restriction endonuclease ends. By utilizing complementary portions of nucleic acid, while leaving “sticky ends”, an array comprising any number of restriction endonuclease sites is made. Treating a target sample with one or more of these restriction endonucleases allows the targets to bind to the array. This can be done without knowing the sequence of the target. The target sequences can be ligated, as desired, using standard methods such as ligases, and the target sequence detected, using either standard labels or the methods of the invention.

As outlined herein, the devices can be used in combination with apparatus for delivering and receiving fluids to and from the devices. The apparatus can include a “nesting site” for placement of the device(s) to hold them in place and for registering inlet and outlet ports, if present. The apparatus may also include pumps (“off chip pumps”), and means for viewing the contents of the devices, including microscopes, cameras, etc. The apparatus may include electrical contacts in the nesting region which mate with contacts integrated into the structure of the chip, to power heating or electrophoresis, for example. The apparatus may be provided with conventional circuitry sensors in communication with sensors in the device for thermal regulation, for example for PCR thermal regulation. The apparatus may also include a computer system comprising a microprocessor for control of the various modules of the system as well as for data analysis.

FIG. 5B shows an exemplary flexible sensor array. Components such as resistors, capacitors and inductors can be printed on the flexible substrate as known by those skilled in the art. Transistors can also be printed. For high speed circuit, a hybrid using active electronics coupled to the flexible electronics can be used. Sensors can be built using these components. The substrate can be planar or non-planar. As used herein, the term “planar substrate” refers to a substrate which extends primarily in two dimensions, while the term “non-planar substrate” refers to a substrate that does not lie essentially in a two dimensional plane and can extend, for example, in a three dimensional orientation. For example, the substrate can include a three dimensional curved or angled (non-planar) housing of a mobile phone, game console, DVD player, computer, wireless modem, and the like. The substrate used with the system can be a planar and/or non-planar preformed molded plastic housing. The substrate also can be made from a variety of materials. Non-limiting examples of substrates include substrates made of acrylonitrile butadiene styrene (ABS), styrene acrylonitrile (SAN), polystyrene, polypropylene, high-density polyethylene (HDPE), low-density polyethylene (LDPE), polyamides, polysulfones, phenolic polymers, acrylics, vinyl polymers, glass, wood, urethanes, epoxies, polyesters, and mixtures thereof. The pores can be configured to form at least one conduit that opens to the outside of the surface of the substrate 13 or to the sensor 16 and extends to a location within the substrate 13 or all the way through the substrate 13. The pores can be any type of pores or pore system, or other similar configuration that allows for a substance to pass therethrough. The pores can be shaped, sized, and/or dimensioned to perform size exclusion selection on the substances that can pass therethrough. That is, the pores can be configured to restrict substances of a certain size from entering into the pores and/or passing from one surface of the substrate 13 to the opposite surface. Accordingly, the pores allow substances smaller than a certain size to enter into the pores. The size of the pores can be configured to be similar to the target substance, which can restrict access to the nanosensors and increase the accuracy of detection when the substrate is used for size exclusion selection. Non-limiting examples of pores sizes include being about, or less than about 0.1 nm, less than about 1 nm, less than about 10 nm, less than about 100 nm, less than about 1 um, less than about 10 um, and less than about 100 um. Additional non-limiting examples of pores sizes include being about 0.01 nm to about 0.1 nm, about 0.1 nm to about 1 nm, about 1 nm to about 10 nm, about 10 nm to about 100 nm, about 100 nm to about 1 um, about 1 um to about 10 um, and about 19 um to about 100 um.

A device can be formed of printed non-volatile memory on polymer. For example, the apparatus can be formed on a printed polymer integrated into packaging material and the integrated processor and memory can perform operations such as monitoring the number and type of touches of the product to determine marketing-relevant information such as attractiveness of the packaged material to consumers. The flexible device 1 can have a non-volatile memory array 11 and a processor 10 integrated with the flexible device 1. The processor 10 is operable to operate in combination with the non-volatile memory array 11 to accumulate information associated with a product. In various applications and contexts, memory systems can include non-volatile memory integrated with a processor or other control logic, and a bus or other communications interface. As non-volatile memories and integrated system continue to evolve, their role in overall systems continue to expand to include various aspects of computation that is facilitated, for example, by phase-change memory in which passage of current switches a memory material between two states, crystalline and amorphous, or additional states that further elevate storage capacity.

In some applications and/or embodiments, the processor 10 can be integrated with non-volatile memory array 11 to form the flexible device 1 which can be further integrated into the product, for example electronic devices, such as mobile and cell phones, notebook computers, personal digital assistants, medical devices, medical diagnostic systems, digital cameras, audio players, digital televisions, automotive and transportation engine control units, USB flash personal discs, and global positioning systems. Accordingly, the flexible device 1 can further include the product integrated with the non-volatile memory array 11 and the processor 10.

In embodiments of the apparatus with processing capability of a processor or other control logic integrated in a distributed manner with non-volatile memory, the processing capability can be implemented with relatively low speed requirement to enable processors to be available in a ubiquitous manner. Accordingly, information can be acquired in a dispersed manner and intercommunicated over vast systems. Thus processors can be inexpensive and memory readily available for various consumer items. Custom versions of memory including non-volatile memory and RAM can be integrated into virtually any product, enabling widespread preprocessing in items such as door handles to determine who has accessed a location and how the access was made to allow any type of processing on the information.

In some embodiments, the flexible device 1 can be configured such that the processor 10 is operable to accumulate and communicate information about use of the product. For example, the apparatus can be used in various types of medical devices to monitor and store aspects of operation. In a particular example embodiment, the apparatus can be used in medical products to form biocompatible electronic products such as electronic devices or medical support materials that can dissolve in a patient's body. Some medical products can be configured to be biocompatible and encapsulated in a textile material, silk, or other suitable substrate that dissolves after a selected time duration. The apparatus can also be constituted in a biodegradable form for implantation including biodegradable circuit components including transistors, diodes, inductors and capacitors that can dissolve in water or in the body.

In another example embodiment, the apparatus can be integrated into a product such as a vehicle, specifically a rental vehicle. For a rental automobile, the apparatus can be configured to monitor use such as distance, speed, or forces acting upon the automobile to ascertain driving behavior of the driver.

A further example application for use of the apparatus can be electrodes for a medical device, such as a Transcutaneous Electrical Nerve Stimulation (TENS) device or any other suitable device. A typical TENS system uses silver electrodes mounted on a fabric or cloth substrate. The apparatus including processor and memory can be integrated into the electrode for monitoring delivery of therapeutic pulses but also to monitor body signals such as electrical signals such as for diagnostic purposes. TENS devices produce electric current to stimulate the nerves for therapeutic purposes at a controlled or modulated pulse width, frequency and intensity. In various embodiments, the apparatus integrated into TENS electrodes includes processing capability that can enable chronic monitoring of biological electrical signals to facilitate diagnostic monitoring as well as therapeutic control.

In further applications and/or embodiments, the flexible device 1 can be constructed with the processor 10 operable to accumulate and communicate information about at least one entity in association with the product. In various embodiments and/or applications, an entity can be a person, a living being, a non-living being, an organization (business, political, or otherwise), a device, a computer, a network, or the like. For purposes of example, the apparatus can be integrated into a biocompatible, biodegradable form for hemodynamic monitoring of pressure and blood flow within the circulatory system. Thus, the processor and integrated memory in the apparatus can enable Holter monitoring of an ambulatory patient independently of any external device, although supporting communication with a device external to the patient's body via telemetry for exchange of commands, instructions, control information, and data.

In still further embodiments, the flexible device 1 can be formed in which the processor 10 is operable to accumulate and communicate information about at least one entity in communication with the product. For example, the apparatus can be integrated into a weather monitoring device such as a thermometer, barometer, anemometer, multi-meter that measures multiple environmental parameters, or the like. The weather monitoring device can include an apparatus that includes a communication interface and sensors integrated with the processor and memory. The weather monitoring device can be in a relatively inaccessible location and can communicate from this location to an entity, such as a weather computer or a person.

In additional example embodiments or applications, the flexible device 1 can be implemented so that the processor 10 is operable to accumulate and communicate information about at least one entity in contact with the product. For example, the apparatus can be integrated with a product in the form of a patient armband in hospitals, identification armband in workplaces or other locations, and the like, for instance to assist in security operations. In another example, the apparatus can be integrated with a product in the form of a soda can or other packaging, for example to assist in automatic or effortless purchase of the product.

In various embodiments, the flexible device 1 can be configured such that the processor 10 is operable to monitor tactile contact with the product. In some applications and/or conditions, tactile contact can be monitored via a tactile sensor accessed by the apparatus that can either be integrated into the apparatus, or the processor can be configured to accept tactile information from a distal sensor. In other applications, tactile information can be sent to the apparatus and processor. In example configurations, the apparatus can be integrated into a product in the form of a steering wheel, joystick, or other control device, and the control logic and memory can be configured to perform precision control operations. In another example embodiment, the apparatus can be integrated into a product in the form of a sports article such as a football, and the control logic and memory can be constructed to detect and identify a person with control of the product, such as identifying who has recovered a fumble.

In a particular example embodiment, the flexible device 1 can be constructed with the processor 10 operable to monitor tactile contact with the product, determine statistics on type, characteristics, and number of occurrences of tactile contact with the product, and store the statistics for access. For example, the apparatus can be integrated into a product in the form of a door handle or door handle sleeve. The processor and memory can be configured to monitor conditions such as who, what, when, and how many people have touched the door handle or sleeve. Some embodiments can monitor how hard the door handle or door handle sleeve is touched.

In various embodiments, the flexible device 1 can include volatile memory (not shown) in combination with the non-volatile memory array 11. Accordingly, in further applications or contexts for embodiments, the flexible device 1 can further include a volatile memory integrated with the non-volatile memory array 11 and the processor 10.

In one embodiment, a processor and flexible memory are integrated on a flexible printed polymer substrate and deployed into a multiple types of products. The device 1 can be composed such that the processor 10 and the non-volatile memory array 11 are integrated onto a printed flexible polymer for integration with the product. In one embodiment, the device 1 integrated onto a printed flexible polymer can be a product in the form of a medical device sleeve or patch, and the control logic and memory configured for use in monitoring implanted medical devices such as knee implants, hip implants, shoulder implants, elbow implants, and the like. The processor and memory can be configured to monitor aspects of performance such as position, angle, angular velocity or acceleration, other dynamics, and the like. In some arrangements, the processor and memory can be configured to assist physical therapy such as measurement of motion. In further arrangements, the processor and memory can be configured to monitor other biological or physiological functions such as blood flow, cardiac performance, hemodynamics, neurological aspects of action, and the like.

Accordingly, a flexible memory can be integrated with processors for further integration into any type of product, even very simple products such as bottles, cans, or packaging materials. A non-volatile memory can be integrated in a system of any suitable product such as, for example, a door handle sleeve to detect and record who, what, when, and how anyone has touched the door handle. Such a system can be used to facilitate access or to provide security. In other examples, a non-volatile memory and processor in some applications with sensors and/or a communication interface can be used in a flexible device for a medical product such as bandages or implants. These products can be formed of dissolvable materials for temporary usage, for example in biocompatible electronic or medical devices that can dissolve in a body environment, or environmental monitors and consumer electronics that can dissolve in compost. Other applications of products incorporating non-volatile memory and processor can include sporting equipment, tags such as for rental cars, patient armbands in hospitals tied to sensors, smart glasses, or any type of device.

In a particular example embodiment and application, the device 1 integrated as a printed flexible polymer can be used for cardiac monitoring such as in the form of a patch that can be attached to a patient's chest or elsewhere on the body. The processor and integrated memory can be used to control continuous monitoring of cardiac signals and activity. The device 1 can enable monitoring, such as by electrocardiography, independently of a separate medical device, although supporting communication and exchange of commands, instructions, and data with an external device.

In further embodiments, instead of a flexible polymer, the non-volatile memory and processor can be formed of silicon that is sufficiently thin to become flexible and thus formed as an inexpensive printed circuit component. Flexible memory in ubiquitous items, using polymer memory or silicon memory, can enable various profitable services, for example in conjunction with medical devices, security services, automotive products, and the like.

In an example embodiment, the apparatus can be integrated into a product in the form of smart glass, magic glass, switchable glass, smart windows or switchable windows for application in windows or skylights, which is electrically switchable glass or glazing which changes light transmission properties when voltage is applied. The apparatus can use the integrated processor and memory to control the amount of light and thus heat transmission. The processor can receive control commands, instructions, and data from a control center or operator, for example to activate the glass to change the glass between transparent and translucent, partially or fully blocking light while maintaining a clear view through the glass, if desired. In some embodiments, the communication interface can be used to report on conditions associated with the window or skylight.

The memory can be selected from a memory integrated circuit or memory chip, register, register file, random access memory (RAM), volatile memory, non-volatile memory, read-only memory, flash memory, ferroelectric RAM (F-RAM), magnetic storage device, disk, optical disk, and the like. In some arrangements, the memory can include multiple types of memory including the non-volatile memory array in the form of multiple types of non-volatile memory technologies, in addition to portions of memory that may be volatile. The memory may include multiple types of memory for use in a redundant fashion. Accordingly, the memory can include two or more memory segments of any non-volatile memory type or technology including read-only memory, flash memory, ferroelectric random access memory (F-RAM), magneto-resistive RAM (M-RAM) or the like. The processor or control logic can operate a segment of M-RAM which is comparable in speed and capacity to volatile RAM while enabling conservation of energy, rapid or instantaneous start-up and shutdown sequences. In other applications, the memory can include memory in the form of charge-coupled devices (CCDs) that are not directly addressable or other pure solid state memory that is reliable and inexpensive for use as separate memory for various applications such as cell phones, and the like.

In some embodiments and/or applications, the apparatus can further include a communication interface integrated with the processor and the non-volatile memory array. The communication interface can be operable for communication with a network. The processor can be operable to perform data preprocessing, history tracking, and manage data and history communication. For example, the apparatus can be integrated into a window and include one or more sensors and communication interface in combination with the processor and memory. The sensor(s) can include a light sensor, a pressure sensor, and a temperature sensor for use in determining conditions that can be monitored and communicated to enable control of a heating and cooling system of a building

In other embodiments, the apparatus can be integrated to a product in the form of a security device for securing an item such as a home, an automobile, or any other item of value. The apparatus can monitor conditions of the product autonomously of devices external to the product, while supporting updates to the apparatus.

For example, in some embodiments, the apparatus can include both phase change memory (PCRAM) and other memory types and the control logic can assign memory usage according to various operating characteristics such as available power. In a specific example, PCRAM and DRAM may be selected based on power considerations. PCRAM access latencies are typically in the range of tens of nanoseconds, but remain several times slower than DRAM. PCRAM writes use energy-intensive current injection, causing thermal stress within a storage cell that degrades current-injection contacts and limits endurance to hundreds of millions of writes per cell. In an apparatus that uses both PCRAM and DRAM, the control logic can allocate memory usage according to the write density of an application. In an apparatus that includes multiple different types of memory including a spin-transfer M-RAM, the control logic can assign functionality at least in part based on the magnetic properties of memory. In a system that includes at least one portion of F-RAM, the control logic can exploit operating characteristics of extremely high endurance, very low power consumption (since F-RAM does not require a charge pump like other non-volatile memories), single-cycle write speeds, and gamma radiation tolerance. The apparatus can include different segments of different types of memory including volatile and non-volatile memory, flash, dynamic RAM (DRAM) and the like, and use the control logic to attain different performance/cost benefits. In embodiments adapted to promote write durability, the apparatus can include a non-volatile memory array with multiple types of memory including at least one portion of memory characterized by elevated write endurance. In a particular embodiment, the non-volatile memory array can include at least on portion formed of M-RAM which is based on a tunneling magneto-resistive (TMR) effect. The individual M-RAM memory cells include a magnetic tunnel junction (MTJ) which can be a metal-insulator-metal structure with ferromagnetic electrodes. A small bias voltage applied between the electrode causes a tunnel current to flow. The MTJ is exposed to an external magnetic field and forms a hysteresis loop with two stable states, corresponding to 0 and 1 data states at zero magnetic field. M-RAM is characterized among non-volatile memory technologies as having excellent write endurance with essentially no significant degradation in magneto-resistance or tunnel junction resistance through millions of write cycles. Accordingly, the control logic can monitor and determine whether a particular application or process is characterized by frequent, enduring write operations and assign a portion of M-RAM to handle memory accesses. Another memory technology characterized by write endurance is ferroelectric RAM (FeRAM). FeRAM can be constructed using material such as lead-zirconate-titanate (PZT), strontium-bismuth-tantalate (SBT), lanthanum substituted bismuth-tantalate (BLT), and others. An externally applied electric field causes polarization of the FeRAM material to be switched and information retained even upon removal of the field. In absence of the electric field, polarization has two distinct stable states to enable usage in memory storage. FeRAM can have write endurance at the level of M-RAM and is further characterized by a reduced cell size and thus higher density. Thus, the control logic can monitor and determine whether a particular application or process is characterized by frequent, enduring write operations in combination with a relatively large number of storage cells. The control logic can assign a portion of FeRAM to handle memory accesses. The control logic can be a processor, a distributed-circuitry processor, a processing unit, a processing unit distributed over memory, arithmetic logic and associated registers, a microprocessor, a graphics processing unit, a physics processing unit, a signal processor, a network processor, a front-end processor, a state machine, a coprocessor, a floating point unit, a data processor, a word processor, and the like.

The apparatus can include any suitable type of sensor such as motion or position sensors, electrical signal sensors, pressure sensors, oxygen sensors, and the like. The processor and memory can be configured to facilitate monitoring for therapeutic and diagnostic purposes, and delivery of therapy. The control logic can be operable to perform maintenance operations including information handling in the memory in response to physical phenomena imposes on the memory. For example, the memory device can incorporate sensors or other components that detect phenomena which can be monitored by the control logic to detect magnetic fields, temperature, velocity, rotation, acceleration, inclination, gravity, humidity, moisture, vibration, pressure, sound, electrical fields or conditions such as voltage, current, power, resistance, and other physical aspects of the environment to enable the control logic to perform actions to maintain, repair, clean, or other operations applied to the memory.

In some embodiments and/or applications, the apparatus can receive information via the optical link, independently of the system bus connected to a processor, and the apparatus can use the extra-bus information to perform management or housekeeping functions to track applications and/or processes (or, for example, bit correction) via data sent optically to the apparatus. The optical link thus enables low-bandwidth, back-channel communication, enabling formation of a memory that can communicate with large bursts of data for placement with optical accessibility. For example, an optical sensor or silicon-based optical data connection can use silicon photonics and a hybrid silicon laser for communication between integrated circuit chips at distributed locations using plasmons (quanta of plasma oscillation) to communicate over relatively long distances, for example 2-3 inches on a narrow nano-wire coupler. The plasmon is a quasi-particle that results from quantization of plasma oscillations. Data can be received and converted using an optical antenna, a nano-cavity, or a quantum dot. The communication field can travel independently of a wired bus structure.

In some embodiments, the apparatus can be configured to respond to time signals. In various embodiments and/or arrangements, the time signal can be selected from among a visible, audible, mechanical, or electronic signal used as a reference to determine time, a clock, a timing pulse, and the like. Workload can refer to impact on the memory device, portions of memory within the memory device, the system containing the memory device, or any predetermined scope relative to the memory device, or the like. Workload can be analyzed and managed according to any selected workload parameters such as memory capacity, memory portion, memory type, memory characteristics, memory operating characteristics, memory availability, processor speed, logic speed, interface or network latency, potential workloads in queue, remaining battery life, energy cost, temperature, location, server type, affinity information, processing time, and the like.

Some embodiments can implement a pseudo-random number generator coupled to the hybrid memory and coupled to the logic operable to perform encryption operations. The pseudo-random number generator can be operable to generate numbers for usage in encrypting information. The medical information handling system can be configured to implement one or more of a variety of security schemes including channel encryption, storage encryption, RSA (Rivest, Shamir, Adleman) cryptography and key distribution, Public Key Infrastructure (PKI). Accordingly, the logic operable to perform encryption operations can be operable to perform stream encryption of communicated information wherein processor and memory sides are assigned a key. In another example functionality, the logic operable to perform encryption operations can be operable to encrypt information that is storage encrypted wherein the storage-encrypted information is encrypted by the processor, stored in the hybrid memory, accessed from the hybrid memory, and decrypted by the processor.

In some embodiments and/or applications, the information handling system can be configured to use of cryptographic processing to facilitate information handling. For example, data can be copied for redundant storage and the redundant copy can be secured by encryption and stored in the non-volatile memory in encrypted form. The encrypted redundant copy of the data can be used for restoration in the event of a detected error. In another example, A cryptographic hash function generates information indicative of data integrity, whether changes in data are accidental or maliciously and intentional. Modification to the data can be detected through a mismatching hash value. For a particular hash value, finding of input data that yields the same hash value is not easily possible, if an attacker can change not only the message but also the hash value, then a keyed hash or message authentication code (MAC) can supply additional security. Without knowing the key, for the attacker to calculate the correct keyed hash value for a modified message is not feasible.

In one embodiment, a humidity sensor employs a capacitor with a metal material such as copper or silver with a printed humidity sensitive polymer poly (2-hydroxyethyl methacrylate) (pHEMA). In this embodiment, the layer of pHEMA can be at the bottom, followed by the metal material, and by another layer of pHEMA. In another embodiment, the capacitor can be a silver or copper base with interdigitated arms formed above the base, and in this embodiment, the pHEMA is applied on one layer above the metal material. The sensor provides a capacitive response to the humidity. Various types of humidity-sensitive polymers containing doped cations or anions, quarternary ammonium, phosphonium salt and sulfonic acid-containing polyelectrolytes can be used for humidity sensing. Various conducting polymers such as polyaniline, polypyrrole and polythiophene can be used. Other materials include NaPSS: Sodium polystyrenesulfonate; DEAMA-co-BMA: Poly(N,N-diethylaminoethyl methacrylate-cobutyl methacrylate); MAPTAC: [3-(methacrylamino)propyl]trimethyl ammonium chloride; MSMA: 3-(trimethoxysilyl)propyl methacrylate; MMA: Methyl methacrylate; AEPAB: [2-(acryloyloxy)ethyl]dimethylpropyl ammonium bromide; PS: Polystyrene; HEMA: 2-hydroxyethylmethacrylate; BPA: 4-acryloyloxybenzophenone; PANI: Polyaniline; PVA: Polyvinyl alcohol: PSSA: Poly(styrenesulfonic acid); PVAc-co-BuAcry: Poly(vinyl acetate-cobutylacrylate); VTBPC: Vinylbenzyltributylphosphonium chloride; METAC: [2-(methacryloyloxy)ethyl]trimethyl ammonium chloride; 2-EHA: 2-ethylhexylacrylate; 4-VP: 4-vinylpyridine; MEDPAB: [2-(methacryloyloxy)ethyl]dimethylpropyl ammonium bromide; TSPM: 3-(trimethoxysilyl)propyl methacrylate; AMPS: Poly(2-acrylamido-2-methylpropane sulfonate); HMPTAC: 2-Hydroxy-3-methacryloxypropyltrimethylammonium chloride; PEG: Polyethylene glycol.

A printed temperature sensor can be a printed resistor with a positive temperature coefficient (PTC) or a negative temperature coefficient (NTC). To reduce impact of strain on the temperature sensor, in one embodiment, a temperature dependent resistor is formed in series with a temperature independent resistor, which is of similar construction and hence has a similar response to strain caused by mechanical force applied to a region of a sensing device including both resistors. By measuring variations in the potential difference across the temperature independent resistor, the mechanical distortion of the sensor can be determined. This information can be used to correct a measurement of the potential difference across the temperature dependent resistor, which indicates the change in temperature. Thus, in the case of a temperature sensor, the temperature reading of the sensor is automatically corrected for mechanical distortion (strain) of the sensor.

A touch sensor can be formed with a printed dielectric material layered between electrodes. While the touch sensor is illustrated as a single dielectric layered between two electrodes, it is to be understood that the touch sensor can include additional dielectric and electrode layers, depending on the design of the touch sensor. In an example, electrode can be the same material as electrode. In another example, electrode can be a different material from electrode. The dielectric and the electrodes can be formed of a polymer, such as a flexible polymer. The polymer may also be an amorphous polymer. In examples, the polymer can be a silicone, such as polydimethylsiloxane (PDMS). Furthermore, the electrodes can be a silicone and a conducting medium, such as carbon, or any other suitable conducting material, compounded into the silicone. When forming the touch sensor to a curved surface, regions of the touch sensor may deform more than other regions of the touch sensor, changing the capacitance of these deformed regions as compared to the less deformed regions of the touch sensor. By calibrating the touch sensor after forming the touch sensor to the curved surface, this change in capacitance can be negated. The touch sensor additionally supports a strain up to 400%, such as up to 350%. This high supported strain enables the force/deflection curve of the touch sensor to be made less sensitive when compared to a more rigid touchpad. In this sense, sensitivity relates to the force versus the deflection of the touch sensor. When a sensor is very stiff, a large force causes a small deflection in the sensor, making the sensor 200 very responsive to small deflections. This responsiveness to small deflection makes the input hard to control for the user. However, when the force is low and a large strain results due to the low modulus sensor material, the change of capacitance is large, resulting in a large signal input, so the user has greater control of the input signal by applying a force to the touch sensor (i.e., the sensor is less sensitive) and the touch sensor is less prone to errors. The capacitance of the touch sensor is changed by deforming the touch sensor. In some cases, deforming the touch sensor means applying pressure to the touch sensor such that the shape of the touch sensor is altered. Capacitance is a function of the electrode area A, the electrode charge, the distance d between electrodes, and the permittivity of the volume between charge plates. When a force is exerted on the touch sensor, the electrode area A deforms and the distance d changes, which in turn changes the capacitance of the touch sensor. The capacitance is sensed by a circuit (not illustrated) and correlated to a force applied to the touch sensor.

The device 1 can be powered by a flexible battery such as lithium-ion battery with a negative electrode, or anode, and a positive electrode, or cathode, coated on a metal foil current collector. Between these electrodes is a thin polymer separator, which keeps the electrodes from touching and allows lithium ions to pass though during charging or discharging. The metal foil current collectors are formed as Chemical Vapor Deposition (CVD)-grown carbon nanotube mats. Carbon nanotubes are highly conductive and extremely strong-two features a flexible battery would need in order to generate power in diverse forms. A separator is placed between a carbon nanotube-based anode and cathode that they then encapsulated in a thin, flexible plastic film.

The exemplary clothing of FIG. 1A has flexible circuits thereon. Accelerometers, temperature sensors, EKG sensors, EMG sensors, and other sensors can be formed on the flexible clothing. One major symptom of a stroke is unexplained weakness or numbness in the muscle. To detect muscle weakness or numbness, in one embodiment, the system applies a pattern recognizer such as a neural network or a Hidden Markov Model (HMM) to analyze accelerometer output. In another embodiment, electromyography (EMG) is used to detect muscle weakness. In another embodiment, EMG and a pattern analyzer is used to detect muscle weakness. In yet another embodiment, a pattern analyzer analyzes both accelerometer and EMG data to determine muscle weakness. In a further embodiment, historical ambulatory information (time and place) is used to further detect changes in muscle strength. In yet other embodiments, accelerometer data is used to confirm that the patient is at rest so that EMG data can be accurately captured or to compensate for motion artifacts in the EMG data in accordance with a linear or non-linear compensation table. In yet another embodiment, the EMG data is used to detect muscle fatigue and to generate a warning to the patient to get to a resting place or a notification to a nurse or caregiver to render timely assistance. The amplitude of the EMG signal is stochastic (random) in nature and can be reasonably represented by a Gausian distribution function. The amplitude of the signal can range from 0 to 10 mV (peak-to-peak) or 0 to 1.5 mV (rms). The usable energy of the signal is limited to the 0 to 500 Hz frequency range, with the dominant energy being in the 50-150 Hz range. Usable signals are those with energy above the electrical noise level. The dominant concern for the ambient noise arises from the 60 Hz (or 50 Hz) radiation from power sources. The ambient noise signal may have an amplitude that is one to three orders of magnitude greater than the EMG signal. There are two main sources of motion artifact: one from the interface between the detection surface of the electrode and the skin, the other from movement of the cable connecting the electrode to the amplifier. The electrical signals of both noise sources have most of their energy in the frequency range from 0 to 20 Hz and can be reduced. To eliminate the potentially much greater noise signal from power line sources, a differential instrumentation amplifier can be attached to the flexible substrate. Any signal that originates far away from the detection sites will appear as a common signal, whereas signals in the immediate vicinity of the detection surfaces will be different and consequently will be amplified. Thus, relatively distant power lines noise signals will be removed and relatively local EMG signals will be amplified. The source impedance at the junction of the skin and detection surface may range from several thousand ohms to several megohms for dry skin. In order to prevent attenuation and distortion of the detected signal due to the effects of input loading, the input impedance of the differential amplifier is as large as possible, without causing ancillary complications to the workings of the differential amplifier. The signal to noise ratio is increased by filtering between 20-500 Hz with a roll-off of 12 dB/octave.

In one embodiment, direct EMG pre-amplification at the skin surface provides the best myoelectric signal quality for accurate, reliable EMG signal detection and eliminates cable motion artifact. The double-differential instrumentation pre-amplifier design attenuates unwanted common-mode bioelectric signals to reduce cross-talk from adjacent muscle groups. Internal RFI and ESD protection prevents radio frequency interference and static damage. The constant low-impedance output of the pre-amplifier completely eliminates cable noise and cable motion artifacts without requiring any additional signal processing within the pre-amplifier. An integral ground reference plane provides immunity to electromagnetic environmental noise. All signal and power conductors in the pre-amplifier cable are enclosed inside an independent, isolated shield to eliminate interference from AC power-lines and other sources. The contacts are corrosion-free, medical grade stainless steel for maximal signal flow. The system uses biocompatible housing and sensor materials to prevent allergic reactions.

In another implementation, a micro-powered EMG embodiment includes an instrumentation amplifier and an AC coupling that maintains a high CMRR with a gain of about 1000. The electronic circuits are mounted on a flexible circuit board (FPC) with slidable electrode settings that allows differential recording at various distances between the electrodes. The high gain amplifier is placed next to the recording electrodes to achieve high SNR. Battery power provides isolation and low noise at various frequencies that would likely not be fully attenuated by the PSRR and causing alias errors.

The system can detect dominant symptoms of stroke can include weakness or paralysis of the arms and/or legs, incoordination (ataxia), numbness in the arms/legs using accelerometers or EMG sensors. The EMG sensors can detect muscle fatigue and can warn the patient to get to a resting area if necessary to prevent a fall. The system can detect partial/total loss of vision by asking the patient to read a predetermined phrase and detect slur using speech recognizer. The system can detect loss of consciousness/coma by detecting lack of movement. Voice/speech disturbances are not initially the dominant symptoms in stroke, and the disturbances can be detected by a speech recognizer. In one implementation, the system uses PNL (probabilistic networks library) to detect unusual patient movement/ambulatory activities that will lead to a more extensive check for stroke occurrence. PNL supports dynamic Bayes nets, and factor graphs; influence diagrams. For inference, PNL supports exact inference using the junction tree algorithm, and approximate inference using loopy belief propagation or Gibbs sampling. Learning can be divided along many axes: parameter or structure, directed or undirected, fully observed or partially observed, batch or online, discriminative or maximum likelihood, among others. First, the system performs data normalization and filtering for the accelerometers and EMG sensors that detect patient movements and muscle strength. The data can include in-door positioning information, 3D acceleration information, or EMG/EKG/EEG data, for example. The data can be processed using wavelet as discussed above or using any suitable normalization/filtering techniques. Next, the system performs parameterization and discretization. The Bayesian network is adapted in accordance with a predefined network topology. The system also defines conditional probability distributions. The system then generates the probability of event P(y), under various scenarios. Training data is acquired and a training method is built for the Bayesian network engine. Next, the system tunes model parameters and performs testing on the thus formed Bayesian network.

In one embodiment, a housing (such as a strap, a wrist-band, or a patch) provides a plurality of sensor contacts for EKG and/or EMG. The same contacts can be used for detecting EKG or EMG and can be placed as two parallel contacts (linear or spot shape) on opposite sides of the band, two adjacent parallel contacts on the inner surface of the band, two parallel adjacent contacts on the back of the wrist-watch, or alternatively one contact on the back of the watch and one contact on the wrist-band. The outputs of the differential contacts are filtered to remove motion artifacts. The differential signal is captured, and suitably filtered using high pass/low pass filters to remove noise, and digitized for signal processing. In one embodiment, separate amplifiers are used to detect EKG (between 50 mHz and 200 Hz) and for EMG (between 10 Hz and 500 Hz). In another embodiment, one common amp is used for both EKG/EMG, and software filter is applied to the digitized signal to extract EKG and EMG signals, respectively. The unit can apply Wavelet processing to convert the signal into the frequency domain and apply recognizers such as Bayesian, NN or HMM to pull the EMG or EKG signals from noise. The system uses a plurality of wireless nodes to transmit position and to triangulate with the mobile node to determine position. 3D accelerometer outputs can be integrated to provide movement vectors and positioning information. Both radio triangulation and accelerometer data can confirm the position of the patient. The RF signature of a plurality of nodes with known position can be used to detect proximity to a particular node with a known position and the patient's position can be extrapolated therefrom.

In one embodiment, Analog Device's AD 627, a micro-power instrumentation amplifier, is used for differential recordings while consuming low power. In dual supply mode, the power rails Vs can be as low as ±1.1 Volt, which is ideal for battery-powered applications. With a maximum quiescent current of 85 μA (60 μA typical), theunit can operate continuously for several hundred hours before requiring battery replacement. The batteries are lithium cells providing 3.0 V to be capable of recording signals up to +1 mV to provide sufficient margin to deal with various artifacts such as offsets and temperature drifts. The amplifier's reference is connected to the analog ground to avoid additional power consumption and provide a low impedance connection to maintain the high CMRR. To generate virtual ground while providing low impedance at the amplifier's reference, an additional amplifier can be used. In one implementation, the high-pass filtering does not require additional components since it is achieved by the limits of the gain versus frequency characteristics of the instrumentation amplifier. The amplifier has been selected such that with a gain of 60 dB, a flat response could be observed up to a maximum of 100 Hz with gain attenuation above 100 Hz in one implementation. In another implementation, a high pass filter is used so that the cut-off frequency becomes dependent upon the gain value of the unit. The bootstrap AC-coupling maintains a much higher CMRR so critical in differential measurements. Assuming that the skin-electrode impedance may vary between 5 K- and 10 K-ohms, 1 M-ohm input impedance is used to maintain loading errors below acceptable thresholds between 0.5% and 1%.

When an electrode is placed on the skin, the detection surfaces come in contact with the electrolytes in the skin. A chemical reaction takes place which requires some time to stabilize, typically in the order of a few seconds. The chemical reaction should remain stable during the recording session and should not change significantly if the electrical characteristics of the skin change from sweating or humidity changes. The active electrodes do not require any abrasive skin preparation and removal of hair. The electrode geometry can be circular or can be elongated such as bars. The bar configuration intersects more fibers. The inter detection-surface distance affects the bandwidth and amplitude of the EMG signal; a smaller distance shifts the bandwidth to higher frequencies and lowers the amplitude of the signal. An inter detection-surface of 1.0 cm provides one configuration that detects representative electrical activity of the muscle during a contraction. The electrode can be placed between a motor point and the tendon insertion or between two motor points, and along the longitudinal midline of the muscle. The longitudinal axis of the electrode (which passes through both detection surfaces) should be aligned parallel to the length of the muscle fibers. The electrode location is positioned between the motor point (or innervation zone) and the tendinous insertion, with the detection surfaces arranged so that they intersect as many muscle fibers as possible.

In one embodiment, a multi-functional bio-data acquisition provides programmable multiplexing of the same differential amplifiers for extracting EEG (electroencephalogram), ECG (electrocardiogram), or EMG (electromyogram) waves. The system includes an AC-coupled chopped instrumentation amplifier, a spike filtering stage, a constant gain stage, and a continuous-time variable gain stage, whose gain is defined by the ratio of the capacitors. The system consumes microamps from 3V. The gain of the channel can be digitally set to 400, 800, 1600 or 2600. Additionally, the bandwidth of the circuit can be adjusted via the bandwidth select switches for different biopotentials. The high cut-off frequency of the circuit can be digitally selected for different applications of EEG acquisition.

In another embodiment, a high-resolution, rectangular, surface array electrode-amplifier and associated signal conditioning circuitry captures electromyogram (EMG) signals. The embodiment has a rectangular array electrode-amplifier followed by a signal conditioning circuit. The signal conditioning circuit is generic, i.e., capable of receiving inputs from a number of different/interchangeable EMG/EKG/EEG electrode-amplifier sources (including from both monopolar and bipolar electrode configurations). The electrode-amplifier is cascaded with a separate signal conditioner minimizes noise and motion artifact by buffering the EMG signal near the source (the amplifier presents a very high impedance input to the EMG source, and a very low output impedance); minimizes noise by amplifying the EMG signal early in the processing chain (assuming the electrode-amplifier includes signal gain) and minimizes the physical size of this embodiment by only including a first amplification stage near the body. The signals are digitized and transmitted over a wireless network such as WiFI, Zigbee, or Bluetooth transceivers and processed by the base station that is remote from the patient. For either high-resolution monopolar arrays or classical bipolar surface electrode-amplifiers, the output of the electrode-amplifier is a single-ended signal (referenced to the isolated reference). The electrode-amplifier transduces and buffers the EMG signal, providing high gain without causing saturation due to either offset potentials or motion artifact. The signal conditioning circuit provides selectable gain (to magnify the signal up to the range of the data recording/monitoring instrumentation, high-pass filtering (to attenuate motion artifact and any offset potentials), electrical isolation (to prevent injurious current from entering the subject) and low-pass filtering (for anti-aliasing and to attenuate noise out of the physiologic frequency range).

The EMG signal can be rectified, integrated a specified interval of and subsequently forming a time series of the integrated values. The system can calculate the root-mean-squared (rms) and the average rectified (avr) value of the EMG signal. The system can also determine muscle fatigue through the analysis of the frequency spectrum of the signal. The system can also assess neurological diseases which affect the fiber typing or the fiber cross-sectional area of the muscle. Various mathematical techniques and Artificial Intelligence (AI) analyzer can be applied. Mathematical models include wavelet transform, time-frequency approaches, Fourier transform, Wigner-Ville Distribution (WVD), statistical measures, and higher-order statistics. AI approaches towards signal recognition include Artificial Neural Networks (ANN), dynamic recurrent neural networks (DRNN), fuzzy logic system, Genetic Algorithm (GA), and Hidden Markov Model (HMM).

A single-threshold method or alternatively a double threshold method can be used which compares the EMG signal with one or more fixed thresholds. The embodiment is based on the comparison of the rectified raw signals and one or more amplitude thresholds whose value depends on the mean power of the background noise. Alternatively, the system can perform spectrum matching instead of waveform matching techniques when the interference is induced by low frequency baseline drift or by high frequency noise.

EMG signals are the superposition of activities of multiple motor units. The EMG signal can be decomposed to reveal the mechanisms pertaining to muscle and nerve control. Decomposition of EMG signal can be done by wavelet spectrum matching and principle component analysis of wavelet coefficients where the signal is de-noised and then EMG spikes are detected, classified and separated. In another embodiment, principle components analysis (PAC) for wavelet coefficients is used with the following stages: segmentation, wavelet transform, PCA, and clustering. EMG signal decomposition can also be done using non-linear least mean square (LMS) optimization of higher-order cumulants.

Time and frequency domain approaches can be used. The wavelet transform (WT) is an efficient mathematical tool for local analysis of non-stationary and fast transient signals. One of the main properties of WT is that it can be implemented by means of a discrete time filter bank. The Fourier transforms of the wavelets are referred as WT filters. The WT represents a very suitable method for the classification of EMG signals. The system can also apply Cohen class transformation, Wigner-Ville distribution (WVD), and Choi-Williams distribution or other time-frequency approaches for EMG signal processing.

In Cohen class transformation, the class time-frequency representation is particularly suitable to analyze surface myoelectric signals recorded during dynamic contractions, which can be modeled as realizations of nonstationary stochastic process. The WVD is a time-frequency that can display the frequency as a function of time, thus utilizing all available information contained in the EMG signal. Although the EMG signal can often be considered as quasi-stationary there is still important information that is transited and may be distinguished by WVD. Implementing the WVD with digital computer requires a discrete form. This allows the use of fast Fourier transform (FFT), which produces a discrete-time, discrete-frequency representation. The common type of time frequency distribution is the Short-time Fourier Transform (STFT). The Choi-Williams method is a reduced interference distribution. The STFT can be used to show the compression of the spectrum as the muscle fatigue. The WVD has cross-terms and therefore is not a precise representation of the changing of the frequency components with fatigue. When walls appear in the Choi-William distribution, there is a spike in the original signal. It will decide if the walls contain any significant information for the study of muscle fatigue. In another embodiment, the autoregressive (AR) time series model can be used to study EMG signal. In one embodiment, neural networks can process EMG signal where EMG features are first extracted through Fourier analysis and clustered using fuzzy c-means algorithm. Fuzzy c-means (FCM) is a method of clustering which allows data to belong to two or more clusters. The neural network output represents a degree of desired muscle stimulation over a synergic, but enervated muscle. Error-back propagation method is used as a learning procedure for multilayred, feedforward neural network. In one implementation, the network topology can be the feedforward variety with one input layer containing 256 input neurodes, one hidden layer with two neurodes and one output neurode. Fuzzy logic systems are advantageous in biomedical signal processing and classification. Biomedical signals such as EMG signals are not always strictly repeatable and may sometimes even be contradictory. The experience of medical experts can be incorporated. It is possible to integrate this incomplete but valuable knowledge into the fuzzy logic system, due to the system's reasoning style, which is similar to that of a human being. The kernel of a fuzzy system is the fuzzy inference engine. The knowledge of an expert or well-classified examples are expressed as or transferred to a set of “fuzzy production rules” in the form of IF-THEN, leading to algorithms describing what action or selection should be taken based on the currently observed information. In one embodiment, higher-order statistics (HOS) is used for analyzing and interpreting the characteristics and nature of a random process. The subject of HOS is based on the theory of expectation (probability theory).

In addition to stroke detection, EMG can be used to sense isometric muscular activity (type of muscular activity that does not translate into movement). This feature makes it possible to define a class of subtle motionless gestures to control interface without being noticed and without disrupting the surrounding environment. Using EMG, the user can react to the cues in a subtle way, without disrupting their environment and without using their hands on the interface. The EMG controller does not occupy the user's hands, and does not require them to operate it; hence it is “hands free”. The system can be used in interactive computer gaming which would have access to heart rate, galvanic skin response, and eye movement signals, so the game could respond to a player's emotional state or guess his or her level of situation awareness by monitoring eye movements. EMG/EEG signal can be used for man-machine interfaces by directly connecting a person to a computer via the human electrical nervous system. Based on EMG and EEG signals, the system applies pattern recognition system to interpret these signals as computer control commands. The system can also be used for Mime Speech Recognition which recognizes speech by observing the muscle associated with speech and is not based on voice signals but EMG. The MSR realizes unvoiced communication and because voice signals are not used, MSR can be applied in noisy environments; it can support people without vocal cords and aphasics. In another embodiment, EMG and/or electroencephalogram (EEG) features are used for predicting behavioral alertness levels. EMG and EEG features were derived from temporal, frequency spectral, and statistical analyses. Behavioral alertness levels were quantified by correct rates of performance on an auditory and a visual vigilance task, separately. A subset of three EEG features, the relative spectral amplitudes in the alpha (alpha %, 8-13 Hz) and theta (theta %, 4-8 Hz) bands, and the mean frequency of the EEG spectrum (MF) can be used for predicting the auditory alertness level.

In yet a further embodiment for performing motor motion analysis, an HMM is used to determine the physical activities of a patient, to monitor overall activity levels and assess compliance with a prescribed exercise regimen and/or efficacy of a treatment program. The HMM may also measure the quality of movement of the monitored activities. For example, the system may be calibrated or trained in the manner previously described, to recognize movements of a prescribed exercise program. Motor function information associated with the recognized movements may be sent to the server for subsequent review. A physician, clinician, or physical therapist with access to patient data may remotely monitor compliance with the prescribed program or a standardized test on motor skill. For example, patients can take the Wolf Motor Function test and acceleration data is captured on the following tasks:

placing the forearm on a table from the side

moving the forearm from the table to a box on the table from the side

extending the elbow to the side

extending the elbow to the side against a light weight

placing the hand on a table from the front

moving the hand from table to box

flexing the elbow to retrieve a light weight

lifting a can of water

lifting a pencil, lifting a paper clip

stacking checkers, flipping cards

turning a key in a lock

folding a towel

lifting a basket from the table to a shelf above the table.

The suit of FIG. 1A can have a diaper with flexible circuits thereon. In one embodiment, the diaper receives a deposit of capacitive or resistive sensors that detect soiling and communicating the amount of soiling via RF means to a monitoring station for diaper change. Urine Handling with microneedles as one way valves is detailed next. In addition to a conventional diaper with superabsorbent crystals, microneedle valves are used to minimize backflow and odor. Urine flows down microneedles as a urine catcher. The urine then passes through a sealing liquid, such as a designed oil based fluid or vegetable oil, and collects in the reservoir below. The different densities of urine and oil (urine is denser than oil—oil floats!) mean that the urine sinks through the sealing liquid and the oil floats on top of the layer of urine below. Any air bubbles rise to the top and escape leaving the urine in a relatively low oxygen environment. Odor is therefore trapped below the oil layer and odor is eliminated. Preferably, the system is designed to slow the urine before it hits the oil so that laminar flow displacement doesn't move the oil to the bottom. After catching the urine in the reservoir, an outlet is provided to dispose urine into the toilet plumbing system. In one embodiment, to increase urine capacity, multiple urine tanks can be formed around the body of the underwear and a pump can be used to move the urine to different tanks for balance. Each tank includes a drain outlet that is joined at a master outlet so that a single valve can be used to dispose urine into the toilet plumbing system. There are two embodiments: cartridge based and non-cartridge based units. Cartridge based units use a replaceable cartridge pre-filled with sealing liquid. These units are periodically replaced as the sealing liquid is slowly eroded or degraded. Non Cartridge based systems work by simply introducing the sealing liquid into the drain hole and allowing it to naturally settle into the correct position.

In yet another implementation, the odor trapping is controlled electronically using a liquid detector and valve or clamp that is opened when urination is detected but otherwise is closed. In one implementation, a pump can be used to move urine into a storage chamber embedded in the front or back regions to provide high storage capacity. The urine chamber has an electronically controlled discharge valve so the user can wirelessly dump the urine without touching the urine container when the user subsequently visits a toilet. The user can also manually discharge the urine if the wireless control is not available or if needed for any reasons.

To handle fecal matters, a disposable biodegradable pad is placed under the anus, and an expandable container or bellow is used to capture fecal matters. When not needed, the bellow is compressed into a small volume. During use, the bellow expands to capture the fecal matters. When the session is done, the wearer moves to a toilet, uses the hand in a wiping motion to clean him/herself and at the end of the motion the liner/bag is released into the toilet. Thus, in one action, the pad with the accordion bag is disposed while the body is cleansed. When the fecal is a solid, cleaning is easy. However, when the fecal matter is liquid or chunky, cleaning is quite challenging. To actively capture liquid fecal matters, a pump is used to suck the liquid into the bellow/container. Upon detection of liquid exiting the anus, the pump is activated and causes the pad to form a seal around butt and to suck the liquid fecal into the accordion disposable bellow. In another embodiment that is quieter than the pump embodiment, to provide an electrostatic force that delivers liquid fecal matter into the bellow container, the pad can be negatively charged, while the bellow can be positively charged. In another embodiment, both the pump and the electrostatic differential can be used to forcefully urge liquid fecal matter to into the accordion like bellow container. An active directed movement of liquid fecal matter when the wearer is about to have a bowel movement minimizes skin rash and other medical problem if the skin is exposed to waste materials for an extended duration.

In one embodiment, an odor control dispenser such as fragrance fluid dispenser or solid dispenser can be activated to neutralize odor at the point of use. A highly concentrated plant extract can be used to avoid polluting the environment with eucalyptus, floral oasis and refreshing spring flavors.

In another embodiment, the fecal storage pad and bellow container, including the other parts identified above, is biodegradable or, preferably, formed from a substance that may dissolve or disintegrate in water so that the fecal and the entire chamber may be flushed in the toilet after use. Samples of such material include, paper and other cellulosic materials, materials formed substantially from starch, gum, or alginate material such as agar and so on.

In one embodiment, a user facing layer may be formed of Duratex™, which is an aperture film with a non-woven scrim (AFW/NW) layer attached. Aperture film has small holes in it the shape of a funnel, which helps to move fluid in only one direction. Non-woven fibers passing over the holes of the aperture film, and the film is oriented such that the apertures point away from the body, allowing fluid to pass into the lower layers, but not to return. A second layer uses a through air bonded (TAB) material similar to bleeder/breather that is used in the composite industry and allows nesting of the apertures and the spreading of fluids to the manifold. A third layer, an aperture film, is the start of the manifold. A porcupine type roller may be used to form the aperture film for forming the number of holes, or such holes may be punched or otherwise machine formed. The number of holes may be varied to determine optimum performance of the apparatus. A fourth layer forms the center of the manifold and may comprise either TAB or bleeder/breather, a polyester non-woven fabric. The density of material may be increased around the tube exit area. In any event, the manifold nests in this material. The last layer is an outside layer back sheet that can be a treated breathable sheet or breathable polyethylene (PE) film. The edges of the article may be sealed together by heat bonding, melting adhesive (e.g., hot glue), air stitch, or other methods.

A processor or CPU detects urine liquid by determining two electrodes are shorted when the urine flows through the electrodes. The CPU activates a clamp to allow the urine to flow into a reservoir 103. When urine is not present, the claim returns to its normally closed position to cut off odor and urine from getting out of the reservoir. Subsequently, when the user is at a toilet, the user can wirelessly instruct the CPU 101 to open the urine out valve to dump the urine into the toilet. The command can be from a smart phone, smart watch, or smart wearable device that transmits the command over WiFi, Bluetooth, Zigbee, or other wired media, for example. Once the command is received, the urine reservoir content is dumped out and the reservoir can be reused again to relieve the user when needed, yet avoiding dumping diaper into the landfill each time s/he urinates with a diaper.

A transceiver provides a portable wireless incontinence monitoring system for aged care facilities. Benefits of remote monitoring include increasing quality of life for the elderly and reducing the work load of caregivers. The system detects and accurately measures the voided volume for each event. A strip with an array of sensors is placed in a diaper to measure conductivity of urine. Sensors capture volume sizes, timing between each event and the number of urinary events per day. In one embodiment, an incontinence monitoring system includes a sensor placed into the article, and connected to the system of FIG. 1C which is placed in the patients' underwear. The wireless transceiver 107 transmits the sensors' data to a server which collects all the data from all in an aged care facility. The recorded data is then analyzed by software and the results are shown to the end user via a user interface. The caregivers can check the residents' status from any workstation in communication with the server to see if the resident has to be changed or not. Also, an alert can be sent to a caregiver's mobile telephone, tablet computer or other mobile communication device. The system provides a process for caregivers to take care of residents while maintaining user comfort. Caregivers need to only create a profile for each resident with the user interface via any workstation. This enables the system to keep track of each resident and alert the caregivers when a resident's underwear or diaper has to be changed.

In another embodiment, a camera can be used to capture patient data. For a stool analysis, a stool sample is collected in the container and analyzed by camera and sensor(s). The camera analysis includes microscopic examination, chemical tests, and microbiologic tests. The stool is checked for color, consistency, weight (volume), shape, odor, and the presence of mucus. The stool may be examined for hidden (occult) blood, fat, meat fibers, bile, white blood cells, and sugars called reducing substances.

Human fecal matter varies significantly in appearance, depending on diet and health. In one embodiment, the camera classifies stools using the Bristol stool scale which is a medical aid designed to classify the form of human feces into seven categories. Developed by K. W. Heaton at the University of Bristol, the seven types of stool are: Separate hard lumps, like nuts (hard to pass), Sausage-shaped but lumpy, Like a sausage but with cracks on the surface, Like a sausage or snake, smooth and soft, Soft blobs with clear-cut edges, Fluffy pieces with ragged edges, a mushy stool, and Watery, no solid pieces. Entirely Liquid. Types 1 and 2 indicate constipation. Types 3 and 4 are optimal, especially the latter, as these are the easiest to pass. Types 5-7 are associated with increasing tendency to diarrhea or urgency.

In one embodiment, the camera checks for the color of the stool as follows:

Brown: Human feces ordinarily has a light to dark brown coloration, which results from a combination of bile and bilirubin that is derived from dead red blood cells. Normally it is semisolid, with a mucus coating.

Yellow: Yellowing of feces can be caused by an infection known as Giardiasis, which derives its name from Giardia, an anaerobic flagellated protozoan parasite that can cause severe and communicable yellow diarrhea. Another cause of yellowing is a condition known as Gilbert's Syndrome. Yellow stool can also indicate that food is passing through the digestive tract relatively quickly. Yellow stool can be found in people with GERD gastroesophageal reflux disease.

Pale or Clay: Stool that is pale or grey may be caused by insufficient bile output due to conditions such as cholecystitis, gallstones, giardia parasitic infection, hepatitis, chronic pancreatitis, or cirrhosis. Bile salts from the liver give stool its brownish color. If there is decreased bile output, stool is much lighter in color.

Black or Red: Feces can be black due to the presence of red blood cells that have been in the intestines long enough to be broken down by digestive enzymes. This is known as melena, and is typically due to bleeding in the upper digestive tract, such as from a bleeding peptic ulcer. Conditions that can also cause blood in the stool include hemorrhoids, anal fissures, diverticulitis, colon cancer, and ulcerative colitis. The same color change can be observed after consuming foods that contain a substantial proportion of animal blood, such as black pudding or tiôtcanh. Black feces can also be caused by a number of medications, such as bismuth subsalicylate (the active ingredient in Pepto-Bismol), and dietary iron supplements, or foods such as beetroot, black liquorice, or blueberries. Hematochezia is similarly the passage of feces that are bright red due to the presence of undigested blood, either from lower in the digestive tract, or from a more active source in the upper digestive tract. Alcoholism can also provoke abnormalities in the path of blood throughout the body, including the passing of red-black stool.

Blue: Prussian blue, used in the treatment of radiation, cesium, and thallium poisoning, can turn the feces blue. Substantial consumption of products containing blue food dye, such as blue curacao or grape soda, can have the same effect.

Silver: A tarnished-silver or aluminum paint-like feces color characteristically results when biliary obstruction of any type (white stool) combines with gastrointestinal bleeding from any source (black stool). It can also suggest a carcinoma of the ampulla of Vater, which will result in gastrointestinal bleeding and biliary obstruction, resulting in silver stool.

Green: Feces can be green due to having large amounts of unprocessed bile in the digestive tract. This can occasionally be the result from eating liquorice candy, as it is typically made with anise oil rather than liquorice herb and is predominantly sugar. Excessive sugar consumption or a sensitivity to anise oil may cause loose, green stools.

Purple: Purple feces is a symptom of porphyria.

In another embodiment, an electronic nose is used to detect feces possess physiological odor, which can vary according to diet (especially the amount of meat protein e.g., methionine and health status. The odor of human feces is suggested to be made up from the following odorant volatiles:

Methyl sulfides: methylmercaptan/methanethiol (MM), dimethyl sulfide (DMS), dimethyl trisulfide (DMTS), dimethyl disulfide (DMDS)

Benzopyrrole volatiles: indole, skatole

Hydrogen sulfide (H2S)

(H2S) is the most common volatile sulfur compound in feces. The odor of feces may be increased in association with various pathologies, including: Celiac disease, Crohn's disease, ulcerative colitis, chronic pancreatitis, cystic fibrosis, intestinal infection, clostridium difficile infection, malabsorption, short bowel syndrome.

The system can also control odor through UV light or chemicals such as bismuth subsalicylate, chloryphyllyn, herbs such as rosemary, yucca schidigera, zinc acetate.

In other embodiments, the pH of the stool also may be measured. A stool culture is done to find out if bacteria may be causing an infection. Other stool analytics can be done to:
Help identify diseases of the digestive tract, liver, and pancreas. Certain enzymes (such as trypsin or elastase) may be evaluated in the stool to help determine how well the pancreas is functioning.
Help find the cause of symptoms affecting the digestive tract, including prolonged diarrhea, bloody diarrhea, an increased amount of gas, nausea, vomiting, loss of appetite, bloating, abdominal pain and cramping, and fever.
Screen for colon cancer by checking for hidden (occult) blood.
Look for parasites, such as pinworms or Giardia lamblia.
Look for the cause of an infection, such as bacteria, a fungus, or a virus.
Check for poor absorption of nutrients by the digestive tract (malabsorption syndrome).

The electronic nose can have a sensor array, composed of a plurality of sensors, disposed within a cavity of the excrement container, each sensor for measuring the different variety of compounds within the gas sample. The number of arrays is limited by power consumption design requirements. In a preferred embodiment, two identical sensor arrays are disposed within the first cavity. Using multiple identical sensor arrays provides at least the following benefits; 1) fault tolerance methods for increased reliability can be employed; 2) enables a more accurate measurement of the sample is possible through the use of sensor array averaging methods; and 3) various error correction algorithms can be implemented. Each of the at least one sensor arrays measures properties of the gas sample and produces an output, which is received by a CPU (central processing unit) or processor in signal communication with each of the at least one sensor arrays, the processor for receiving the output and controlling operation of the at least one sensor array. The plurality of sensors used in each of the at least one sensor arrays can be of low-cost, non-selective commercial type gas sensors. For example, a hybrid structure array with a plurality of MOS, and/or MOSFET, and/or CP, and/or SAW and/or QCM, VOC gas sensors can be utilized. Ideally, each of the at least one sensor arrays should be composed of at least four different gas target and/or sensor type gas sensors as well as one temperature sensor and one humidity sensor in order to increase compound selectivity and response. Many manufacturers use different sensing technologies that generate different responses. It has been shown that comparative methods using responses from more types of sensors provide better overall results. In a preferred embodiment, one sensor array is positioned on an upper wall of the first cavity, and a second sensor array is positioned on a lower wall of the first cavity. It should be noted that there are various techniques such as temperature modulation and compound filtering that can be applied to the sensors and the gas sample in order to generate many virtual sensors from only a small number of physical sensors within each of the at least one sensor arrays. Since sensor performance improves at higher temperatures, a second heater may be utilized to heat the first cavity. For each sensor, the temperature of MOS film affects the kinetics of the adsorption and reaction processes that take place within the sensor. Also, in the presence of multiple compounds, each will react preferentially as the temperature of the sensor varies. In the same way, the higher temperatures within the first cavity may impact compound separation from each gas sample and facilitate better selective response from the sensors. Since temperature impacts the measurements it is beneficial to be able to modulate and control the temperature of both the sensors and the first cavity itself. For this reason, additional heaters (not shown) may be associated with each sensor array.

The camera can have image processing capability to detect diarrhea, bloody diarrhea. Other sensors can be used to detect an increased amount of gas, nausea, vomiting, loss of appetite, bloating, abdominal pain and cramping, and fever. The fecal elastase test is another test of pancreas function. The test measures the levels of elastase, an enzyme found in fluids produced by the pancreas. Elastase digests (breaks down) proteins. A fecal occult blood test can be used to diagnose many conditions that cause bleeding in the gastrointestinal system including colorectal cancer or stomach cancer. Parasitic diseases such as ascariasis, hookworm, strongyloidiasis and whipworm can be diagnosed by examining stools under a microscope for the presence of worm larvae or eggs. Some bacterial diseases can be detected with a stool culture. Toxins from bacteria such as Clostridium difficile can also be identified. Viruses such as rotavirus can also be found in stools. A fecal pH test may be used determine lactose intolerance or the presence of an infection. Steatorrhea can be diagnosed using a Fecal fat test that checks for the malabsorption of fat. Faecalelastase levels are becoming the mainstay of pancreatitis diagnosis

One test checks for pinworms, a type of roundworm. The roundworms are classified as parasites with microscopic eggs. Adults measure anywhere from five to ten centimeters. A camera is used to detect eggs and moving worms.

Another test detects colon cancer. Over 100,000 persons per year in the United States are afflicted with cancer of the colon and rectum. When the number of colon/rectal cancers occurring each year is combined with the number of cancers occurring in other digestive organs, including the esophagus and stomach, such cancers of the digestive system account for more occurrences of cancer than any other single form of the disease. Contrary to many other forms of cancer, early diagnosis and treatment of digestive tract cancer does result in a cure rate of 80% to 90%. If, however, the disease is not detected until the later stages, the cure rate drops significantly. Thus, early detection of the disease is important to successful treatment of digestive tract cancer. Most, but not all, cancers of the digestive tract bleed to a certain extent. This blood is deposited on and in fecal matter excreted from the digestive system. The presence of blood in fecal matter is not normally detected, however, until gross bleeding, that is, blood visible to the naked eye, occurs. Gross bleeding, however, is symptomatic of advanced cancers. Digestive tract cancers in the early stages, including pre-cancerous polyps, also tend to bleed, giving rise to occult (hidden) blood in the fecal matter. Other pathological conditions, such as Crohn's disease and diverticulitis, can also give rise to the presence of occult blood in the fecal matter.

Certain embodiments include diagnostic capability such as those for colorectal screening which save lives as a result. The embedded diagnostic in these embodiments provides a private and convenient means for preliminarily detecting fecal blood. Upon detecting blood, individuals are more likely to consult a health care physician for a colorectal screening. The test material is formed from biodegradable material or material that easily disintegrates in water so that the kit may be toilet disposed without exposing individuals to infectious micro-organisms.

One test that can be done is disclosed in Pagano U.S. Pat. No. 3,996,006, which is incorporated herein by reference in its entirety. In general, the Pagano test employs an absorbent paper impregnated with a guaiac reagent and encased in a special test slide having openable flaps on both sides of the test slide. A sample of fecal matter contacts the guaiac impregnated paper and a nonaqueous developing solution is applied to the guaiac impregnated paper. If occult blood is present in the fecal matter on the opposite side of the paper, the guaiac reaction will dye the paper blue, providing a positive indication of the presence of blood in the fecal matter.

In another occult blood test embodiment, the stool is mixed with a compound which, when present in an aqueous solution with at least one of blood, blood fractions, blood components and hemoglobin, results in a chemiluminescence. In further embodiments, the compound undergoes a reaction in aqueous solution which is catalyzed by at least one of blood, blood fractions, blood components and hemoglobin. In further embodiments, the reaction is catalyzed by the hem iron of hemoglobin. The system includes a luminescent, preferably dry luminol (C8H7N3O2), which may be packaged and contained in a container 22. Some compounds related to luminol such as: Luminol, hemihydrate; Luminol, Na salt; Luminol, HCL; isoluminol; isoluminol, monohydrate; and isoluminol ABEI, to name some examples, may be more or less suitable. Luminol may be synthesized using known means beginning from 3-nitrophthalic acid. First, hydrazine (N2H4) is heated with the 3-nitrophthalic acid in a high-boiling solvent such as triethylene glycol. Nitrophthalhydrazide is formed by a condensation reaction. Reduction of the nitro group on the Nitrophthalhydrazide yields luminol. To exhibit its luminescence, an amount of water (oxidant) sufficient to produce a mixture of the luminescent and sample is added. The lid may then be placed on the open end of the container and the contents swirled, shaken, or otherwise sufficiently mixed to thoroughly mix the aqueous solution with the sample. In one embodiment, the chemiluminescent compound undergoes a light-producing reaction which involves, as a reactant or catalyst, blood or blood components or products. In another embodiment, the chemiluminescent compound is luminol or a related compound, such as the examples listed above, which undergoes a luminescence-producing reaction in the container which is catalyzed by blood components, particularly the iron component of whole hemoglobin. In the presence of iron, which is found in the hemoglobin of blood, and which functions as a catalyst, the luminol will luminesce.

Yet other non-invasive diagnostic methods involve assaying stool samples for the presence of fecal occult blood or for elevated levels of carcinoembryonic antigen, both of which are suggestive of the presence of colorectal cancer. Additionally, techniques for detecting the presence of a range of DNA mutations or alterations associated with and indicative of the presence of colorectal cancer can be used. The presence of such mutations can be detected in DNA found in stool samples during the early stages of colorectal cancer. As cells and cellular debris are shed from colonic epithelial cells onto forming stool in a longitudinal “stripe” of material along the length of the stool, the system can take a representative sample in order to ensure that the sample will contain any cells or cellular debris that was shed into the stool as it passed through the colon. Accordingly, the system obtains a representative (e.g. a cross-section or circumferential surface) portion of stool voided by a patient, and performing an assay to detect in the sample the presence of cells or cellular debris shed from epithelial cells lining the colon that may be indicative of cancer or precancer. Most often, such cells will be derived from a polyp or a cancerous or precancerous lesion at a discrete location along the colon. For purposes of the present invention, a precancerous lesion comprises precancerous cells, and precancerous cells are cells that have a mutation that is associated with cancer and which renders such cells susceptible to becoming cancerous. A cross-sectional sample is a sample that contains at least a circumferential surface of the stool (or portion of a stool comprising an entire cross-sectional portion), as, for example, in a coronal section or a sagittal section. A sample comprising the surface layer of a stool (or of a cross-section of a stool) also contains at least a circumferential surface of the stool. Both cross-sections and circumferential surfaces comprise longitudinal stripes of sloughed colonic epithelium, and are therefore representative samples.

The housing (and the urine collector) can be cleaned with a UV light cleaning accessory. In one embodiment, “ultraviolet light” or simply “ultraviolet (UV)” is applied. UV is the electromagnetic radiation emitted from the region of the spectrum lying beyond the visible light and before x-rays. The upper wavelength limit is 400 nanometers (1 nm=10-g meter) and the lower wavelength limit is 100 nm, below which radiation ionizes virtually all molecules. The region between 400 and 190 nm has been divided into three regions: NEAR-ultraviolet radiation or UV-A can be considered to lie in the wavelength range 320-400 nm. The long wavelength limit represents the beginning of the visible spectrum, while the short wavelength limit corresponds roughly to the point below which proteins and genetic material begin to absorb significantly. Below this region is the MID-UV region or UV-B (290320 nm), where proteins and genetic material begin to absorb and where sunburn and skin cancer are most effectively produced. (UV radiation present in sunlight at the surface of the earth at noon in clear weather includes both the NEAR-UV and the MID-UV regions.) FAR-UV (UV-C) wavelengths range from 200-290 nm, and because of their strong absorption by genetic material, are highly destructive to biological matter. These wavelengths are almost all absorbed by the ozone in the stratosphere. The wavelength of ultraviolet light produced by the UV lamps which are used for the disinfection of water is 254 nm, which is in the FAR-UV or UV-C range.

The narrow band of UV light lying between the wavelengths of 200 and 300 nm has often been called the germicidal region because UV light in this region is lethal to microorganisms including: bacteria, protozoa, viruses, molds, yeasts, fungi, nematode eggs and algae. The most destructive wavelength is 260 nm which is very close to the wavelength of 254 nm produced by germicidal lamps. UV light's ability to kill the fecal coliform bacteria, Escherichia coli, is directly related to the ability of its genetic material (i.e. nucleic acid) to absorb UV light. UV light causes molecular rearrangements in the genetic material of microorganisms and this prevents them from reproducing. Most microorganisms have relatively short life cycles and therefore depend on rapid reproduction to sustain and grow their population. Therefore, if a microorganism cannot reproduce then it is considered to be dead. Normally when DNA replicates, the Thymine (T) must join the Adenine (A), and the Cytosine (C) must join with Guanine (G). When DNA is exposed to Ultraviolet Light at a wavelength of 254 nm, an error occurs in the replication process. The Thymine forms a dimer, that is, a double bond between the Thymine molecules. This error prevents the pathogen from reproducing properly and so eventually it dies off.

One embodiment is an exemplary airbag with a cartridge that activates when the accelerometer detects that the user is falling and needs cushioning. While only one set is shown, it is understood that as many sets can be used as desired. For example, four sets can be spaced apart on the front, back, and sides of the user to provide 360 degree protection.A sensor such as an angle change sensor, an altitude sensor, a G-force decrease sensor or other sensor recognizes a characteristic change that accompanies a fall. The processor actuates a compressed air (or other gas) chamber, and an air bag to each set. A release valve can be actuated for compressed air chamber to rapidly release its contents to air bag for full deployment. On/off switch may be utilized to deactivate the module so that a wearer may change any characteristic without setting off the air bags. In other words, the device can be turned on and off as desired, e.g., a motorcyclist can turn it on when embarking and shut it off when disembarking.

In other embodiments, the air bag can be in the form of a vest device. The vest device has a front bag and a supply and control module well as a rear bag and a supply and control module. Once a fall is detected, the air bags are deployed in time to create a soft fall. In one embodiment, the front air bag blows up to support the chin and neck but not to shut off breathing, while rear air bag extends up the back of the neck and the back of the head to protect both the neck and the back of the head. In another embodiment, a set of separate jacket and pants present invention air bag can be used. Here jacket has a hood with a head back air bag system (this system includes at least one air bag and at least one module), arms with air bags. There is also a front chest bag. Pants includes hip units, such as left hip air bag, as well as leg units, such as leg air bag. The jacket and pants function in a manner the same as described above. In other embodiments, attachment means such as belt strap and latch and corresponding belt strap and buckle are exemplary and can be used to attach to a torso, back or buttocks. Alternatively, it could be in the form of a belt and attached to a waist. The shock buffering protection will be activated immediately upon a fall detection to release gas such as CO 2 gas into the neck, cheeks, body, back and hip airbags to inflate them in a brief time such as 0.5 second to reduce the impact of the fall.

In one embodiment, the printed flexible fall detector can be smart clothing with a microcontroller with accelerometers, gyroscopes, and magnetometers. Optionally, the fall detector can have a vertical detector to detect if the patient is on the ground. In other embodiment, the detection of height can be done using an accelerometer, where the accelerometer will be dropped in one translation down from the height to the earth. For rotational, the accelerometer will drop, but it will also have a spin to it, and a rotation. With linear, rotational and projectile falls, the system can determine the height of the fall by sampling by knowing the rate that the accelerometer is sampled by the microcontroller, the time that an object starts to fall,and the time that impact occurs. This gives a difference equal to the time of the fall. This information can be taken with an equation to determine the height of the fall. The fall detector can be a tag in a standalone mode that actuates the gas generator using an electrically actuated pin that punctures the gas cannister. The fall detector can also be a portable consumer device such as a smart phone. Either can work alone, and for improved detection accuracy, the fall detector can employ software on both the tag and the smart phone. In one embodiment, the application (App) can be downloaded from a store to the phone. The App can be put into “test mode” where the user can see which motions trigger the “alarm” and which don't. The app will have some “thresholds” that will need to be set to “optimize” performance. If a fall is detected and the sensors detect that the user cannot get up within a predetermined time, the phone can make outgoing calls to a sequence of emergency contacts, including a call center, family telephone number, caregiver telephone number, and other helper's telephone number if a fall is detected. Voice, text or email messages can be sent. The user will be able to override an emergency phone call by manually cancelling the call. Text messages will not be able to be cancelled.

One exemplary system provides for monitoring urination and/or defecation and reporting the event to staff or a caregiver for assistance. Embodiments provide information regarding the nature and volume of exudate associated with a wetness event and more particularly, the volume of individual events in a sequence of events occurring during the wearing of an absorbent pad. This information is useful to be able to determine the frequency, type and severity of each incontinence episode suffered by an individual and developing an incontinence profile in order to prescribe a suitable treatment or management plan for the individual's incontinence. The system can then determine when the total amount of exudate absorbed by an absorbent pad is approaching or has reached the limit of the pad's absorbent capacity and whether changing of the pad is required. The system can determine whether an absorbent pad is likely to require changing without necessarily requiring manual periodic checking of the pad by staff in a care facility.

The system can work with the sensors discussed above. Alternatively, for a conventional diaper, the system can work with an exudate sensor that includes a pad body, one or more wings formed at two sides of the pad body, a top layer, a cover layer and multiple capacitive or resistive wetness sensors in the cover layer. In one embodiment, in lieu of the resistive/capacitive sensors, a humidity sensor detects humidity around the diaper and determines the urine volume in the diaper. A separate capacitive or resistive fecal sensor is placed so that it is underneath the anus during use to detect the extrusion of fecal matters. In another embodiment, the processor receives the wetness information from the capacitive or resistive sensors and estimates the volume of urine received by the diaper so far and if capacity has been reach, signals the staff or caregiver to change the diaper and clean the patient. The volume estimation is done by detecting which grid had a short and the length of the short. The system knows the area of each grid, and by integrating the areas that had shorts caused by the resistive elements in the grid array, the system can estimate the volume of urine secreted. If the volume exceeds a predetermined volume which is greater than a minor leak, the system alerts caregivers to change clothing and clean the user. The processor can compare the estimated volume with a pre-defined threshold level. If the estimated volume is less than the threshold, the processor continues to monitor the sensor signals. If the estimated volume exceeds the threshold amount, then the processor sends an alert to a caregiver (carer). Once a carer is alerted, the carer attends to the resident and may choose to change the absorbent article and the processor detects that the sensor has been disconnected from the system and resets the sensor data. The threshold volume used by the processor to alert a carer may be a “qualifying amount” e.g. indicated as small, medium or large or a quantifying amount being a pre-defined volume e.g. 50 ml.

Preferably, the processor may also execute an algorithm to compare the estimated volume with a known estimated capacity of the diaper to give carers an indication of when the diaper is likely to become saturated with exudate so that it can be changed before a saturating wetness event occurs and the patient is made to feel uncomfortable by excess wetness.

The processor may also monitor the total amount of accumulated moisture in a series of wetness events in a single absorbent article and provide an indication to a carer as to when the absorbent capacity of the garment has been or is likely to be reached, to prompt the carer to change the garment for the patient's comfort and wellbeing.

Users may enter data, including patient specific demographic data such as gender, age, height and weight via user interface. Other entered data may include medical data, i.e. medication, amount of fluid and food intake, details of known conditions, recent surgeries, years in assisted care, years wearing an incontinence garment, continence function if known, and mental condition.

The processor may be incorporated into a central monitoring station such as a nurse's station. The processor may also integrate with or be incorporated into existing nurse call and remote patient monitoring systems controlled at the nurse's station. The processor may also be integrated with other care management systems for streamlining access to non-sensor related data contained within other care management systems such as, for example, fluid and food intake, patient relocation, showering, toileting, surgeries etc.

User interface may also include a transmitter which sends alerts to communication devices such as pagers or nurse phones carried by carers to indicate that there has been a wetness event, or that one is due to occur, or that physical inspection of the patient is required or due. In addition to the detection of wetness events which are estimated to exceed a threshold amount, these conditions warranting physical inspection may include when exudate is fecal in nature or when sensors detect blood, a parasite or a biological or chemical marker in the urine or faeces.

In one embodiment, observation data is used, along with a log of the sensor signals received at the input, to identify patterns in the patent's continence activity. The processor derives automatically, using an algorithm employing another mathematical model, a continence care plan based on the pattern, i.e. frequency and repetition of monitored events. The care plan includes a voiding or toileting schedule which statistically predicts wetness events based on the observed pattern. This is used by carers to plan the regularity (e.g. times of day) that a patient is to be manually checked for wetness and/or assisted with toileting and to plan when to empty the bladder or bowel, prior to periods in which a patient is known to have a pattern of incontinence events. Normal care of the patient can then take place without the need to continually monitor using a sensor.

The voiding schedule anticipates when a wetness event is statistically likely to occur and this can be used to automatically generate an audible and/or visible alert for a carer (e.g. presented on a screen of the user interface 108 or transmitted to a pager or the like) to attend to the patient by assisting with manual toileting or to change the patient's incontinence garment.

It is recommended that the toileting/voiding schedule is re-evaluated periodically (step 310) to maintain its accuracy, in keeping with changes in the patient's continence patterns. Re-evaluation may take place for example every 3, 6 or 12 months, or whenever actual wetness events do not correspond well with those anticipated by the voiding schedule.

In another use of the invention, the moisture monitoring system includes a log for recording wetness events detected by sensors including the volume, time and nature (urinary and/or fecal) of each event. These data are used to produce a bladder diary. These data may also be combined with details entered e.g. at the user interface 108 which relate to food and fluid intake (amount, kind and time), toileting and also any particular activities that the patient has undertaken.

The log may manifest in a memory device in communication or integrated with the processor. The processor may be located centrally and receive sensor signals relating indicative of wetness of a number of absorbent articles worn by different patients. Alternatively, there may be a pre-processor executing the algorithm located near the sensor, on the absorbent article. That is, the sensor and the part of the processor performing the analysis may be a provided together with the sensor. In such arrangement, the pre-processor may also incorporate a transmitter for transmitting data from the pre-processor to e.g. a central monitoring system which may include a display.

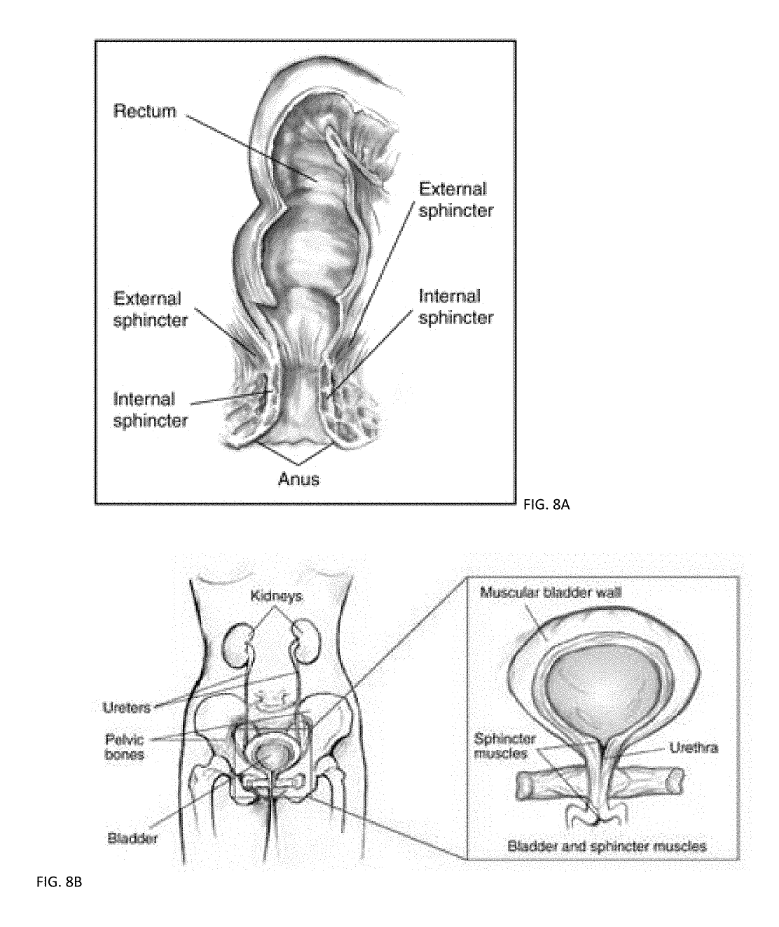

FIGS. 8A and 8B illustrate two embodiments for bowel control and bladder control with FES. Sacral stimulators can be surgically implanted for on-demand control of the bladder or bowel. Alternatively, electrical stimulation signals can be beamed or directed at particular muscles of the bladder and bowel. In other embodiments, upon detecting an unwanted bladder movement, the controller can actuate the FES to counter the unwanted bladder opening.

In one embodiment, FES is used to control incontinence which occurs because of problems with muscles and nerves that help to hold or release urine. The body stores urine-water and wastes removed by the kidneys-in the bladder, a balloon-like organ. The bladder connects to the urethra, the tube through which urine leaves the body. During urination, muscles in the wall of the bladder contract, forcing urine out of the bladder and into the urethra. At the same time, sphincter muscles surrounding the urethra relax, letting urine pass out of the body. Incontinence will occur if your bladder muscles suddenly contract or the sphincter muscles are not strong enough to hold back urine. Urine may escape with less pressure than usual if the muscles are damaged, causing a change in the position of the bladder. FES is used to work with the sphincter muscles to hold the urine.

In one embodiment, bowel control relies on FES control of muscles and nerves of the rectum and anus working together to hold stool in the rectum or release stool when the person is ready. FES is used to stimulate circular muscles called sphincters to close tightly like rubber bands around the anus until stool is ready to be released and at that point FES drives the sphincters to open the anus. Pelvic floor muscles are also controlled by FES and also help with bowel control. Sacral nerve stimulation or neuromodulation, involves placing electrodes near the sacral nerves to the anus and rectum and continuously stimulating the nerves with electrical pulses. The sacral nerves connect to the part of the spine in the hip area.

Functional neuromuscular stimulation of the respiratory muscles can restore inspiratory and respiratory functions. Functional electrical stimulation can restore breathing to patients with spinal cord injury or those needing life support.

In one embodiment, the EMG sensors can be embedded with the stimulators. The device records voluntary EMG from a pair of surface stimulation electrodes for functional electrical stimulation (FES). The device can apply to a small muscle on which it is difficult to locate both the stimulation electrodes and recording electrodes. The device utilizes photo-MOS relays to disconnect the stimulator when it is not active, to ground the electrodes after delivering the stimulation pulses, and to drop the gain of the EMG amplifier during the stimulus period. The device can detect the voluntary EMG of a small muscle from the stimulation electrodes for the EMG-controlled FES system.

The EMG sensors can be used alone or with other sensors such as EKG, BI, BIA, GSR, EEM, among others as detailed below.

One embodiment includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the patient body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies.

In a tetrapolar implementation, two electrodes on the wrist watch or wrist band are used to apply AC or DC constant current into the body or body segment. The voltage signal from the surface of the body is measured in terms of impedance using the same or an additional two electrodes on the watch or wrist band. In a bipolar implementation, one electrode on the wrist watch or wrist band is used to apply AC or DC constant current into the body or body segment. The voltage signal from the surface of the body is measured in terms of impedance using the same or an alternative electrode on the watch or wrist band. The system may include a BI patch that wirelessly communicates BI information with the wrist watch. Other patches 1400 can be used to collect other medical information or vital parameter and communicate with the wrist watch or base station or the information could be relayed through each wireless node or appliance to reach a destination appliance such as the base station, for example. The system can also include a head-cap 1402 that allows a number of EEG probes access to the brain electrical activities, EKG probes to measure cranial EKG activity, as well as BI probes to determine cranial fluid presence indicative of a stroke. As will be discussed below, the EEG probes allow the system to determine cognitive status of the patient to determine whether a stroke had just occurred, the EKG and the BI probes provide information on the stroke to enable timely treatment to minimize loss of functionality to the patient if treatment is delayed.

Bipolar or tetrapolar electrode systems can be used in the BI instruments. Of these, the tetrapolar system provides a uniform current density distribution in the body segment and measures impedance with less electrode interface artifact and impedance errors. In the tetrapolar system, a pair of surface electrodes (I1, I2) is used as current electrodes to introduce a low intensity constant current at high frequency into the body. A pair of electrodes (E1, E2) measures changes accompanying physiological events. Voltage measured across E1-E2 is directly proportional to the segment electrical impedance of the human subject. Circular flat electrodes as well as band type electrodes can be used. In one embodiment, the electrodes are in direct contact with the skin surface. In other embodiments, the voltage measurements may employ one or more contactless, voltage sensitive electrodes such as inductively orcapacitively coupled electrodes. The current application and the voltage measurement electrodess in these embodiments can be the same, adjacent to one another, or at significantly different locations. The electrode(s) can apply current levels from 20 uA to 10 mA rms at a frequency range of 20-100 KHz. A constant current source and high input impedance circuit is used in conjunction with the tetrapolar electrode configuration to avoid the contact pressure effects at the electrode-skin interface.

The BI sensor can be a Series Model which assumes that there is one conductive path and that the body consists of a series of resistors. An electrical current, injected at a single frequency, is used to measure whole body impedance (i.e., wrist to ankle) for the purpose of estimating total body water and fat free mass. Alternatively, the BI instrument can be a Parallel BI Model In this model of impedance, the resistors and capacitors are oriented both in series and in parallel in the human body. Whole body BI can be used to estimate TBW and FFM in healthy subjects or to estimate intracellular water (ICW) and body cell mass (BCM). High-low BI can be used to estimate extracellular water (ECW) and total body water (TBW). Multi-frequency BI can be used to estimate ECW, ICW, and TBW; to monitor changes in the ECW/BCM and ECW/TBW ratios in clinical populations. The instrument can also be a Segmental BI Model and can be used in the evaluation of regional fluid changes and in monitoring extra cellular water in patients with abnormal fluid distribution, such as those undergoing hemodialysis. Segmental BI can be used to measure fluid distribution or regional fluid accumulation in clinical populations. Upper-body and Lower-body BI can be used to estimate percentage BF in healthy subjects with normal hydration status and fluid distribution.The BI sensor can be used to detect acute dehydration, pulmonary edema (caused by mitral stenosis or left ventricular failure or congestive heart failure, among others), or hyperhydration cause by kidney dialysis, for example. In one embodiment, the system determines the impedance of skin and subcutaneous adipose tissue using tetrapolarand bipolar impedance measurements. In the bipolar arrangement the inner electrodes act both as the electrodes that send the current (outer electrodes in the tetrapolar arrangement) and as receiving electrodes. If the outer two electrodes (electrodes sending current) are superimposed onto the inner electrodes (receiving electrodes) then a bipolar BIA arrangement exists with the same electrodes acting as receiving and sending electrodes. The difference in impedance measurements between the tetrapolar and bipolar arrangement reflects the impedance of skin and subcutaneous fat. The difference between the two impedance measurements represents the combined impedance of skin and subcutaneous tissue at one or more sites. The system determines the resistivities of skin and subcutaneous adipose tissue, and then calculates the skinfold thickness (mainly due to adipose tissue).

Various BI analysis methods can be used in a variety of clinical applications such as to estimate body composition, to determine total body water, to assess compartmentalization of body fluids, to provide cardiac monitoring, measure blood flow, dehydration, blood loss, wound monitoring, ulcer detection and deep vein thrombosis. Other uses for the BI sensor includes detecting and/or monitoring hypovolemia, hemorrhage or blood loss. The impedance measurements can be made sequentially over a period of in time; and the system can determine whether the subject is externally or internally bleeding based on a change in measured impedance. The watch can also report temperature, heat flux, vasodilation and blood pressure along with the BI information.

In one embodiment, the BI system monitors cardiac function using impedance cardiography (ICG) technique. ICG provides a single impedance tracing, from which parameters related to the pump function of the heart, such as cardiac output (CO), are estimated. ICG measures the beat-to-beat changes of thoracic bioimpedance via four dual sensors applied on the neck and thorax in order to calculate stroke volume (SV). By using the resistivity p of blood and the length L of the chest, the impedance change ΔZ and base impedance (Zo) to the volume change ΔV of the tissue under measurement can be derived as follows:

$$Δ V = ρ \frac{{L}^{2}}{{Z}_{0}^{2}} Δ Z$$

In one embodiment, SV is determined as a function of the first derivative of the impedance waveform (dZ/dtmax) and the left ventricular ejection time (LVET)

$$SV = ρ \frac{{L}^{2}}{{Z}_{0}^{2}} {( \frac{dZ}{dt} )}_{max} LVET$$

In one embodiment, L is approximated to be 17% of the patient's height (H) to yield the following:

$$SV = ( \frac{{( 0.17 H )}^{3}}{4.2} ) \frac{{( \frac{dZ}{dt} )}_{max}}{{Z}_{0}} LVET$$

In another embodiment or the actual weight divided by the ideal weight is used:

$$SV = δ \times ( \frac{{( 0.17 H )}^{3}}{4.2} ) \frac{{( \frac{dZ}{dt} )}_{max}}{{Z}_{0}} LVET$$

The impedance cardiographic embodiment allows hemodynamic assessment to be regularly monitored to avoid the occurrence of an acute cardiac episode. The system provides an accurate, noninvasive measurement of cardiac output (CO) monitoring so that ill and surgical patients undergoing major operations such as coronary artery bypass graft (CABG) would benefit. In addition, many patients with chronic and comorbid diseases that ultimately lead to the need for major operations and other costly interventions might benefit from more routine monitoring of CO and its dependent parameters such as systemic vascular resistance (SVR).

Once SV has been determined, CO can be determined according to the following expression:

CO=SV *HR, where HR=heart rate

CO can be determined for every heart-beat. Thus, the system can determine SV and CO on a beat-to-beat basis.

In one embodiment to monitor heart failure, an array of BI sensors are place in proximity to the heart. The array of BI sensors detect the presence or absence, or rate of change, or body fluids proximal to the heart. The BI sensors can be supplemented by the EKG sensors. A normal, healthy, heart beats at a regular rate. Irregular heart beats, known as cardiac arrhythmia, on the other hand, may characterize an unhealthy condition. Another unhealthy condition is known as congestive heart failure (“CHF”). CHF, also known as heart failure, is a condition where the heart has inadequate capacity to pump sufficient blood to meet metabolic demand. CHF may be caused by a variety of sources, including, coronary artery disease, myocardial infarction, high blood pressure, heart valve disease, cardiomyopathy, congenital heart disease, endocarditis, myocarditis, and others. Unhealthy heart conditions may be treated using a cardiac rhythm management (CRM) system. Examples of CRM systems, or pulse generator systems, include defibrillators (including implantable cardioverter defibrillator), pacemakers and other cardiac resynchronization devices.

In one implementation, BIA measurements can be made using an array of bipolar or tetrapolar electrodes that deliver a constant alternating current at 50 KHz frequency. Whole body measurements can be done using standard right-sided. The ability of any biological tissue to resist a constant electric current depends on the relative proportions of water and electrolytes it contains, and is called resistivity (in Ohms/cm 3). The measuring of bioimpedance to assess congestive heart failure employs the different bio-electric properties of blood and lung tissue to permit separate assessment of: (a) systemic venous congestion via a low frequency or direct current resistance measurement of the current path through the right ventricle, right atrium, superior vena cava, and subclavian vein, or by computing the real component of impedance at a high frequency, and (b) pulmonary congestion via a high frequency measurement of capacitive impedance of the lung. The resistance is impedance measured using direct current or alternating current (AC) which can flow through capacitors.

In one embodiment, a belt is worn by the patient with a plurality of BI probes positioned around the belt perimeter. The output of the tetrapolar probes is processed using a second-order Newton-Raphson method to estimate the left and right-lung resistivity values in the thoracic geometry. The locations of the electrodes are marked. During the measurements procedure, the belt is worn around the patient's thorax while sitting, and the reference electrode is attached to his waist. The data is collected during tidal respiration to minimize lung resistivity changes due to breathing, and lasts approximately one minute. The process is repeated periodically and the impedance trend is analyzed to detect CHF. Upon detection, the system provides vital parameters to a call center and the call center can refer to a physician for consultation or can call 911 for assistance.

In one embodiment, an array of noninvasive thoracic electrical bioimpedance monitoring probes can be used alone or in conjunction with other techniques such as impedance cardiography (ICG) for early comprehensive cardiovascular assessment and trending of acute trauma victims. This embodiment provides early, continuous cardiovascular assessment to help identify patients whose injuries were so severe that they were not likely to survive. This included severe blood and/or fluid volume deficits induced by trauma, which did not respond readily to expeditious volume resuscitation and vasopressor therapy. One exemplary system monitors cardiorespiratory variables that served as statistically significant measures of treatment outcomes: Qt, BP, pulse oximetry, and transcutaneous Po2 (Ptco2). A high Qt may not be sustainable in the presence of hypovolemia, acute anemia, pre-existing impaired cardiac function, acute myocardial injury, or coronary ischemia. Thus a fall in Ptco2 could also be interpreted as too high a metabolic demand for a patient's cardiovascular reserve. Too high a metabolic demand may compromise other critical organs. Acute lung injury from hypotension, blunt trauma, and massive fluid resuscitation can drastically reduce respiratory reserve.

One embodiment that measures thoracic impedance (a resistive or reactive impedance associated with at least a portion of a thorax of a living organism). The thoracic impedance signal is influenced by the patient's thoracic intravascular fluid tension, heart beat, and breathing (also referred to as “respiration” or “ventilation”). A “de” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject patient's thoracic fluid tension, and is therefore influenced by intravascular fluid shifts to and away from the thorax. Higher frequency components of the thoracic impedance signal are influenced by the patient's breathing (e.g., approximately between 0.05 Hz and 2.0 Hz inclusive) and heartbeat (e.g., approximately between 0.5 Hz and 10 Hz inclusive). A low intravascular fluid tension in the thorax (“thoracic hypotension”) may result from changes in posture. For example, in a person who has been in a recumbent position for some time, approximately ⅓ of the blood volume is in the thorax. When that person then sits upright, approximately ⅓ of the blood that was in the thorax migrates to the lower body. This increases thoracic impedance. Approximately 90% of this fluid shift takes place within 2 to 3 minutes after the person sits upright.

The accelerometer can be used to provide reproducible measurements. Body activity will increase cardiac output and also change the amount of blood in the systemic venous system or lungs. Measurements of congestion may be most reproducible when body activity is at a minimum and the patient is at rest. The use of an accelerometer allows one to sense both body position and body activity. Comparative measurements over time may best be taken under reproducible conditions of body position and activity. Ideally, measurements for the upright position should be compared as among themselves. Likewise measurements in the supine, prone, left lateral decubitus and right lateral decubitus should be compared as among themselves. Other variables can be used to permit reproducible measurements, i.e. variations of the cardiac cycle and variations in the respiratory cycle. The ventricles are at their most compliant during diastole. The end of the diastolic period is marked by the QRS on the electrocardiographic means (EKG) for monitoring the cardiac cycle. The second variable is respiratory variation in impedance, which is used to monitor respiratory rate and volume. As the lungs fill with air during inspiration, impedance increases, and during expiration, impedance decreases. Impedance can be measured during expiration to minimize the effect of breathing on central systemic venous volume. While respiration and CHF both cause variations in impedance, the rates and magnitudes of the impedance variation are different enough to separate out the respiratory variations which have a frequency of about 8 to 60 cycles per minute and congestion changes which take at least several minutes to hours or even days to occur. Also, the magnitude of impedance change is likely to be much greater for congestive changes than for normal respiratory variation. Thus, the system can detect congestive heart failure (CHF) in early stages and alert a patient to prevent disabling and even lethal episodes of CHF. Early treatment can avert progression of the disorder to a dangerous stage.

In an embodiment to monitor wounds such as diabetic related wounds, the conductivity of a region of the patient with a wound or is susceptible to wound formation is monitored by the system. The system determines healing wounds if the impedance and reactance of the wound region increases as the skin region becomes dry. The system detects infected, open, interrupted healing, or draining wounds through lower regional electric impedances. In yet another embodiment, the bioimpedance sensor can be used to determine body fat. In one embodiment, the BI system determines Total Body Water (TBW) which is an estimate of total hydration level, including intracellular and extracellular water; Intracellular Water (ICW) which is an estimate of the water in active tissue and as a percent of a normal range (near 60% of TBW); Extracellular Water (ECW) which is water in tissues and plasma and as a percent of a normal range (near 40% of TBW); Body Cell Mass (BCM) which is an estimate of total pounds/kg of all active cells; Extracellular Tissue (ECT)/Extracellular Mass (ECM) which is an estimate of the mass of all other non-muscle inactive tissues including ligaments, bone and ECW; Fat Free Mass (FFM)/Lean Body Mass (LBM) which is an estimate of the entire mass that is not fat. It should be available in pounds/kg and may be presented as a percent with a normal range; Fat Mass (FM) which is an estimate of pounds/kg of body fat and percentage body fat; and Phase Angle (PA) which is associated with both nutrition and physical fitness.

Additional sensors such as thermocouples or thermisters and/or heat flux sensors can also be provided to provide measured values useful in analysis. In general, skin surface temperature will change with changes in blood flow in the vicinity of the skin surface of an organism. Such changes in blood flow can occur for a number of reasons, including thermal regulation, conservation of blood volume, and hormonal changes. In one implementation, skin surface measurements of temperature or heat flux are made in conjunction with hydration monitoring so that such changes in blood flow can be detected and appropriately treated.

In one embodiment, the patch includes a sound transducer such as a microphone or a piezoelectric transducer to pick up sound produced by bones or joints during movement. If bone surfaces are rough and poorly lubricated, as in an arthritic knee, they will move unevenly against each other, producing a high-frequency, scratching sound. The high-frequency sound from joints is picked up by wide-band acoustic sensor(s) or microphone(s) on a patient's body such as the knee. As the patient flexes and extends their knee, the sensors measure the sound frequency emitted by the knee and correlate the sound to monitor osteoarthritis, for example.

In another embodiment, the patch includes a Galvanic Skin Response (GSR) sensor. In this sensor, a small current is passed through one of the electrodes into the user's body such as the fingers and the CPU calculates how long it takes for a capacitor to fill up. The length of time the capacitor takes to fill up allows us to calculate the skin resistance: a short time means low resistance while a long time means high resistance. The GSR reflects sweat gland activity and changes in the sympathetic nervous system and measurement variables. Measured from the palm or fingertips, there are changes in the relative conductance of a small electrical current between the electrodes. The activity of the sweat glands in response to sympathetic nervous stimulation (Increased sympathetic activation) results in an increase in the level of conductance. Fear, anger, startle response, orienting response and sexual feelings are all among the emotions which may produce similar GSR responses.

In yet another embodiment, measurement of lung function such as peak expiratory flow readings is done though a sensor such as Wright's peak flow meter. In another embodiment, a respiratory estimator is provided that avoids the inconvenience of having the patient breathing through the flow sensor. In the respiratory estimator embodiment, heart period data from EKG/ECG is used to extract respiratory detection features. The heart period data is transformed into time-frequency distribution by applying a time-frequency transformation such as short-term Fourier transformation (STFT). Other possible methods are, for example, complex demodulation and wavelet transformation. Next, one or more respiratory detection features may be determined by setting up amplitude modulation of time-frequency plane, among others. The respiratory recognizer first generates a math model that correlates the respiratory detection features with the actual flow readings. The math model can be adaptive based on pre-determined data and on the combination of different features to provide a single estimate of the respiration. The estimator can be based on different mathematical functions, such as a curve fitting approach with linear or polynomical equations, and other types of neural network implementations, non-linear models, fuzzy systems, time series models, and other types of multivariate models capable of transferring and combining the information from several inputs into one estimate. Once the math model has been generated, the respirator estimator provides a real-time flow estimate by receiving EKG/ECG information and applying the information to the math model to compute the respiratory rate. Next, the computation of ventilation uses information on the tidal volume. An estimate of the tidal volume may be derived by utilizing different forms of information on the basis of the heart period signal. For example, the functional organization of the respiratory system has an impact in both respiratory period and tidal volume. Therefore, given the known relationships between the respiratory period and tidal volume during and transitions to different states, the information inherent in the heart period derived respiratory frequency may be used in providing values of tidal volume. In specific, the tidal volume contains inherent dynamics which may be, after modeling, applied to capture more closely the behavioral dynamics of the tidal volume. Moreover, it appears that the heart period signal, itself, is closely associated with tidal volume and may be therefore used to increase the reliability of deriving information on tidal volume. The accuracy of the tidal volume estimation may be further enhanced by using information on the subjects vital capacity (i.e., the maximal quantity of air that can be contained in the lungs during one breath). The information on vital capacity, as based on physiological measurement or on estimates derived from body measures such as height and weight, may be helpful in estimating tidal volume, since it is likely to reduce the effects of individual differences on the estimated tidal volume. Using information on the vital capacity, the mathematical model may first give values on the percentage of lung capacity in use, which may be then transformed to liters per breath. T he optimizing of tidal volume estimation can based on, for example, least squares or other type of fit between the features and actual tidal volume. The minute ventilation may be derived by multiplying respiratory rate (breaths/min) with tidal volume (liters/breath).

In another embodiment, inductive plethysmography can be used to measure a cross-sectional area of the body by determining the self-inductance of a flexible conductor closely encircling the area to be measured. Since the inductance of a substantially planar conductive loop is well known to vary as, inter alia, the cross-sectional area of the loop, a inductance measurement may be converted into a plethysmographic area determination. Varying loop inductance may be measured by techniques known in the art, such as, e.g., by connecting the loop as the inductance in a variable frequency LC oscillator, the frequency of the oscillator then varying with the cross-sectional area of the loop inductance varies. Oscillator frequency is converted into a digital value, which is then further processed to yield the physiological parameters of interest. Specifically, a flexible conductor measuring a cross-sectional area of the body is closely looped around the area of the body so that the inductance, and the changes in inductance, being measured results from magnetic flux through the cross-sectional area being measured. The inductance thus depends directly on the cross-sectional area being measured, and not indirectly on an area which changes as a result of the factors changing the measured cross-sectional area. Various physiological parameters of medical and research interest may be extracted from repetitive measurements of the areas of various cross-sections of the body. For example, pulmonary function parameters, such as respiration volumes and rates and apneas and their types, may be determined from measurements of, at least, a chest transverse cross-sectional area and also an abdominal transverse cross-sectional area. Cardiac parameters, such central venous pressure, left and right ventricular volumes waveforms, and aortic and carotid artery pressure waveforms, may be extracted from repetitive measurements of transverse cross-sectional areas of the neck and of the chest passing through the heart. Timing measurements can be obtained from concurrent ECG measurements, and less preferably from the carotid pulse signal present in the neck. From the cardiac-related signals, indications of ischemia may be obtained independently of any ECG changes. Ventricular wall ischemia is known to result in paradoxical wall motion during ventricular contraction (the ischemic segment paradoxically “balloons” outward instead of normally contracting inward). Such paradoxical wall motion, and thus indications of cardiac ischemia, may be extracted from chest transverse cross-section area measurements. Left or right ventricular ischemia may be distinguished where paradoxical motion is seen predominantly in left or right ventricular waveforms, respectively. For another example, observations of the onset of contraction in the left and right ventricles separately may be of use in providing feedback to bi-ventricular cardiac pacing devices. For a further example, pulse oximetry determines hemoglobin saturation by measuring the changing infrared optical properties of a finger. This signal may be disambiguated and combined with pulmonary data to yield improved information concerning lung function.

In one embodiment, a cranial bioimpedance sensor is applied to detect fluids in the brain. The brain tissue can be modeled as an electrical circuit where cells with the lipid bilayer act as capacitors and the intra and extra cellular fluids act as resistors. The opposition to the flow of the electrical current through the cellular fluids is resistance. The system takes 50-kHz single-frequency bioimpedance measurements reflecting the electrical conductivity of brain tissue. The opposition to the flow of the current by the capacitance of lipid bilayer is reactance. In this embodiment, microamps of current at 50 kHz are applied to the electrode system. In one implementation, the electrode system consists of a pair of coaxial electrodes each of which has a current electrode and a voltage sensing electrode. For the measurement of cerebral bioimpedance, one pair of gel current electrodes is placed on closed eyelids and the second pair of voltage electrodes is placed in the suboccipital region projecting towards the foramen magnum. The electrical current passes through the orbital fissures and brain tissue. The drop in voltage is detected by the suboccipital electrodes and then calculated by the processor to bioimpedance values. The bioimpedance value is used to detect brain edema, which is defined as an increase in the water content of cerebral tissue which then leads to an increase in overall brain mass. Two types of brain edema are vasogenic or cytotoxic. Vasogenic edema is a result of increased capillary permeability. Cytotoxic edema reflects the increase of brain water due to an osmotic imbalance between plasma and the brain extracellular fluid. Cerebral edema in brain swelling contributes to the increase in intracranial pressure and an early detection leads to timely stroke intervention.

In another example, a cranial bioimpedance tomography system contructs brain impedance maps from surface measurements using nonlinear optimization. A nonlinear optimization technique utilizing known and stored constraint values permits reconstruction of a wide range of conductivity values in the tissue. In the nonlinear system, a Jacobian Matrix is renewed for a plurality of iterations. The Jacobian Matrix describes changes in surface voltage that result from changes in conductivity. The Jacobian Matrix stores information relating to the pattern and position of measuring electrodes, and the geometry and conductivity distributions of measurements resulting in a normal case and in an abnormal case. The nonlinear estimation determines the maximum voltage difference in the normal and abnormal cases.

In one embodiment, an electrode array sensor can include impedance, bio-potential, or electromagnetic field tomography imaging of cranial tissue. The electrode array sensor can be a geometric array of discrete electrodes having an equally-spaced geometry of multiple nodes that are capable of functioning as sense and reference electrodes. In a typical tomography application the electrodes are equally-spaced in a circular configuration. Alternatively, the electrodes can have non-equal spacing and/or can be in rectangular or other configurations in one circuit or multiple circuits. Electrodes can be configured in concentric layers too. Points of extension form multiple nodes that are capable of functioning as an electrical reference. Data from the multiple reference points can be collected to generate a spectrographic composite for monitoring over time.

The patient's brain cell generates an electromagnetic field of positive or negative polarity, typically in the millivolt range. The sensor measures the electromagnetic field by detecting the difference in potential between one or more test electrodes and a reference electrode. The bio-potential sensor uses signal conditioners or processors to condition the potential signal. In one example, the test electrode and reference electrode are coupled to a signal conditioner/processor that includes a lowpass filter to remove undesired high frequency signal components. The electromagnetic field signal is typically a slowly varying DC voltage signal. The lowpass filter removes undesired alternating current components arising from static discharge, electromagnetic interference, and other sources.

In one embodiment, the impedance sensor has an electrode structure with annular concentric circles including a central electrode, an intermediate electrode and an outer electrode, all of which are connected to the skin. One electrode is a common electrode and supplies a low frequency signal between this common electrode and another of the three electrodes. An amplifier converts the resulting current into a voltage between the common electrode and another of the three electrodes. A switch switches between a first circuit using the intermediate electrode as the common electrode and a second circuit that uses the outer electrode as a common electrode. The sensor selects depth by controlling the extension of the electric field in the vicinity of the measuring electrodes using the control electrode between the measuring electrodes. The control electrode is actively driven with the same frequency as the measuring electrodes to a signal level taken from one of the measuring electrodes but multiplied by a complex number with real and imaginary parts controlled to attain a desired depth penetration. The controlling field functions in the manner of a field effect transistor in which ionic and polarization effects act upon tissue in the manner of a semiconductor material.

With multiple groups of electrodes and a capability to measure at a plurality of depths, the system can perform tomographic imaging or measurement, and/or object recognition. In one embodiment, a fast reconstruction technique is used to reduce computation load by utilizing prior information of normal and abnormal tissue conductivity characteristics to estimate tissue condition without requiring full computation of a non-linear inverse solution.

In another embodiment, the bioimpedance system can be used with electro-encephalograph (EEG) or ERP. Since this embodiment collects signals related to blood flow in the brain, collection can be concentrated in those regions of the brain surface corresponding to blood vessels of interest. A headcap with additional electrodes placed in proximity to regions of the brain surface fed by a blood vessel of interest, such as the medial cerebral artery enables targeted information from the regions of interest to be collected. The headcap can cover the region of the brain surface that is fed by the medial cerebral artery. Other embodiments of the headcap can concentrate electrodes on other regions of the brain surface, such as the region associated with the somatosensory motor cortex. In alternative embodiments, the headcap can cover the skull more completely. Further, such a headcap can include electrodes thoughout the cap while concentrating electrodes in a region of interest. Depending upon the particular application, arrays of 1-16 head electrodes may be used, as compared to the International 10/20 system of 19-21 head electrodes generally used in an EEG instrument.

In one implementation, each amplifier for each EEG channel is a high quality analog amplifier device. Full bandwidth and ultra-low noise amplification are obtained for each electrode. Low pass, high pass, hum notch filters, gain, un-block, calibration and electrode impedance check facilities are included in each amplifier. All 8 channels in one EEG amplifier unit have the same filter, gain, etc. settings. Noise figures of less than 0.1 uVr.m.s. are achieved at the input and optical coupling stages. These figures, coupled with good isolation/common mode rejection result in signal clarity. Nine high pass filter ranges include 0.01 Hz for readiness potential measurement, and 30 Hz for EMG measurement.

In one embodiment, stimulations to elicit EEG signals are used in two different modes, i.e., auditory clicks and electric pulses to the skin. The stimuli, although concurrent, are at different prime number frequencies to permit separation of different evoked potentials (EPs) and avoid interference. Such concurrent stimulations for EP permit a more rapid, and less costly, examination and provide the patient's responses more quickly. Power spectra of spontaneous EEG, waveshapes of Averaged Evoked Potentials, and extracted measures, such as frequency specific power ratios, can be transmitted to a remote receiver. The latencies of successive EP peaks of the patient may be compared to those of a normal group by use of a normative template. To test for ischemic stroke or intracerebral or subarachnoid hemorrhage, the system provides a blood oxygen saturation monitor, using an infra-red or laser source, to alert the user if the patient's blood in the brain or some brain region is deoxygenated.

A stimulus device may optionally be placed on each subject, such as an audio generator in the form of an ear plug, which produces a series of “click” sounds. The subject's brain waves are detected and converted into audio tones. The device may have an array of LED (Light Emitting Diodes) which blink depending on the power and frequency composition of the brain wave signal. Power ratios in the frequencies of audio or somatosensory stimuli are similarly encoded. The EEG can be transmitted to a remote physician or medical aide who is properly trained to determine whether the patient's brain function is abnormal and may evaluate the functional state of various levels of the patient's nervous system.

In another embodiment, three pairs of electrodes are attached to the head of the subject under examination via tape or by wearing a cap with electrodes embedded. In one embodiment, the electrode pairs are as follows:
1) top of head to anterior throat
2) inion-nasion
3) left to right mastoid (behind ear).

A ground electrode is located at an inactive site of the upper part of the vertebral column. The electrodes are connected to differential amplification devices as disclosed below. Because the electrical charges of the brain are so small (on the order of microvolts), amplification is needed. The three amplified analog signals are converted to digital signals and averaged over a certain number of successive digital values to eliminate erroneous values originated by noise on the analog signal.

All steps defined above are linked to a timing signal which is also responsible for generating stimuli to the subject. The responses are processed in a timed relation to the stimuli and averaged as the brain responds to these stimuli. Of special interest are the responses within certain time periods and time instances after the occurrence of a stimulus of interest. These time periods and instances and their references can be:
25 to 60 milliseconds: P1-N1
180 to 250 milliseconds: N2
100 milliseconds: N100
200 milliseconds: P2
300 milliseconds: P300.

In an examination two stimuli sets may be used in a manner that the brain has to respond to the two stimuli differently, one stimulus has a high probability of occurrence, and the other stimulus is a rare occurring phenomena. The rare response is the response of importance. Three response signals are sensed and joined into a three dimensional cartesian system by a mapping program. The assignments can be
nasion-inion=X,
left-right mastoid=Y, and
top of head to anterior throat=Z.

The assignment of the probes to the axes and the simultaneous sampling of the three response signals at the same rate and time relative to the stimuli allows to real-time map the electrical signal in a three dimensional space. The signal can be displayed in a perspective representation of the three dimensional space, or the three components of the vector are displayed by projecting the vector onto the three planes X-Y, Y-Z, and X-Z, and the three planes are inspected together or separately. Spatial information is preserved for reconstruction as a map. The Vector Amplitude (VA) measure provides information about how far from the center of the head the observed event is occurring; the center of the head being the center (0,0,0) of the coordinate system.

The cranial bioimpedance sensor can be applied singly or in combination with a cranial blood flow sensor, which can be optical, ultrasound, electromagnetic sensor(s) as described in more details below. In an ultrasound imaging implementation, the carotid artery is checked for plaque build-up. Atherosclerosis is systemic—meaning that if the carotid artery has plaque buildup, other important arteries, such as coronary and leg arteries, might also be atherosclerotic.

In another embodiment, an epicardial array monopolar ECG system converts signals into the multichannel spectrum domain and identifies decision variables from the autospectra. The system detects and localizes the epicardial projections of ischemic myocardial ECGs during the cardiac activation phase. This is done by transforming ECG signals from an epicardial or torso sensor array into the multichannel spectral domain and identifying any one or more of a plurality of decision variables. The ECG array data can be used to detect, localize and quantify reversible myocardial ischemia.

In yet another embodiment, a trans-cranial Doppler velocimetrysensor provides a non-invasive technique for measuring blood flow in the brain. An ultrasound beam from a transducer is directed through one of three natural acoustical windows in the skull to produce a waveform of blood flow in the arteries using Doppler sonography. The data collected to determine the blood flow may include values such as the pulse cycle, blood flow velocity, end diastolic velocity, peak systolic velocity, mean flow velocity, total volume of cerebral blood flow, flow acceleration, the mean blood pressure in an artery, and the pulsatility index, or impedance to flow through a vessel. From this data, the condition of an artery may be derived, those conditions including stenosis, vasoconstriction, irreversible stenosis, vasodilation, compensatory vasodilation, hyperemic vasodilation, vascular failure, compliance, breakthrough, and pseudo-normalization.

In one embodiment, the system can detect numbness or weakness of the face, arm or leg, especially on one side of the body. The system detects sudden confusion, trouble speaking or understanding, sudden trouble seeing in one or both eyes, sudden trouble walking, dizziness, loss of balance or coordination, or sudden, severe headache with no known cause. In one embodiment to detect heart attack, the system detects discomfort in the center of the chest that lasts more than a few minutes, or that goes away and comes back. Symptoms can include pain or discomfort in one or both arms, the back, neck, jaw or stomach. The system can also monitor for shortness of breath which may occur with or without chest discomfort. Other signs may include breaking out in a cold sweat, nausea or lightheadedness. In order to best analyze a patient's risk of stroke, additional patient data is utilized by a stroke risk analyzer. This data may include personal data, such as date of birth, ethnic group, sex, physical activity level, and address. The data may further include clinical data such as a visit identification, height, weight, date of visit, age, blood pressure, pulse rate, respiration rate, and so forth. The data may further include data collected from blood work, such as the antinuclear antibody panel, B-vitamin deficiency, C-reactive protein value, calcium level, cholesterol levels, entidal CO2, fibromogin, amount of folic acid, glucose level, hematocrit percentage, H-pylori antibodies, hemocysteine level, hypercapnia, magnesium level, methyl maloric acid level, platelets count, potassium level, sedrate (ESR), serum osmolality, sodium level, zinc level, and so forth. The data may further include the health history data of the patient, including alcohol intake, autoimmune diseases, caffeine intake, carbohydrate intake, carotid artery disease, coronary disease, diabetes, drug abuse, fainting, glaucoma, head injury, hypertension, lupus, medications, smoking, stroke, family history of stroke, surgery history, for example. The automated analyzer can also consider related pathologies in analyzing a patient's risk of stroke, including but not limited to gastritis, increased intracranial pressure, sleep disorders, small vessel disease, and vasculitis.

An exemplary band-aid or patch with flexible circuits thereon is discussed next. The patch may be applied to a person's skin by anyone including the person themselves or an authorized person such as a family member or physician. The adhesive patch can have a gauze pad attached to one side of a backing, preferably of plastic, and wherein the pad can have an impermeable side coating with backing and a module which contains electronics for communicating with the mesh network and for sensing acceleration and bioimpedance, EKG/ECG, heart sound, microphone, optical sensor, or ultrasonic sensor in contacts with a wearer's skin. In one embodiment, the module has a skin side that may be coated with a conductive electrode lotion or gel to improve the contact. The entire patch described above may be covered with a plastic or foil strip to retain moisture and retard evaporation by a conductive electrode lotion or gel provided improve the electrode contact. In one embodiment, an acoustic sensor (microphone or piezoelectric sensor) and an electrical sensor such as EKG sensor contact the patient with a conductive gel material. The conductive gel material provides transmission characteristics so as to provide an effective acoustic impedance match to the skin in addition to providing electrical conductivity for the electrical sensor. The acoustic transducer can be directed mounted on the conductive gel material substantially with or without an intermediate air buffer. The entire patch is then packaged as sterile as are other over-the-counter adhesive bandages. When the patch is worn out, the module may be removed and a new patch backing may be used in place of the old patch. One or more patches may be applied to the patient's body and these patches may communicate wirelessly using the mesh network or alternatively they may communicate through a personal area network using the patient's body as a communication medium.

The term “positional measurement,” as that term is used herein, is not limited to longitude and latitude measurements, or to metes and bounds, but includes information in any form from which geophysical positions can be derived. These include, but are not limited to, the distance and direction from a known benchmark, measurements of the time required for certain signals to travel from a known source to the geophysical location where the signals may be electromagnetic or other forms, or measured in terms of phase, range, Doppler or other units

The system can include a contact lens with flexible circuits thereon or an eye glass with flexible circuits thereon. The contact lens can detect glucose levels using the sensors detailed above. In addition, the contact lens can be placed on eyeglasses to provide augmented reality. The contact lens or a sunglass or eyeglass embodiment contains electronics for communicating with the mesh network and for sensing acceleration and bioimpedance, EKG/ECG, EMG, heart sound, microphone, optical sensor, or ultrasonic sensor in contacts with a wearer's skin. In one embodiment, the ear module contains optical sensors to detect temperature, blood flow and blood oxygen level as well as a speaker to provide wireless communication or hearing aid. The blood flow or velocity information can be used to estimate blood pressure. The side module can contain an array of bioimpedance sensors such as bipolar or tetrapolarbioimpedance probes to sense fluids in the brain. Additional bioimpedance electrodes can be positioned around the rim of the glasses as well as the glass handle or in any spots on the eyewear that contacts the user. The side module can also contain one or more EKG electrodes to detect heart beat parameters and to detect heart problems. The side module can also contain piezoelectric transducers or microphones to detect heart activities near the brain. The side module can also contain ultrasound transmitter and receiver to create an ultrasound model of brain fluids. In one embodiment, an acoustic sensor (microphone or piezoelectric sensor) and an electrical sensor such as EKG sensor contact the patient with a conductive gel material. The conductive gel material provides transmission characteristics so as to provide an effective acoustic impedance match to the skin in addition to providing electrical conductivity for the electrical sensor. The acoustic transducer can be directed mounted on the conductive gel material substantially with or without an intermediate air buffer. In another embodiment, electronics components are distributed between first and second ear stems. In yet another embodiment, the method further comprises providing a nose bridge, wherein digital signals generated by the electronics circuit are transmitted across the nose bridge. The eyewear device may communicate wirelessly using the mesh network or alternatively they may communicate through a personal area network using the patient's body as a communication medium. Voice can be transmitted over the mesh wireless network. The speaker can play digital audio file, which can be compressed according to a compression format. The compression format may be selected from the group consisting of: PCM, DPCM, ADPCM, AAC, RAW, DM, RIFF, WAV, BWF, AIFF, AU, SND, CDA, MPEG, MPEG-1, MPEG-2, MPEG-2.5, MPEG-4, MPEG-J, MPEG 2-ACC, MP3, MP3 Pro, ACE, MACE, MACE-3, MACE-6, AC-3, ATRAC, ATRAC3, EPAC, Twin VQ, VQF, WMA, WMA with DRM, DTS, DVD Audio, SACD, TAC, SHN, OGG, OggVorbis, OggTarkin, OggTheora, ASF, LQT, QDMC, A2b, .ra, .rm, and Real Audio G2, RMX formats, Fairplay, Quicktime, SWF, and PCA, among others.

In one embodiment, the eye wear device can provide a data port, wherein the data port is carried by the ear stem. The data port may be a mini-USB connector, a FIREWIRE connector, an IEEE 1394 cable connector, an RS 232 connector, a JTAB connector, an antenna, a wireless receiver, a radio, an RF receiver, or a Bluetooth receiver. In another embodiment, the wearable device is removably connectable to a computing device. The wearable wireless audio device may be removably connectable to a computing device with a data port, wherein said data port is mounted to said wearable wireless audio device. In another embodiment, projectors can project images on the glasses to provide head-mounted display on the eye wear device. The processor can display fact, figure, to do list, and reminders need in front of the user's eyes.

One aspect addresses: WEARABLE EXOSKELETON AND FES SUPPORT

A smart device, comprising:
a flexible body wearable textile with a processor and sensors therein;
an exoskeleton with one or more joint actuators on the textile and positioned near joints; and
a functional electrical stimulus (FES) generator operating in conjunction with the exoskeleton to enable a user to ambulate.

Another aspect is DETECTING & CONTROLLING MUSCLE FIRINGS USING PATTERN RECOGNIZERS

A smart device, comprising:
a body wearable device with a processor and sensors therein;
a muscle activation recognizer coupled to the processors and sensors, the muscle activation recognizer decoding muscle neuron firings driving a predetermined muscle activity; and
a functional electrical stimulus (FES) generator coupled to the muscle activation recognizer to cause neuron firings in a disabled body portion to enable a user to control the predetermined muscle activity.

Yet another aspect is for EYE BASED FES CONTROL

1. A monitoring system for a person, comprising:

a projector aimed at a retina, the projector providing 3D images with different depth view points, the projector having a camera to capture one or more obstacles;

a processor coupled to the camera and coupled to an in-door navigation system; and

a functional electrical stimulus (FES) generator controlled by the processor to help the person to avoid the obstacle.

2. The system of claim 1, comprising a gyroscope coupled to the projector.

3. The system of claim 1, comprising a sound transducer coupled to the wireless transceiver to communicate audio.

4. The system of claim 1, comprising one of: EEG detector, EKG detector, ECG detector, electromagnetic detector, ultrasonic detector, optical detector.

5. The system of claim 1, comprising an accelerometer to monitor patient movement.

6. The system of claim 1, wherein the electrodes are mounted on a back of a skin-contacting case.

7. The system of claim 1, comprising a call center to provide a human response.

8. The system of claim 1, comprising a web server coupled to the wireless network and to the POTS to provide information to an authorized remote user.

9. The system of claim 1, wherein the sensor monitors blood pressure.

10. The system of claim 1, wherein the sensor monitors EEG to identify a seizure.

11. The system of claim 1, comprising code to display heart waveforms on a mobile device.

12. The system of claim 1, wherein the sensor determines one of: total body water, compartmentalization of body fluids, cardiac monitoring, blood flow, skinfold thickness, dehydration, blood loss, wound monitoring, ulcer detection, deep vein thrombosis, hypovolemia, hemorrhage, blood loss.

13. The system of claim 1, comprising a patch having a bioelectric impedance (BI) sensor in communication with the wireless transceiver.

Yet another aspect is MUSCLE BASED MEDICAL MONITORING

1. A monitoring system for a person, comprising:

an EMG sensor coupled to the person;

a pattern recognizer receiving EMG data from the EMG sensor, the pattern recognizer detecting deviations from normal EMG data and sending a medical alert.

2. The system of claim 1, comprising a camera mounted on the lens and aimed at the retina.

3. The system of claim 1, comprising a sound transducer coupled to the wireless transceiver to communicate audio.

4. The system of claim 1, comprising one of: EEG detector, EKG detector, ECG detector, electromagnetic detector, ultrasonic detector, optical detector.

5. The system of claim 1, comprising an accelerometer to monitor patient movement.

6. The system of claim 1, wherein the electrodes are mounted on a back of a skin-contacting case.

7. The system of claim 1, comprising a call center to provide a human response.

8. The system of claim 1, comprising a web server coupled to the wireless network and to the POTS to provide information to an authorized remote user.

9. The system of claim 1, wherein the sensor monitors blood pressure.

10. The system of claim 1, wherein the sensor monitors EEG to identify a seizure.

11. The system of claim 1, comprising code to display heart waveforms on a mobile device.

12. The system of claim 1, wherein the sensor determines one of: total body water, compartmentalization of body fluids, cardiac monitoring, blood flow, skinfold thickness, dehydration, blood loss, wound monitoring, ulcer detection, deep vein thrombosis, hypovolemia, hemorrhage, blood loss.

13. The system of claim 1, comprising a patch having a bioelectric impedance (BI) sensor in communication with the wireless transceiver.

14. The system of claim 1, wherein the transceiver transmits and receives voice from the person over a wireless network to one of: a doctor, a nurse, a medical assistant, a caregiver, an emergency response unit, a family member.

15. The system of claim 1, comprising code to store and analyze patient information.

16. The system of claim 15, wherein the patient information includes medicine taking habits, eating and drinking habits, sleeping habits, or exercise habits.

17. The system of claim 1, wherein the sensor monitors glucose level.

18. A monitoring system, comprising:

a projector aimed at a retina, the projector providing 3D images with different depth view points;

a mobile device;

a sensor including one or more electrodes mounted on a case to contact a patient, the sensor having a wireless transceiver adapted to communicate with the mobile device; and

a software module to display vital signs on the mobile device.

19. The system of claim 18, comprising an EMG sensor.

20. The system of claim 18, comprising code for sensing eye health, code for emotion sensing, or code for authenticating access to a secured device based on retinal blood vessel dilation.

Yet another aspect is INCONTINENCE MONITORING AND CONTROL

1. A monitoring system for a person, comprising:

an EMG sensor coupled to the person;

a pattern recognizer receiving EMG data from the EMG sensor, the pattern recognizer detecting an incontinence condition and actuating a functional electrical stimulation (FES) device to control the body in response thereto.

2. The system of claim 1, comprising a camera mounted on the lens and aimed at the retina.

3. The system of claim 1, comprising a sound transducer coupled to the wireless transceiver to communicate audio.

4. The system of claim 1, comprising one of: EEG detector, EKG detector, ECG detector, electromagnetic detector, ultrasonic detector, optical detector.

5. The system of claim 1, comprising an accelerometer to monitor patient movement.

6. The system of claim 1, wherein the electrodes are mounted on a back of a skin-contacting case.

7. The system of claim 1, comprising a call center to provide a human response.

8. The system of claim 1, comprising a web server coupled to the wireless network and to the POTS to provide information to an authorized remote user.

9. The system of claim 1, wherein the sensor monitors blood pressure.

10. The system of claim 1, wherein the sensor monitors EEG to identify a seizure.

11. The system of claim 1, comprising code to display heart waveforms on a mobile device.

12. The system of claim 1, wherein the sensor determines one of: total body water, compartmentalization of body fluids, cardiac monitoring, blood flow, skinfold thickness, dehydration, blood loss, wound monitoring, ulcer detection, deep vein thrombosis, hypovolemia, hemorrhage, blood loss.

13. The system of claim 1, comprising a patch having a bioelectric impedance (BI) sensor in communication with the wireless transceiver.

14. The system of claim 1, wherein the transceiver transmits and receives voice from the person over a wireless network to one of: a doctor, a nurse, a medical assistant, a caregiver, an emergency response unit, a family member.

15. The system of claim 1, comprising code to store and analyze patient information.

16. The system of claim 15, wherein the patient information includes medicine taking habits, eating and drinking habits, sleeping habits, or exercise habits.

17. The system of claim 1, wherein the sensor monitors glucose level.

18. A monitoring system, comprising:

a projector aimed at a retina, the projector providing 3D images with different depth view points;

a mobile device;

a sensor including one or more electrodes mounted on a case to contact a patient, the sensor having a wireless transceiver adapted to communicate with the mobile device; and

a software module to display vital signs on the mobile device.

19. The system of claim 18, comprising an EMG sensor.

20. The system of claim 18, comprising code for sensing eye health, code for emotion sensing, or code for authenticating access to a secured device based on retinal blood vessel dilation.

Another aspect is PAIN MANAGEMENT
1. A system to treat a person, comprising:

a sensor detecting a pain experienced by the person at a selected time; and

a functional electrical stimulation (FES) device to distract the person at the selected time.
2. The system of claim 1, wherein the head-mounted device comprises an augmented reality device or a virtual reality device.
3. The system of claim 1, wherein the content generator comprises a game or a video.
4. The system of claim 1, wherein the content generator displays content to cause a rush in the person.
5. The system of claim 1, wherein the content generator displays content to cause biological generation of adrenaline in the person.
6. The system of claim 1, comprising sensors to detect pain or discomfort.
7. The system of claim 6, wherein one of the sensors comprises biofeedback sensor, electromyogram (EMG) sensors, electroencephalography (EEG), electrophysiological sensor, electrocorticography (ECoG) sensor, magnetoencephalography (MEG) sensor, positron emission tomography (PET) sensor, functional magnetic resonance imaging (fMRI) sensor, optical imaging sensor, functional Near InfraRed (fNIR) sensor.
8. The system of claim 1, wherein the person is distract at a pain portion of an operation, a treatment, a biological sampling, an irradiation process, or a body scan.
9. The system of claim 1, comprising:

positioning the patient in a targeted area for a medical mission;

sensing biometric and physical conditions of a patient during the mission, and

keeping the patient in a predetermined position with a game or video during medical mission.
10. The system of claim 1, comprising sharing images of a procedure from a healthcare provider with the patient.
11. The system of claim 1, wherein the sensing comprises tracking motion or capturing biofeedback data.
12. The system of claim 1, wherein the sensor determines one of: total body water, compartmentalization of body fluids, cardiac monitoring, blood flow, skinfold thickness, dehydration, blood loss, wound monitoring, ulcer detection, deep vein thrombosis, hypovolemia, hemorrhage, blood loss.
13. The system of claim 1, comprising code to perform gesture recognition, facial recognition and voice recognition.
14. The system of claim 1, comprising a 3 -D body sensor, wherein the treatment processor compares a patient position to a reference position, and providing feedback to a patient to move to the reference position.
15. The system of claim 1, comprising a game that mentally shocks the person at the selected time.
16. The system of claim 1, wherein the game provides a virtual world with an avatar for the person, wherein the avatar moves based on the person's head movement.
17. A system for monitoring a patient, comprising:

sensors to detect pain based on biometric and physical conditions of a patient; and

a functional electrical stimulation (FES) device blocking pain when the sensors detect a pain condition for the patient.
18. The system of claim 17, comprising a projector aimed at a retina, the projector providing 3D images with different depth view points.
19. A monitoring system, comprising:

a projector aimed at a retina, the projector providing 3D images with different depth view points;

a camera to capture vision and transmitting the vision to the projector to paint the retina with images associated with the vision;

a mobile device coupled to the projector;

sensors coupled to a body to detect pain based on biometric and physical conditions, the sensor having a wireless transceiver adapted to communicate with the mobile device; and

a functional electrical stimulation (FES) device to provide stimulation during pain episodes to distract the patient from feeling the pain.
20. The system of claim 19, wherein the FES drives biological generation of adrenaline in a person.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A method for assisting a user, the method comprising:
during a training phase, electrically capturing muscle signals associated with a predetermined task from one or more people and training a learning machine including a neural network, a statistical recognizer, or a hidden markov model to associate the electrically captured muscle signals with the predetermined task, wherein the predetermined task includes one of: daily movement, living pattern, walking, locomotion, hand movement, finger movement, and gesture;
during use of the learning machine to assist the user, identifying a desired task to the learning machine to generate signals associated with the muscle movement; and
applying functional electrical stimulation (FES) to the user to actuate the muscle signals for the desired task.

2. The method of claim 1, comprising learning sub-muscle movement grammars for the desired task.

3. The method of claim 1, wherein the muscle signals comprise a plurality of sub-muscle signals to granularly form a movement.

4. The method of claim 1, wherein the learning machine learns ambulatory muscle control.

5. The method of claim 1, wherein the learning machine learns arm or hand control.

6. The method of claim 1, wherein the learning machine learns muscle signals for walking, sitting, standing, or controlling a vehicle.

7. The method of claim 1, wherein the learning machine learns ambulatory muscle control.

8. The method of claim 1, wherein the learning machine learns control of one or more of the following muscles: Trapezius, Levator Scapulae, Major Rhomboids, Minor Rhomboids, Supraspinatus, Infraspinatus, Teres Minor, pronator teres, Gluteus Maximus, Sternocleidomastoid, rectus abdominus, and deltoid.

9. The method of claim 1, wherein the learning machine learns sacral nerve stimulation to reduce weight.

10. The method of claim 1, wherein the learning machine learns heart nerve stimulation to control blood pressure or to reduce risk of heart failure or heart attack.

11. The method of claim 1, comprising capturing electrical signals near a sacral nerve, wherein the learning machine learns sacral nerve stimulation to control bowel movement, bladder movement, or incontinence.

12. The method of claim 1, comprising retrieving information from servers associated with at least one or more social networking platforms.

13. The method of claim 1, comprising rendering virtual content includes rendering at least a portion of the virtual content including background scenery depicting a type of activity the user is interested in performing and one or more participants with whom the user is willing to participate in the activity.

14. The method of claim 13, wherein the type of activity that a user is interested in performing and the participants with whom the user is willing to participate in the activity are determined from one or more among previous activities performed by the user and a set of predefined criteria, which includes preference and interest.

15. The method of claim 1, wherein virtual content is rendered based on the user's selection of participants and activity.

16. The method of claim 1, wherein the displayed virtual content is possible to be altered by the user by providing input corresponding to of activity types and the participants.

17. The method of claim 1, wherein a displayed virtual content is altered if the displayed virtual content does not match the activity or participants.

18. The method of claim 1, wherein the user is provided an option to select participants to perform the activity comprising broadcasting requests to one or more other users to participate.

19. The method of claim 1, comprising remotely receiving signals from at least another user to provide to the FES and allowing a remote unit to control muscles to perform the desired task.

20. A method for enabling a user to participate in an activity with one or more other users, the method comprising:
capturing electrical signals associated with muscle activities in performing a task and training a neural network to associate one or more muscle signals with a task from a set of operations including one of: daily movement, living pattern, walking, locomotion, hand movement, finger movement, gesture; and
in response to a physical or a virtual task, applying the learning machine to apply functional electrical stimulation (FES) to apply electrical signals to move one or more muscles responsive to the task.
